(19) United States
(12) Patent Application Publication
(10) Pub. No.: US 2025/0082657 A1
(43) Pub. Date: Mar. 13, 2025

(54) CONJUGATES OF CHEMOTHERAPY AGENTS AND TISSUE-BINDING SMALL MOLECULES, COMPOSITIONS AND METHODS THEREOF

(71) Applicant: CANWELL BIOTECH LIMITED, Hong Kong (CN)

(72) Inventors: NINGHUI YU, Wellesley, MA (US); RONGLIANG LOU, Cheshire, CT (US)

(21) Appl. No.: 18/727,076

(22) PCT Filed: Feb. 3, 2023
(86) PCT No.: PCT/CN2023/074329

§ 371 Date: Jul. 6, 2024

Related U.S. Application Data
(63) Continuation-in-part of application No. PCT/CN2022/075427, filed on Feb. 8, 2022.

(30) Foreign Application Priority Data
Feb. 8, 2022 (WO) PCT/CN2022/075427

(51) Int. Cl. A61K 31/69; A61K 31/4745; A61P 35/00; C07D 491/22; C07F 5/02; C07F 13/00

(52) U.S. Cl. CPC A61K 31/69; A61K 31/4745; A61P 35/00; C07D 491/22; C07F 5/025; C07F 13/00

(57) ABSTRACT

The invention provides novel conjugates of tissue-binding small molecules and therapeutic agents and pharmaceutical compositions thereof, and their use in and methods of treatment of certain diseases or conditions (e.g., cancer).

### TECHNICAL FIELD OF THE INVENTION

The invention generally relates to novel compounds and therapeutic uses thereof. More particularly, the invention provides conjugates of tissue-binding small molecules and therapeutic agents (e.g., anticancer agents) and pharmaceutical compositions thereof, and their use in and methods of treatment of certain diseases or conditions (e.g., cancer).

### BACKGROUND OF THE INVENTION

Immunotherapy approaches treatment of diseases by activating or suppressing the patient's immune system. It has gained great interest from researchers and clinicians over the past decade, particularly due to its promise to treat various forms of cancer. (Syn, et al. 2017 The Lancet Oncol. 18 (12): e731-e741; Conforti L 2012 Clin. Immunol. 142 (2): 105-106; Nishino, et al. 2017 Nat. Rev. Clin. Oncol. 14 (11): 655-668).) To improve treatment outcomes, immunotherapeutic treatments need to be combined with small molecule drugs such as chemotherapy drugs, kinase inhibitors, indoleamine-2,3-dioxygenase 1 (IDO-1) and adenosine receptor inhibitors (A2a) inhibitors, chemokine receptor antagonists, toll-like receptors (TLRs) and stimulator of interferon genes (STING) modulators. (Huck, et al. 2018 Angew Chem. Int. Ed. 57, 4412-4428.)

Small molecule chemotherapeutic drugs remain an important part of the conventional cancer treatment and may be combined with surgery, radiotherapy and immunotherapy to improve clinical outcomes. The challenge for traditional chemotherapy is maintaining potency while reducing or avoiding side effects and toxicity resulting from systemic exposure. In addition, some drugs must be dosed frequently by intravenous (IV) injection or infusion for hours. Patient compliance and associated hospital costs can be challenging.

Thus, there remains an ongoing need for novel cancer therapies, in particular that offers good efficacy and safety profiles.

### SUMMARY OF THE INVENTION

The invention is based in part on the unexpected discovery of novel small molecule compounds, methods of their synthesis, and pharmaceutical compositions as well as methods thereof for treating or reducing various diseases or conditions.

In one aspect, the invention generally relates to a compound of any of the following formulas:

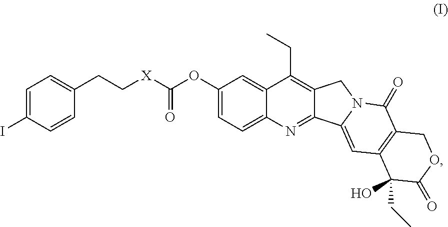

wherein X is CHR or NR, and R is H or alkyl;

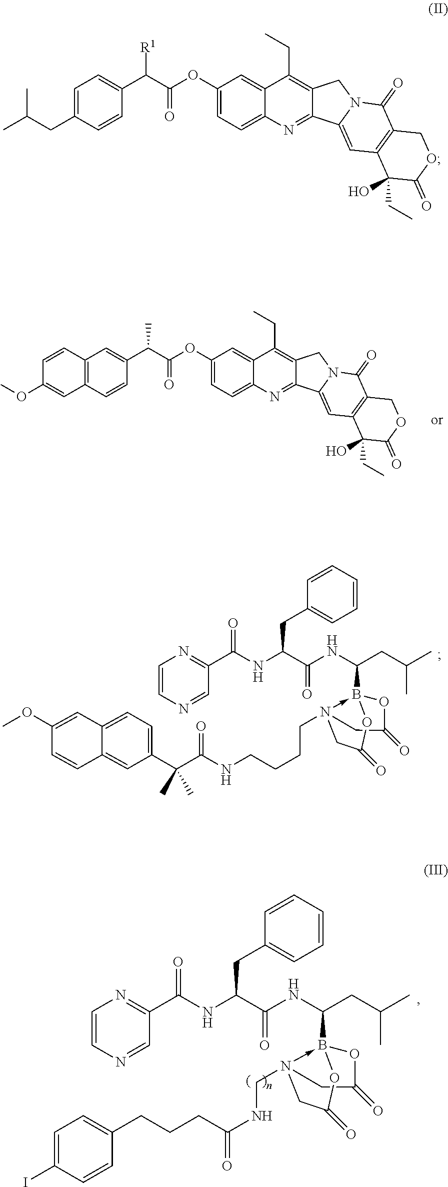

wherein n is an intergern selected from 2-8 (i.e., 2, 3, 4, 5, 6, 7 or 8),

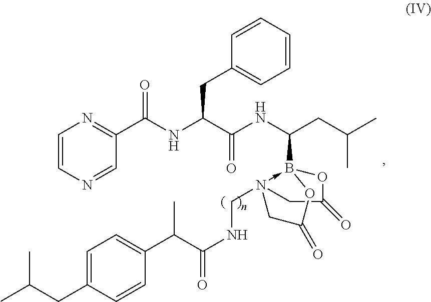

wherein n is an intergern selected from 2-10 (e.g., 2, 3, 4, 5, 6, 7, 8, 9 or 10),

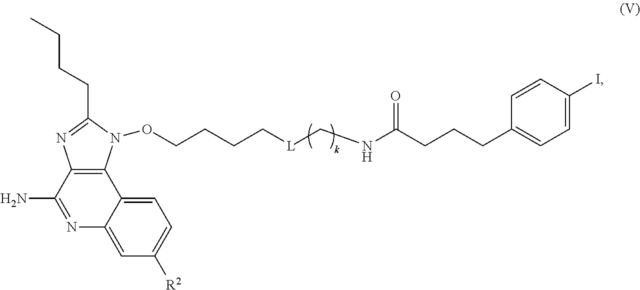

wherein R² is H or P(═O)(R³)₂ and R³ is alkyl, and L is a single bond or a group selected from:

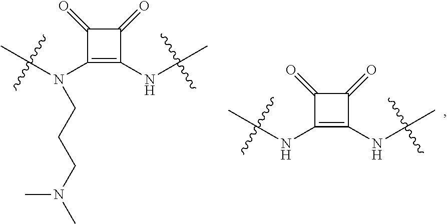 [SMILES: *Nc1c(N(*)CCCN(C)C)c(=O)c1=O ; *Nc1c(N*)c(=O)c1=O]

k is an integer selected from 0-4 (e.g., 0, 1, 2, 3 or 4);

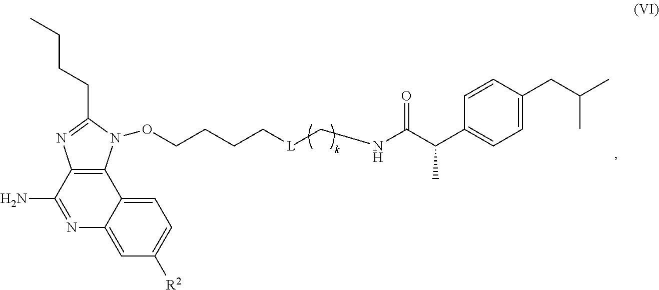

wherein R² is H or (═O)(R³)₂ and R³ is alkyl, and L is a single bond or a group selected from:

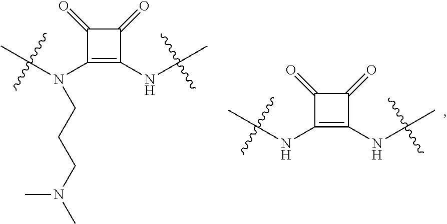 [SMILES: *Nc1c(N(*)CCCN(C)C)c(=O)c1=O ; *Nc1c(N*)c(=O)c1=O]

and k is an integer selected from 0-4 (e.g., 0, 1, 2, 3 or 4);

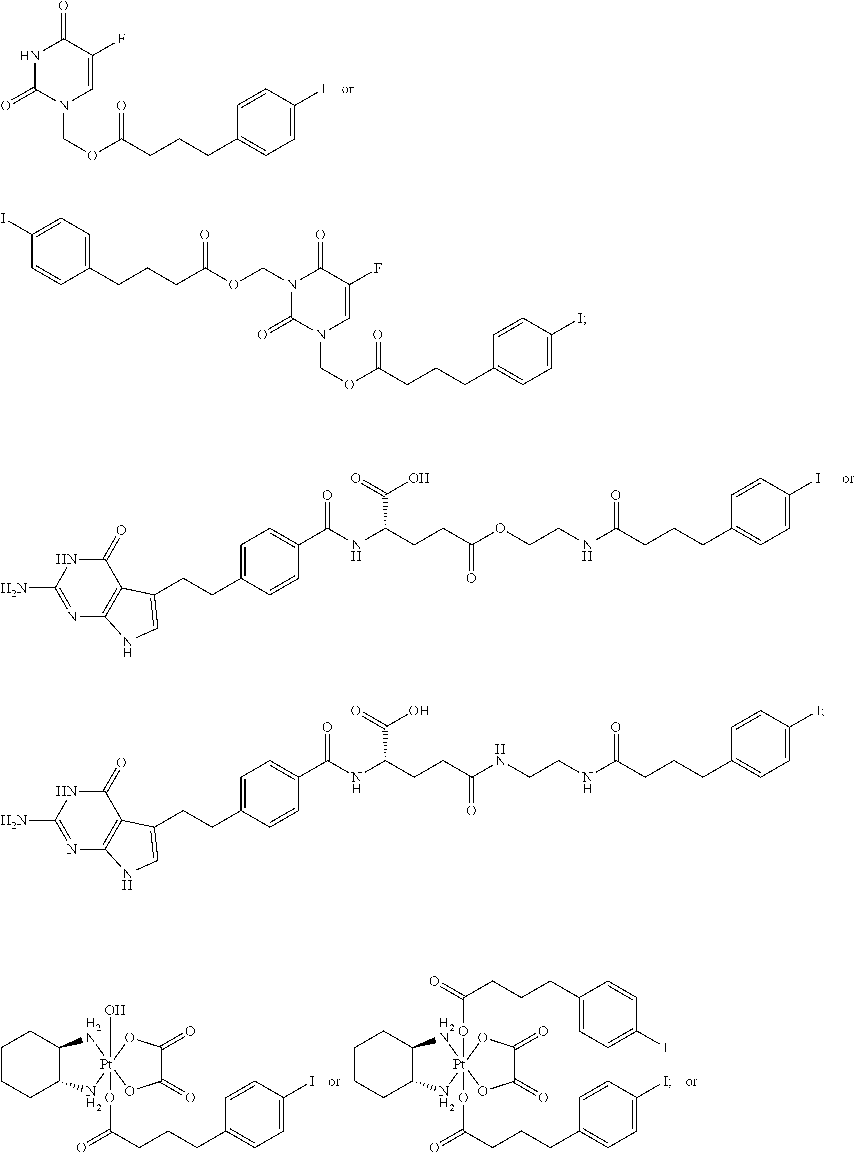

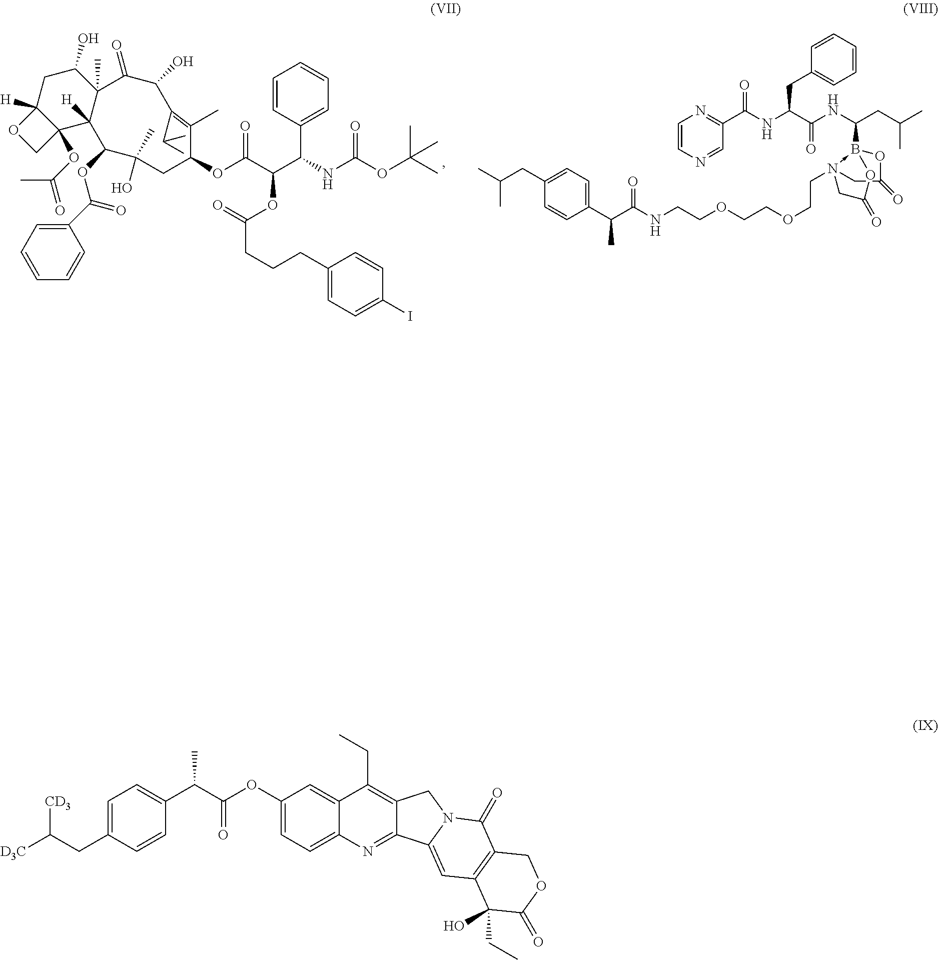

or a pharmaceutically acceptable form or an isotope derivative thereof.

In yet another aspect, the invention generally relates to a pharmaceutical composition comprising a compound disclosed herein and a pharmaceutically acceptable excipient, carrier, or diluent.

In yet another aspect, the invention generally relates to a unit dosage form comprising a pharmaceutical composition comprising a compound disclosed herein.

In yet another aspect, the invention generally relates to a method for treating or reducing a disease or condition, comprising administering to a subject in need thereof a pharmaceutical composition comprising a compound disclosed herein and a pharmaceutically acceptable excipient, carrier, or diluent.

In yet another aspect, the invention generally relates to use of a compound disclosed herein for treating or reducing a disease or condition, for example, cancer.

In yet another aspect, the invention generally relates to use of a compound disclosed herein, and a pharmaceutically acceptable excipient, carrier, or diluent, in preparation of a medicament for treating or reducing a disease or condition, for example, cancer.

### Definitions

Unless defined otherwise, all technical and scientific terms used herein have the same meaning as commonly understood by one of ordinary skill in the art to which this invention belongs. General principles of organic chemistry, as well as specific functional moieties and reactivity, are described in “Organic Chemistry”, Thomas Sorrell, University Science Books, Sausalito: 1999, and “March's Advanced Organic Chemistry”, 5^{th} Ed.: Smith, M. B. and March, J., John Wiley & Sons, New York: 2001, the entire contents of which are hereby incorporated by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic methods well known in the art, and subsequent recovery of the pure enantiomers.

Solvates and polymorphs of the compounds of the invention are also contemplated herein. Solvates of the compounds of the present invention include, for example, hydrates.

As used herein, the term “inhibit” refers to any measurable reduction of biological activity. Thus, as used herein, “inhibit” or “inhibition” may be referred to as a percentage of a normal level of activity.

As used herein, the term “effective amount” of an active agent refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the patient.

As used herein, the terms “treatment” or “treating” a disease or disorder refers to a method of reducing, delaying or ameliorating such a condition before or after it has occurred. Treatment may be directed at one or more effects or symptoms of a disease and/or the underlying pathology. The treatment can be any reduction and can be, but is not limited to, the complete ablation of the disease or the symptoms of the disease. As compared with an equivalent untreated control, such reduction or degree of prevention is at least 5%, 10%, 20%, 40%, 50%, 60%, 80%, 90%, 95%, or 100% as measured by any standard technique.

As used herein, a “pharmaceutically acceptable form” of a disclosed compound includes, but is not limited to, pharmaceutically acceptable salts, esters, hydrates, solvates, polymorphs, isomers, prodrugs, and isotopically labeled derivatives thereof. In one embodiment, a “pharmaceutically acceptable form” includes, but is not limited to, pharmaceutically acceptable salts, esters, prodrugs and isotopically labeled derivatives thereof. In some embodiments, a “pharmaceutically acceptable form” includes, but is not limited to, pharmaceutically acceptable isomers and stereoisomers, prodrugs and isotopically labeled derivatives thereof.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchioric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The salts can be prepared in situ during the isolation and purification of the disclosed compounds, or separately, such as by reacting the free base or free acid of a parent compound with a suitable base or acid, respectively. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Organic bases from which salts can be derived include, for example, primary, secondary, and tertiary amines, substituted amines, including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt can be chosen from ammonium, potassium, sodium, calcium, and magnesium salts.

In certain embodiments, the pharmaceutically acceptable form is a “solvate” (e.g., a hydrate). As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate”. Pharmaceutically acceptable solvates and hydrates are complexes that, for example, can include 1 to about 100, or 1 to about 10, or 1 to about 2, about 3 or about 4, solvent or water molecules. It will be understood that the term “compound” as used herein encompasses the compound and solvates of the compound, as well as mixtures thereof.

In certain embodiments, the pharmaceutically acceptable form is a prodrug. As used herein, the term “prodrug” (or “pro-drug”) refers to compounds that are transformed in vivo to yield a disclosed compound or a pharmaceutically acceptable form of the compound. A prodrug can be inactive when administered to a subject, but is converted in vivo to an active compound, for example, by hydrolysis (e.g., hydrolysis in blood). In certain cases, a prodrug has improved physical and/or delivery properties over the parent compound. Prodrugs can increase the bioavailability of the compound when administered to a subject (e.g., by permitting enhanced absorption into the blood following oral administration) or which enhance delivery to a biological compartment of interest (e.g., the brain or lymphatic system) relative to the parent compound. Exemplary prodrugs include derivatives of a disclosed compound with enhanced aqueous solubility or active transport through the gut membrane, relative to the parent compound.

The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. Exemplary advantages of a prodrug can include, but are not limited to, its physical properties, such as enhanced water solubility for parenteral administration at physiological pH compared to the parent compound, or it can enhance absorption from the digestive tract, or it can enhance drug stability for long-term storage.

Prodrugs commonly known in the art include well-known acid derivatives, such as, for example, esters prepared by reaction of the parent acids with a suitable alcohol, amides prepared by reaction of the parent acid compound with an amine, basic groups reacted to form an acylated base derivative, etc. Of course, other prodrug derivatives may be combined with other features disclosed herein to enhance bioavailability. As such, those of skill in the art will appreciate that certain of the presently disclosed compounds having free amino, amido, hydroxy or carboxylic groups can be converted into prodrugs. Prodrugs include compounds having an amino acid residue, or a polypeptide chain of two or more (e.g., two, three or four) amino acid residues which are covalently joined through peptide bonds to free amino, hydroxy or carboxylic acid groups of the presently disclosed compounds. The amino acid residues include the 20 naturally occurring amino acids commonly designated by three letter symbols and also include 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Prodrugs also include compounds having a carbonate, carbamate, amide or alkyl ester moiety covalently bonded to any of the above substituents disclosed herein.

As used herein, the term “pharmaceutically acceptable” excipient, carrier, or diluent refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, the terms “isolated” or “purified” refer to a material that is substantially or essentially free from components that normally accompany it in its native state. Purity and homogeneity are typically determined using analytical chemistry techniques such as polyacrylamide gel electrophoresis or high-performance liquid chromatography.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

As used herein, the term “low dosage” refers to at least 5% less (e.g., at least 10%, 20%, 50%, 80%, 90%, or even 95%) than the lowest standard recommended dosage of a particular compound formulated for a given route of administration for treatment of any human disease or condition. For example, a low dosage of an agent that is formulated for administration by inhalation will differ from a low dosage of the same agent formulated for oral administration.

As used herein, the term “high dosage” is meant at least 5% (e.g., at least 10%, 20%, 50%, 100%, 200%, or even 300%) more than the highest standard recommended dosage of a particular compound for treatment of any human disease or condition.

Isotopically-labeled compounds are also within the scope of the present disclosure. As used herein, an “isotopically-labeled compound” or “isotope derivative” refers to a presently disclosed compound including pharmaceutical salts and prodrugs thereof, each as described herein, in which one or more atoms are replaced by an atom having an atomic mass or mass number different from the atomic mass or mass number usually found in nature. Examples of isotopes that can be incorporated into compounds presently disclosed include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O ³¹P, ³²P, ³⁵S, ¹⁸F, and ³⁶Cl, respectively.

By isotopically-labeling the presently disclosed compounds, the compounds may be useful in drug and/or substrate tissue distribution assays. Tritiated (³H) and carbon-14 (¹⁴C) labeled compounds are particularly preferred for their ease of preparation and detectability. Further, substitution with heavier isotopes such as deuterium (²H) can afford certain therapeutic advantages resulting from greater metabolic stability, for example increased in vivo half-life or reduced dosage requirements and, hence, may be preferred in some circumstances. Isotopically labeled compounds presently disclosed, including pharmaceutical salts, esters, and prodrugs thereof, can be prepared by any means known in the art. Benefits may also be obtained from replacement of normally abundant ¹²C with ¹³C. (See, WO 2007/005643, WO 2007/005644, WO 2007/016361, and WO 2007/016431.)

For example, deuterium (2H) can be incorporated into a compound disclosed herein for the purpose in order to manipulate the oxidative metabolism of the compound by way of the primary kinetic isotope effect. The primary kinetic isotope effect is a change of the rate for a chemical reaction that results from exchange of isotopic nuclei, which in turn is caused by the change in ground state energies necessary for covalent bond formation after this isotopic exchange. Exchange of a heavier isotope usually results in a lowering of the ground state energy for a chemical bond and thus causes a reduction in the rate in rate-limiting bond breakage. If the bond breakage occurs in or in the vicinity of a saddle-point region along the coordinate of a multi-product reaction, the product distribution ratios can be altered substantially. For explanation: if deuterium is bonded to a carbon atom at a non-exchangeable position, rate differences of k_{M}/k_{D}=2-7 are typical. If this rate difference is successfully applied to a compound disclosed herein that is susceptible to oxidation, the profile of this compound in vivo can be drastically modified and result in improved pharmacokinetic properties.

When discovering and developing therapeutic agents, the person skilled in the art is able to optimize pharmacokinetic parameters while retaining desirable in vitro properties. It is reasonable to assume that many compounds with poor pharmacokinetic profiles are susceptible to oxidative metabolism. In vitro liver microsomal assays currently available provide valuable information on the course of oxidative metabolism of this type, which in turn permits the rational design of deuterated compounds of those disclosed herein with improved stability through resistance to such oxidative metabolism. Significant improvements in the pharmacokinetic profiles of compounds disclosed herein are thereby obtained, and can be expressed quantitatively in terms of increases in the in vivo half-life (t/2), concen-tra-tion at maximum therapeutic effect (Cₘₐₓ), area under the dose response curve (AUC), and F; and in terms of reduced clearance, dose and materials costs.

The following is intended to illustrate the above: a compound which has multiple potential sites of attack for oxidative metabolism, for example benzylic hydrogen atoms and hydrogen atoms bonded to a nitrogen atom, is prepared as a series of analogues in which various combinations of hydrogen atoms are replaced by deuterium atoms, so that some, most or all of these hydrogen atoms have been replaced by deuterium atoms. Half-life determinations enable favorable and accurate determination of the extent of the extent to which the improvement in resistance to oxidative metabolism has improved. In this way, it is determined that the half-life of the parent compound can be extended by up to 100% as the result of deuterium-hydrogen exchange of this type.

Deuterium-hydrogen exchange in a compound disclosed herein can also be used to achieve a favorable modification of the metabolite spectrum of the starting compound in order to diminish or eliminate undesired toxic metabolites. For example, if a toxic metabolite arises through oxidative carbon-hydrogen (C—H) bond cleavage, it can reasonably be assumed that the deuterated analogue will greatly diminish or eliminate production of the unwanted metabolite, even if the particular oxidation is not a rate-determining step. Further information on the state of the art with respect to deuterium-hydrogen exchange may be found, for example in Hanzlik et al., J. Org. Chem. 55, 3992-3997, 1990, Reider et al., J. Org. Chem. 52, 3326-3334, 1987, Foster, Adv. Drug Res. 14, 1-40, 1985, Gillette et al, Biochemistry 33(10) 2927-2937, 1994, and Jarman et al. Carcinogenesis 16(4), 683-688, 1993.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

### DETAILED DESCRIPTION OF THE INVENTION

The invention provides novel small molecule compounds, methods of their synthesis, and pharmaceutical compositions as well as methods thereof for treating or reducing various diseases or conditions.

A central feature of the present invention is that compounds of the invention are cancer-targeting and slow-releasing therapeutic agents, affording targeted and sustained delivery. The conjugates of the invention are comprised of a tissue protein binder, a cleavable linker and a small molecule drug. After local injection of the conjugate, the tissue protein binder, acting as a “molecular glue”, binds to tissue proteins in solid tumor, thereby retaining the conjugates in the solid tumor without leaking to systemic circulation. The small molecule drug is then slowly released from the conjugate by breakage of the cleavable linker. Slow but sustained release of the drug inside the solid tumor amplifies the tumor-killing effect while minimizing the adverse reaction because minimum amount of the drug is leaked into systemic circulation. The dosing schedule can be varied depending on the half-life of the conjugates.

In one aspect, the invention generally relates to a compound having the structural formula of (I):

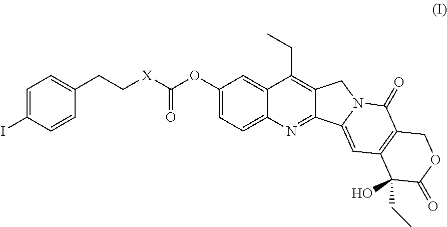

wherein
X is CHR or NR, and
R is H or alkyl,
   or a pharmaceutically acceptable form or an isotope derivative thereof.

In certain embodiments, the compound of formula (I) is selected from:

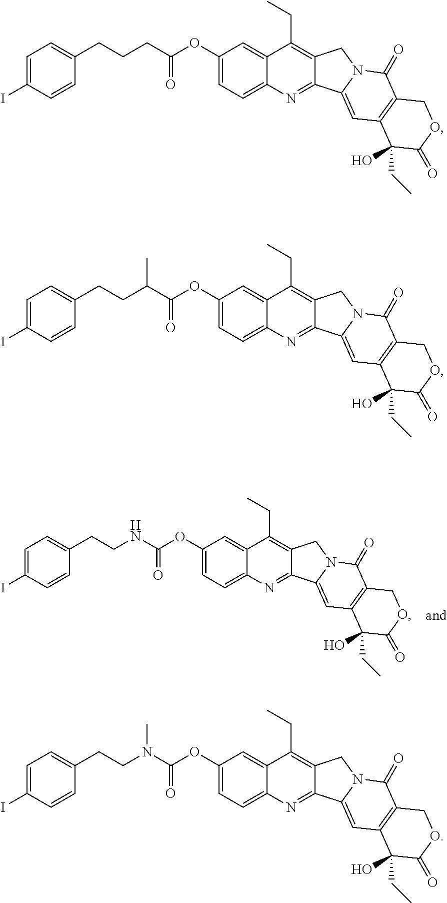 [SMILES: CCc1c2c(nc3ccc(OC(=O)CCCc4ccc(I)cc4)cc13)-c1cc3c(c(=O)n1C2)COC(=O)[C@]3(O)CC ; CCc1c2c(nc3ccc(OC(=O)C(C)CCc4ccc(I)cc4)cc13)-c1cc3c(c(=O)n1C2)COC(=O)[C@]3(O)CC ; CCc1c2c(nc3ccc(OC(=O)NCCc4ccc(I)cc4)cc13)-c1cc3c(c(=O)n1C2)COC(=O)[C@]3(O)CC ; CCc1c2c(nc3ccc(OC(=O)N(C)CCc4ccc(I)cc4)cc13)-c1cc3c(c(=O)n1C2)COC(=O)[C@]3(O)CC]

In another aspect, the invention generally relates to a compound having the structural formula of (II):

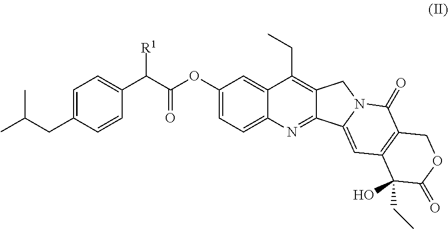

or a pharmaceutically acceptable form or an isotope derivative thereof.

In certain embodiments, the compound of formula (II) is selected from:

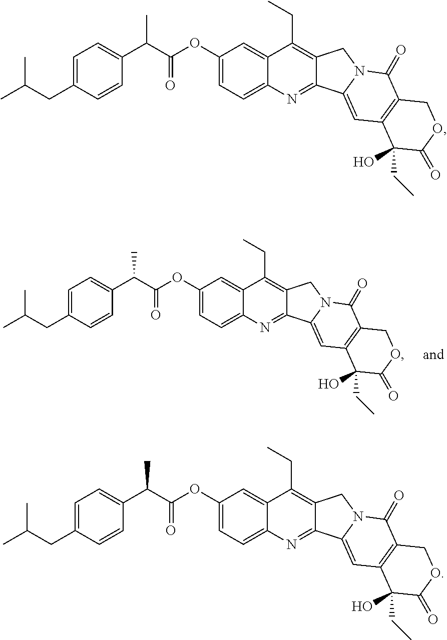 [SMILES: CCc1c2c(nc3ccc(OC(=O)C(C)c4ccc(CC(C)C)cc4)cc13)-c1cc3c(c(=O)n1C2)COC(=O)[C@]3(O)CC ; CCc1c2c(nc3ccc(OC(=O)[C@@H](C)c4ccc(CC(C)C)cc4)cc13)-c1cc3c(c(=O)n1C2)COC(=O)[C@]3(O)CC ; CCc1c2c(nc3ccc(OC(=O)[C@H](C)c4ccc(CC(C)C)cc4)cc13)-c1cc3c(c(=O)n1C2)COC(=O)[C@]3(O)CC]

In yet another aspect, the invention generally relates to a compound having the structural formula of:

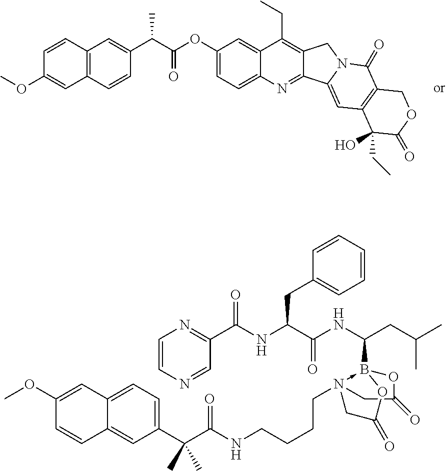

or a pharmaceutically acceptable form or an isotope derivative thereof.

In yet another aspect, the invention generally relates to a compound having the structural formula of (III):

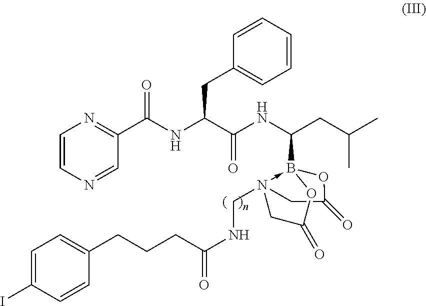

wherein n is an intergern selected from 2-8 (i.e., 2, 3, 4, 5, 6, 7 or 8),
or a pharmaceutically acceptable form or an isotope derivative thereof.

In certain embodiments, the compound of formula (III) is selected from:

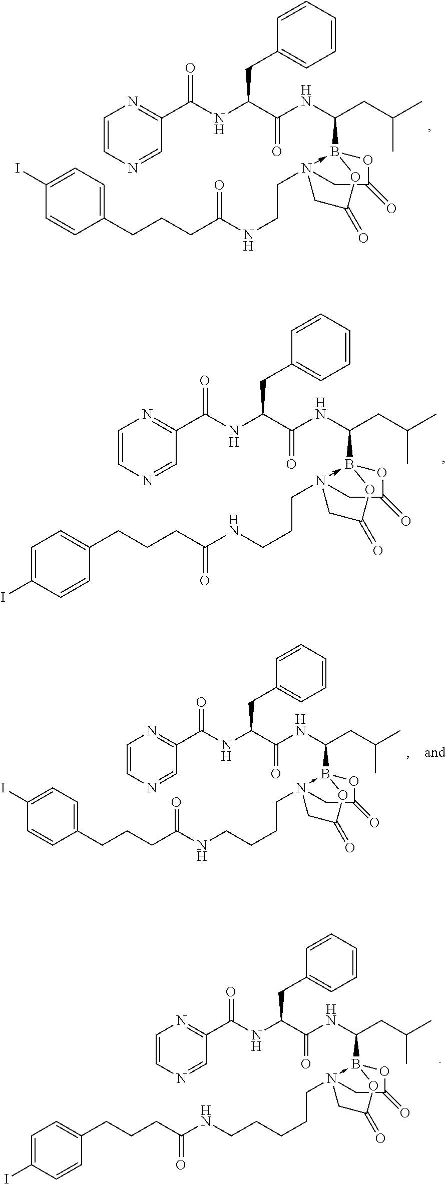 [SMILES: CC(C)C[C@H](NC(=O)[C@H](Cc1ccccc1)NC(=O)c1cnccn1)[B-]12OC(=O)C[N+]1(CCNC(=O)CCCc1ccc(I)cc1)CC(=O)O2 ; CC(C)C[C@H](NC(=O)[C@H](Cc1ccccc1)NC(=O)c1cnccn1)[B-]12OC(=O)C[N+]1(CCCNC(=O)CCCc1ccc(I)cc1)CC(=O)O2 ; CC(C)C[C@H](NC(=O)[C@H](Cc1ccccc1)NC(=O)c1cnccn1)[B-]12OC(=O)C[N+]1(CCCCNC(=O)CCCc1ccc(I)cc1)CC(=O)O2 ; CC(C)C[C@H](NC(=O)[C@H](Cc1ccccc1)NC(=O)c1cnccn1)[B-]12OC(=O)C[N+]1(CCCCCNC(=O)CCCc1ccc(I)cc1)CC(=O)O2]

In yet another aspect, the invention generally relates to a compound having the structural formula of (IV).

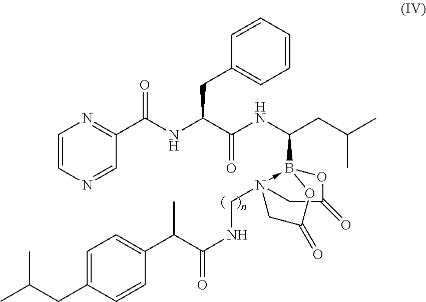

wherein n is an intergern selected from 2-10 (e.g., 2, 3, 4, 5, 6, 7, 8, 9 or 10),
or a pharmaceutically acceptable form or an isotope derivative thereof.

In certain embodiments, the compound of formula (IV) is selected from:

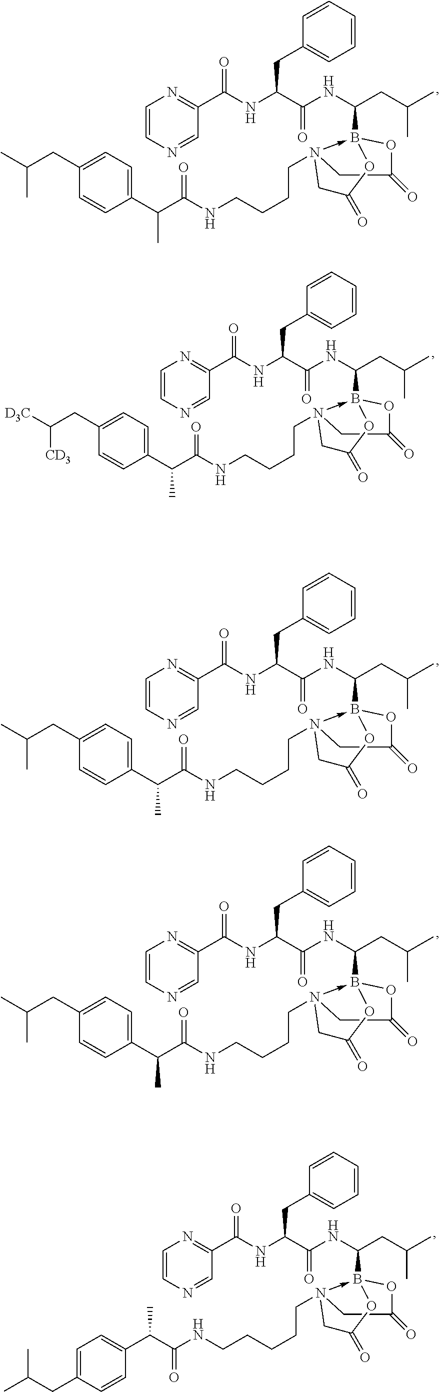

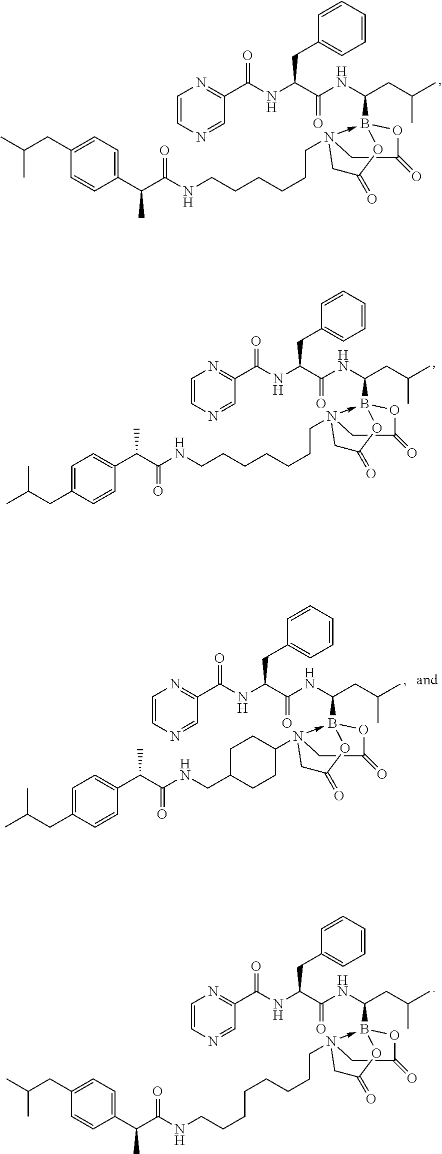

In yet another aspect, the invention generally relates to a compound having the structural formula of (V):

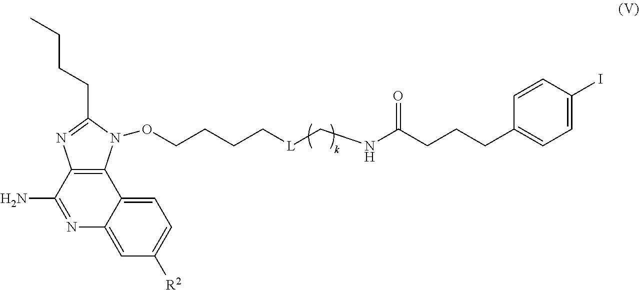

wherein
R² is H or (═O)(R³)₂ and R³ is alkyl,
L is a single bond or a group selected from:

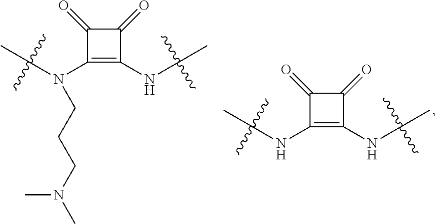 [SMILES: *Nc1c(N(*)CCCN(C)C)c(=O)c1=O ; *Nc1c(N*)c(=O)c1=O]

k is an integer selected from 0-4 (e.g., 0, 1, 2, 3 or 4), or a pharmaceutically acceptable form or an isotope derivative thereof.

In certain embodiments, the compound of formula (V) is selected from:

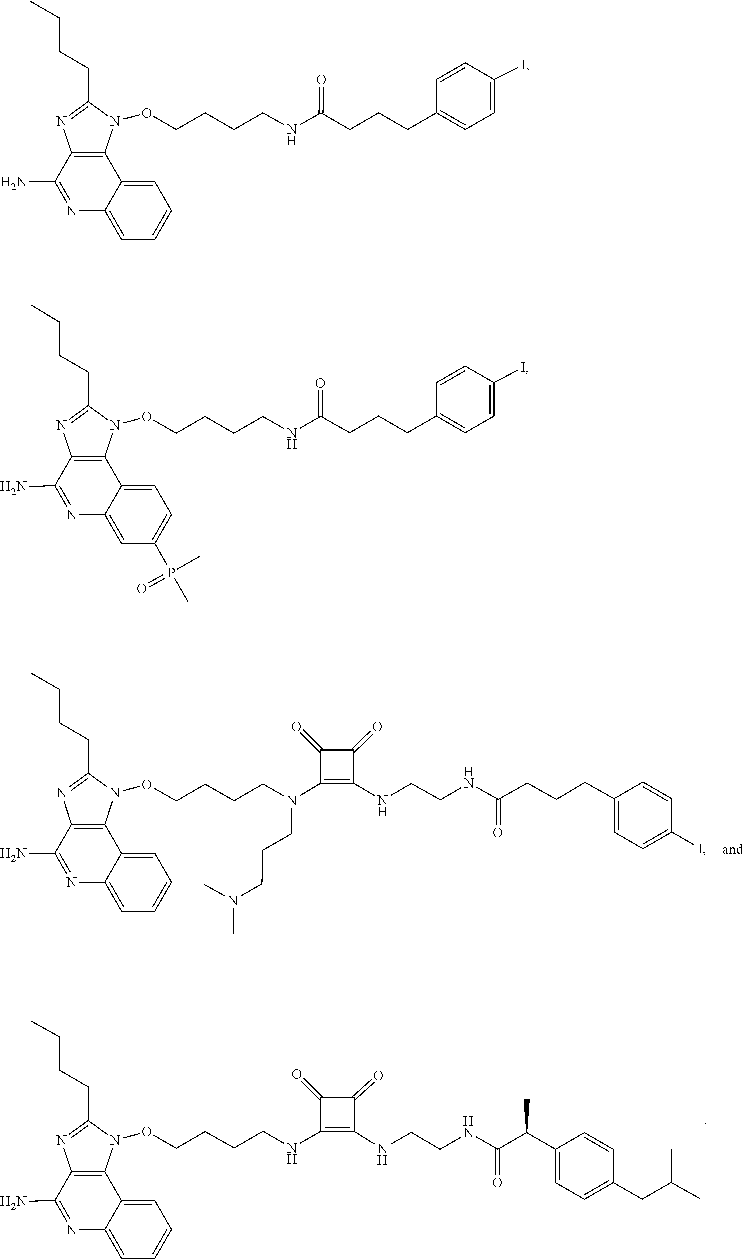 [SMILES: CCCCc1nc2c(N)nc3ccccc3c2n1OCCCCNC(=O)CCCc1ccc(I)cc1 ; CCCCc1nc2c(N)nc3cc(P(C)(C)=O)ccc3c2n1OCCCCNC(=O)CCCc1ccc(I)cc1 ; CCCCc1nc2c(N)nc3ccccc3c2n1OCCCCN(CCCN(C)C)c1c(NCCNC(=O)CCCc2ccc(I)cc2)c(=O)c1=O ; "and" ; CCCCc1nc2c(N)nc3ccccc3c2n1OCCCCNc1c(NCCNC(=O)[C@@H](C)c2ccc(CC(C)C)cc2)c(=O)c1=O]

In yet another aspect, the invention generally relates to a compound having the structural formula of (VI):

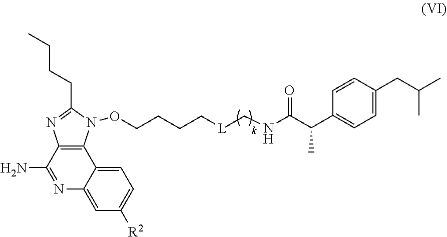

wherein
R² is H or (═O)(R³)₂ and R³ is alkyl,
L is a single bond or a group selected from:

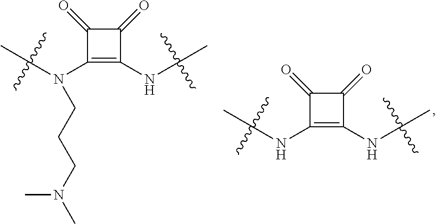 [SMILES: *Nc1c(N(*)CCCN(C)C)c(=O)c1=O ; *Nc1c(N*)c(=O)c1=O]

and
k is an integer selected from 0-4 (e.g., 0, 1, 2, 3 or 4), or a pharmaceutically acceptable form or an isotope derivative thereof.

In certain embodiments, the compound of formula (VI) has the structure:

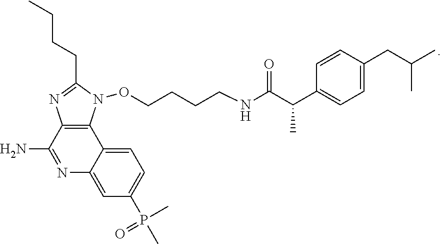 [SMILES: CCCCc1nc2c(N)nc3cc(P(C)(C)=O)ccc3c2n1OCCCCNC(=O)[C@@H](C)c1ccc(CC(C)C)cc1]

In yet another aspect, the invention generally relates to a compound having the structural formula:

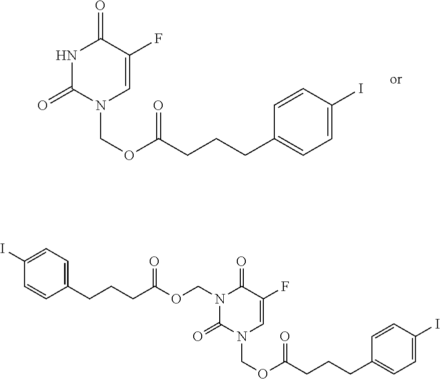 [SMILES: O=C(CCCc1ccc(I)cc1)OCn1cc(F)c(=O)[nH]c1=O ; O=C(CCCc1ccc(I)cc1)OCn1cc(F)c(=O)n(COC(=O)CCCc2ccc(I)cc2)c1=O]

or a pharmaceutically acceptable form or an isotope derivative thereof.

In yet another aspect, the invention generally relates to a compound having the structural formula:

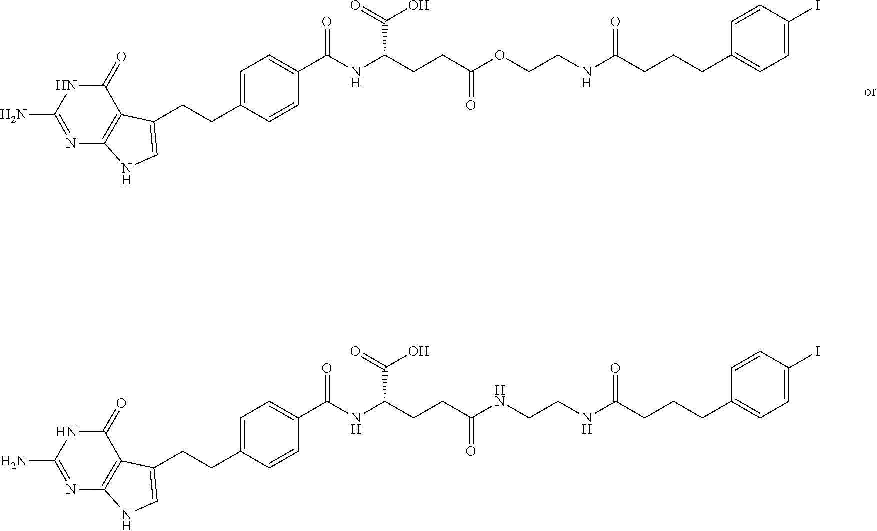 [SMILES: Nc1nc2[nH]cc(CCc3ccc(C(=O)N[C@@H](CCC(=O)OCCNC(=O)CCCc4ccc(I)cc4)C(=O)O)cc3)c2c(=O)[nH]1 ; Nc1nc2[nH]cc(CCc3ccc(C(=O)N[C@@H](CCC(=O)NCCNC(=O)CCCc4ccc(I)cc4)C(=O)O)cc3)c2c(=O)[nH]1]

or a pharmaceutically acceptable form or an isotope derivative thereof.

In yet another aspect, the invention generally relates to a compound having the structural formula:

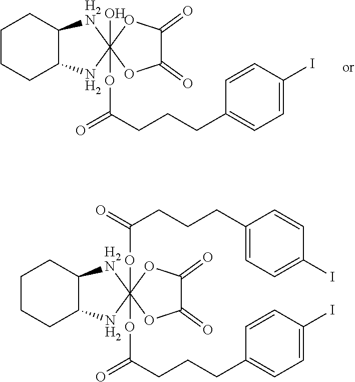

or a pharmaceutically acceptable form or an isotope derivative thereof.

In yet another aspect, the invention generally relates to a compound having the structural formula:

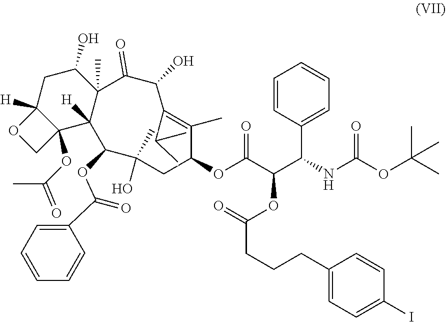

or a pharmaceutically acceptable form or an isotope derivative thereof.

In yet another aspect, the invention generally relates to a compound having the structural formula:

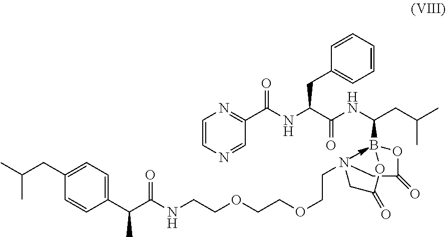 [SMILES: CC(C)Cc1ccc([C@H](C)C(=O)NCCOCCOCCN2CC(=O)OB([C@H](CC(C)C)NC(=O)[C@H](Cc3ccccc3)NC(=O)c3cnccn3)OC(=O)C2)cc1]

or a pharmaceutically acceptable form or an isotope derivative thereof.

In yet another aspect, the invention generally relates to a compound having the structural formula:

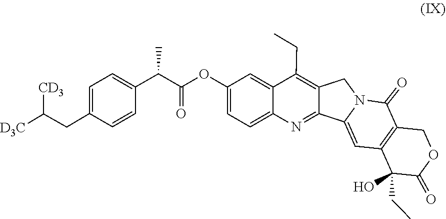 [SMILES: [2H]C([2H])([2H])C(Cc1ccc([C@H](C)C(=O)Oc2ccc3nc4c(c(CC)c3c2)Cn2c-4cc3c(c2=O)COC(=O)[C@]3(O)CC)cc1)C([2H])([2H])[2H]]

or a pharmaceutically acceptable form or an isotope derivative thereof.

In yet another aspect, the invention generally relates to a pharmaceutical composition comprising a compound disclosed herein and a pharmaceutically acceptable excipient, carrier, or diluent.

In certain embodiments, the pharmaceutical composition of the invention is effective to treat or reduce cancer, or a related disease or condition.

In yet another aspect, the invention generally relates to a unit dosage form comprising a pharmaceutical composition comprising a compound disclosed herein.

In yet another aspect, the invention generally relates to a method for treating or reducing a disease or condition, comprising administering to a subject in need thereof a pharmaceutical composition comprising a compound disclosed herein and a pharmaceutically acceptable excipient, carrier, or diluent.

In certain embodiments, the disease or condition is cancer, or a related disease or condition thereof.

In yet another aspect, the invention generally relates to use of a compound disclosed herein for treating or reducing a disease or condition, for example, cancer.

In yet another aspect, the invention generally relates to use of a compound disclosed herein, and a pharmaceutically acceptable excipient, carrier, or diluent, in preparation of a medicament for treating or reducing a disease or condition, for example, cancer.

Compositions of the present invention are administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention include aqueous or oleaginous suspension. These suspensions are formulated according to techniques known in the art using suitable dispersing or wetting agents and suspending agents. The sterile injectable preparation is also a sterile injectable solution or suspension in a non-toxic parenterally acceptable diluent or solvent, for example as a solution in 1,3-butanediol. Among the acceptable vehicles and solvents that are employed are water, Ringer's solution and isotonic sodium chloride solution. In addition, sterile, fixed oils are conventionally employed as a solvent or suspending medium.

For this purpose, any bland fixed oil employed includes synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms are also be used for the purposes of formulation.

Pharmaceutically acceptable compositions of this invention are orally administered in any orally acceptable dosage form. Exemplary oral dosage forms are capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents are optionally also added.

Alternatively, pharmaceutically acceptable compositions of this invention are administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention are also administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches are also used.

For topical applications, provided pharmaceutically acceptable compositions are formulated in a suitable ointment containing the active component suspended or dissolved in one or more carriers. Exemplary carriers for topical administration of compounds of this are mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions of this invention are optionally administered by nasal aerosol or inhalation. Such compositions are prepared according to techniques well-known in the art of pharmaceutical formulation and are prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that are optionally combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### EXAMPLES

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

Compound numbers utilized in the Examples below correspond to compound numbers set forth supra.

¹H was recorded at 400 MHz on a Varian Mercury 400 spectrometer. ¹³C NMR was recorded at 100 MHz. Proton chemical shifts were internally referenced to the residual proton resonance in CDCl3 (7.26 ppm). Carbon chemical shifts were internally referenced to the deuterated solvent signals in CDCl3 (77.20 ppm).

LC-MS spectra were recorded on a Shimadzu LC-MS2020 using Agilent C18 column (Eclipse XDB-C18, 5 μm, 2.1×50 mm) with flow rate of 1 mL/min. Mobile phase A: 0.1% of formic acid in water; mobile phase B: 0.1% of formic acid in acetonitrile. A general gradient method was used.

| Time (min) | A | B |
|---|---|---|
| 0 | 95 | 5 |
| 3 | 0 | 100 |
| 4 | 0 | 100 |
| 4.05 | 95 | 5 |

Analytical HPLC was performed on Agilent 1200 HPLC with a Zorbax Eclipse XDB C18 column (2.1×150 mm) with flow rate of 1 mL/min. Mobile phase A: 0.1% of TFA in water; mobile phase B: 0.1% of TFA in acetonitrile. A general method with the following gradient was used.

| Time (min) | Mobile Phase A | Mobile Phase B |
|---|---|---|
| 0 | 95 | 5 |
| 15 | 0 | 100 |
| 16 | 0 | 100 |
| 16.5 | 95 | 5 |
| 16.5 | | stop |

Preparative HPLC was performed on Varian ProStar using Hamilton C18 PRP-1 column (15×250 mm) with flow rate of 20 mL/min. Mobile phase A: 0.1% of TFA in water; mobile phase B: 0.1% of TFA in acetonitrile. A typical gradient method was used.

| Time (min) | Mobile Phase A | Mobile Phase B |
|---|---|---|
| 0 | 90 | 10 |
| 30 | 30 | 70 |
| 35 | 10 | 100 |
| 40 | 90 | 10 |
| 45 | | stop |

### Example 1

### (R)-4,11-diethyl-4-methyl-3,14-dioxo-3,4,12,14-tetrahydro-1H-pyrano [3′,4′:6,7]indolizino[1,2-b]quinolin-9-yl 4-(4-iodophenyl)butanoate (1)

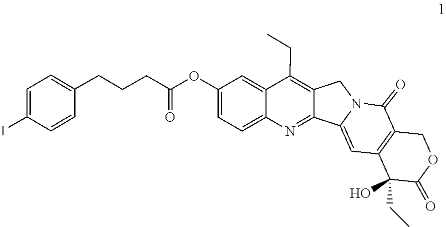 [SMILES: CCc1c2c(nc3ccc(OC(=O)CCCc4ccc(I)cc4)cc13)-c1cc3c(c(=O)n1C2)COC(=O)[C@]3(O)CC]

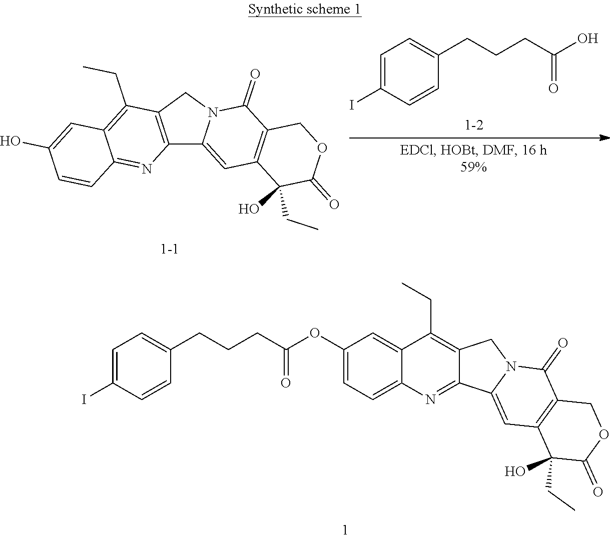 [SMILES: CCc1c2c(nc3ccc(O)cc13)-c1cc3c(c(=O)n1C2)COC(=O)[C@]3(O)CC ; O=C(O)CCCc1ccc(I)cc1 ; CCc1c2c(nc3ccc(OC(=O)CCCc4ccc(I)cc4)cc13)-c1cc3c(c(=O)n1C2)COC(=O)[C@]3(O)CC]

Step 1: To a solution of compound 1-2 (600 mg, 2.06 mmol) in DMF (10 mL) was added EDCI (800 mg, 4.18 mmol) and HOBt (190 mg, 1.40 mmol). After stirring for 5 minutes, compound 1-1 (SN-38, 500 mg, 1.27 mmol) was added, and the mixture was stirred at room temperature for 16 hours. After completion of the reaction, water (100 mL) was added and the mixture was stirred at room temperature for 30 min. A white precipitation was formed completely. The resulting mixture was filtered and washed with water to give an off-white solid. The crude product was then triturated by acetonitrile for one hour and filtered. The filter cake was washed with acetonitrile and dried to give the title compound 1 (505 mg, 59% yield) as an off-white solid. ¹H NMR (400 MHz, DMSO-d6) δ 8.19 (d, J=9.1 Hz, 1H), 8.04-7.94 (m, 1H), 7.66 (td, J=10.1, 9.2, 4.6 Hz, 3H), 7.32 (s, 1H), 7.10 (d, J=7.8 Hz, 2H), 6.53 (s, 1H), 5.44 (s, 2H), 5.32 (s, 2H), 3.17 (q, J=7.6 Hz, 2H), 2.76-2.62 (m, 4H), 1.98 (p, J=7.4 Hz, 2H), 1.87 (hept, J=7.0 Hz, 2H), 1.28 (t, J=7.5 Hz, 3H), 0.88 (t, J=7.3 Hz, 3H). LCMS: m/z calculated for C₃₂H₂₉IN₂O₆: 664.50; found: 665.2 [M+H]⁺.

### Example 2

### (R)-4,11-diethyl-4-methyl-3,14-dioxo-3,4,12,14-tetrahydro-1H-pyrano [3′,4′:6,7]indolizino[1,2-b]quinolin-9-yl 2-methyl-4-(4-iodophenyl)butanoate (2)

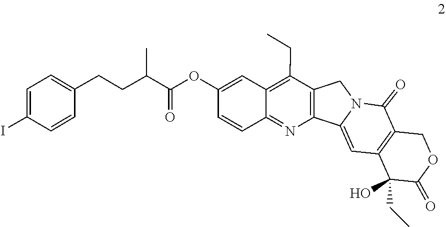 [SMILES: CCc1c2c(nc3ccc(OC(=O)C(C)CCc4ccc(I)cc4)cc13)-c1cc3c(c(=O)n1C2)COC(=O)[C@]3(O)CC]

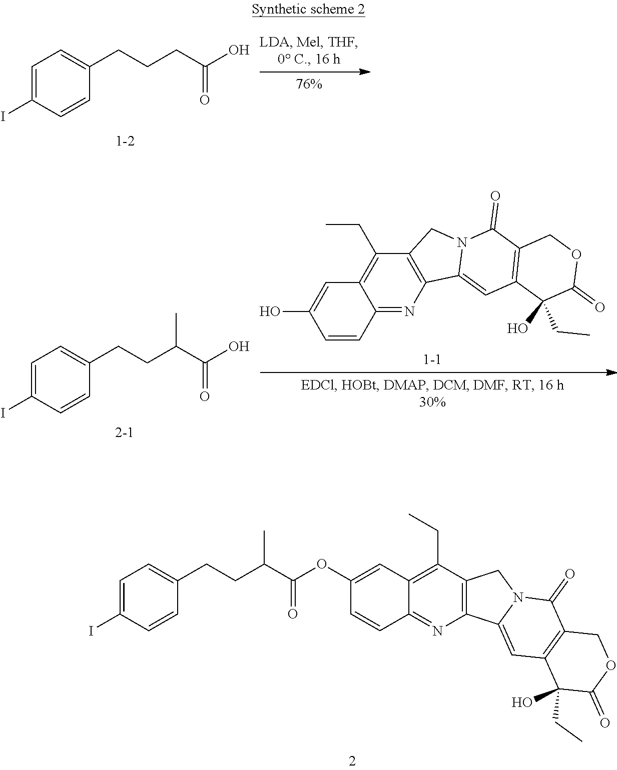 [SMILES: O=C(O)CCCc1ccc(I)cc1 ; CC(CCc1ccc(I)cc1)C(=O)O ; CCc1c2c(nc3ccc(O)cc13)-c1cc3c(c(=O)n1C2)COC(=O)[C@]3(O)CC ; CCc1c2c(nc3ccc(OC(=O)C(C)CCc4ccc(I)cc4)cc13)-c1cc3c(c(=O)n1C2)COC(=O)[C@]3(O)CC]

Step 1: LDA (2 M, 2.5 mL, 5 mmol) was added dropwise to a stirring solution of compound 1-2 (500 mg, 1.72 mmol) in anhydrous THF (10 mL) at 0° C. under nitrogen atmosphere. After addition, the mixture was stirred at 0° C. for 30 min. Mel (538 mg, 3.79 mmol) was added dropwise and the reaction was allowed to stir at room temperature for 16 h. After completion of the reaction, the mixture was quenched with saturated NH₄Cl (30 mL) solution and extracted with EtOAc (30 mL×3). The organic layers were washed with brine, dried over anhydrous Na₂SO₄, filtered and concentrated. The crude product was purified by silica gel column (eluted with 30% EtOAc in Petroleum ether) to give the title compound 2-1 (400 mg, yield 76%).

Step 2: A mixture of compound 2-1 (390 mg, 1.24 mmol), compound 1-1 (194 mg, 0.496 mmol), HOBt (335 mg, 2.48 mmol), DMAP (30 mg, 0.248 mmol) and EDCI (950 mg, 4.97 mmol) in DMF (5 mL) and DCM (5 mL) was stirred at room temperature for 16 h. After completion of the reaction, the mixture was partitioned between EtOAc (100 mL) and water (100 mL). The organic layer was washed with brine, dried over anhydrous Na₂SO₄, filtered and concentrated. The crude product was purified by silica gel column (eluted with 5% MeOH in DCM) to afford the title compound 2 (265 mg, yield 30%).

### Example 3

### (S)-4,11-diethyl-4-hydroxy-3,14-dioxo-3,4,12,14-tetrahydro-1H-pyrano [3′,4′:6,7]indolizino[1,2-b]quinolin-9-yl(4-iodophenethyl)carbamate (3)

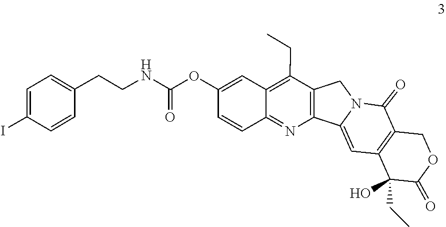 [SMILES: CCc1c2c(nc3ccc(OC(=O)NCCc4ccc(I)cc4)cc13)-c1cc3c(c(=O)n1C2)COC(=O)[C@]3(O)CC]

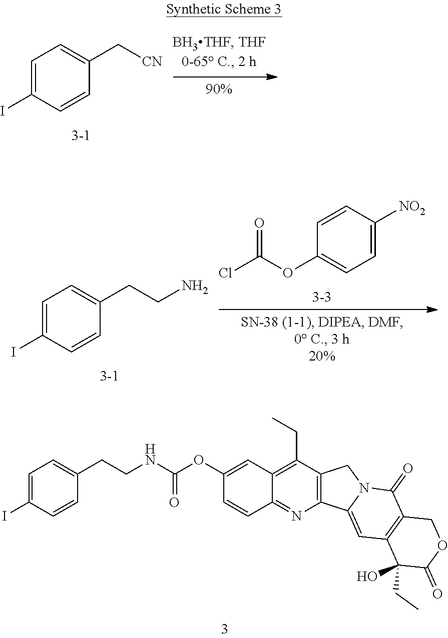 [SMILES: N#CCc1ccc(I)cc1 ; O=C(Cl)Oc1ccc([N+](=O)[O-])cc1 ; NCCc1ccc(I)cc1 ; CCc1c2c(nc3ccc(OC(=O)NCCc4ccc(I)cc4)cc13)-c1cc3c(c(=O)n1C2)COC(=O)[C@]3(O)CC]

Step 1: To a solution of compound 3-1 (1.0 g, 4.11 mmol) in anhydrous THE (10 mL) at 0° C. was added dropwise a solution of borane-tetrahydrofuran complex (1 M in THF, 18.5 mL) under nitrogen atmosphere. The reaction mixture was stirred at 65° C. for 2 h. After completion of the reaction, the mixture was cooled to 0° C. and quenched by addition of 6 N HCl (2 mL). The resulting mixture was then basified with 1 N NaOH solution and extracted with DCM (50 mL×2). The combined organic layers were dried over anhydrous Na₂SO₄, filtered and concentrated to afford the title compound 3-2 (1.0 g, yield 90%) as a colorless oil, which was used directly at the next step without further purification.

Step 2: Compound 3-3 (345 mg, 1.71 mmol) was dissolved in DCM (5 mL) and cooled to 0° C. To this was added slowly a solution of compound 3-2 (210 mg, 0.85 mmol) and TEA (257 mg, 2.55 mmol) in DMSO (2 mL). The mixture was stirred at 0° C. for 1 h. SN-38 (1-1, 330 mg, 0.84 mmol) and TEA (100 mg, 0.99 mmol) were added. Then the mixture was stirred at room temperature for 3 h. After completion of the reaction, the mixture was partitioned between EtOAc (50 mL) and water (50 mL). The organic layer was washed with brine, dried over anhydrous Na₂SO₄, filtered and concentrated. The crude product was purified by silica gel column (eluted with 5% MeOH in DCM) to afford the title compound 3 (120 mg, yield 20%).

### Example 4

### (S)-4,11-diethyl-4-hydroxy-3,14-dioxo-3,4,12,14-tetrahydro-1H-pyrano[3′,4′:6,7]indolizino[1,2-b]quinolin-9-yl(4-iodophenethyl)(methyl)carbamate (4)

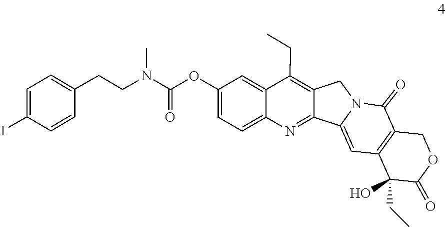 [SMILES: CCc1c2c(nc3ccc(OC(=O)N(C)CCc4ccc(I)cc4)cc13)-c1cc3c(c(=O)n1C2)COC(=O)[C@]3(O)CC]

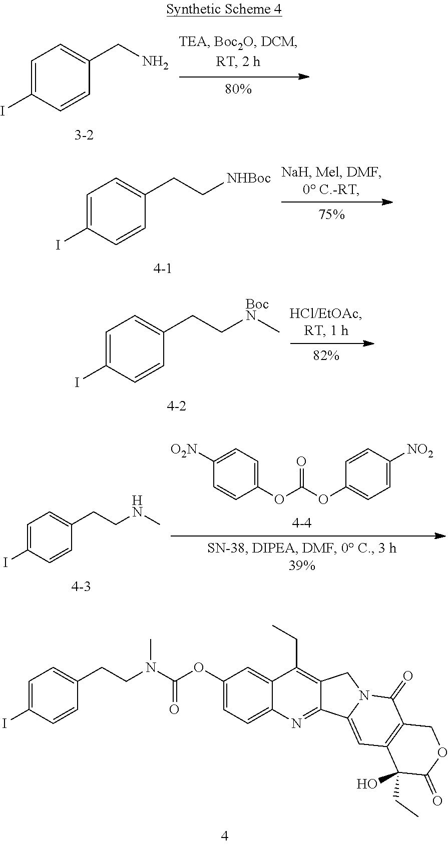 [SMILES: NCc1ccc(I)cc1 ; CC(C)(C)OC(=O)NCCc1ccc(I)cc1 ; CN(CCc1ccc(I)cc1)C(=O)OC(C)(C)C ; O=C(Oc1ccc([N+](=O)[O-])cc1)Oc1ccc([N+](=O)[O-])cc1 ; CNCCc1ccc(I)cc1 ; CCc1c2c(nc3ccc(OC(=O)N(C)CCc4ccc(I)cc4)cc13)-c1cc3c(c(=O)n1C2)COC(=O)[C@]3(O)CC]

Step 1: A mixture of compound 3-2 (1.0 g, 4.04 mmol), BOC₂O (1.7 g, 7.79 mmol) and TEA (830 mg, 8.20 mmol) in DCM (10 mL) was stirred at room temperature for 2 h. After completion of the reaction, the mixture was concentrated. The residue was purified by silica gel column (elute with 10% EtOAc in petroleum ether) to afford the title compound 4-1 (1.1 g, yield 80%) as a white solid. LCMS: m/z calculated for C₁₃H₁₈INO₂: 347.20; found: 348.21 [M+H]⁺.

Step 2: NaH (60% in mineral oil, 450 mg, 11.25 mmol) was added in several portions to a solution of compound 4-1 (1.3 g, 3.74 mmol) in anhydrous DMF (5 mL) at 0° C. After stirring for 1 h, Mel (1.6 g, 11.27 mmol) was added to the mixture in one portion. Then the mixture was stirred at room temperature for 16 h. After completion of the reaction, the mixture was quenched by addition of ice water and partitioned between EtOAc (100 mL) and water (50 mL). The organic layer was washed with brine, dried over anhydrous Na₂SO₄ and concentrated. The crude product was purified by silica gel column (elute with 5% EtOAc in petroleum ether) to afford the title compound 4-2 (1.02 g, yield 75%) as a white solid. LCMS: m/z calculated for C₁₄H₂₀INO₂: 361.22; found: 306.04 [M-^{t}Bu+H]⁺.

Step 3: A mixture of compound 4-2 (1.02 g, 2.82 mmol) in EtOAc (5 mL) and a solution of HCl in EtOAc (4 N, 5 mL) was stirred at room temperature for 1 h. After completion of the reaction, a white precipitation was formed completely. The white precipitation was filtered and washed with EtOAc to afford the title compound 4-3 (610 mg, yield 82%), which was used directly at the next step without further purification.

Step 4: A suspension of SN-38 (1-1, 250 mg, 0.637 mmol), DIPEA (170 mg, 1.31 mmol) and compound 4-4 (215 mg, 0.706 mmol) in anhydrous DMF (5 mL) was stirred at 0° C. under nitrogen atmosphere for 1 h. Then the mixture was treated with a solution of compound 4-3 (227 mg, 0.763 mmol) and DIPEA (200 mg, 1.55 mmol). After stirring for an additional 2 h. The resulting mixture was diluted with EtOAc (50 mL) and washed with water. The organic layer was washed with brine, dried over anhydrous Na₂SO₄ and concentrated. The crude product was purified by silica gel column (elute with 2% MeOH in DCM) to afford 4 (170 mg, yield 39%) as a light-yellow solid. ¹H NMR (400 MHz, Chloroform-d) δ 8.37-8.16 (m, 1H), 7.84 (d, J=2.8 Hz, 1H), 7.69 (dd, J=5.6, 3.0 Hz, 3H), 7.61-7.48 (m, 1H), 7.35-7.28 (m, 1H), 7.16-6.97 (m, 2H), 5.79 (dd, J=16.3, 2.8 Hz, 1H), 5.49-5.26 (m, 3H), 3.91 (s, 1H), 3.80 (t, J=7.1 Hz, 1H), 3.66 (t, J=7.8 Hz, 1H), 3.53 (d, J=2.8 Hz, 1H), 3.19 (d, J=7.4 Hz, 1H), 3.16 (d, J=2.8 Hz, 1H), 3.09 (d, J=2.8 Hz, 1H), 3.01-2.91 (m, 2H), 1.93 (ddd, J=15.0, 7.4, 4.0 Hz, 2H), 1.48-1.38 (m, 3H), 1.13-1.02 (m, 3H). LCMS: m/z calculated for C₃₂H₃₀IN₃O₆: 679.51; found: 680.31 [M+H]⁺.

### Example 5

### (S)-4,11-diethyl-4-hydroxy-3,14-dioxo-3,4,12,14-tetrahydro-1H-pyrano [3′,4′:6,7]indolizino[1,2-b]quinolin-9-yl 2-(4-isobutylphenyl)propanoate (5)

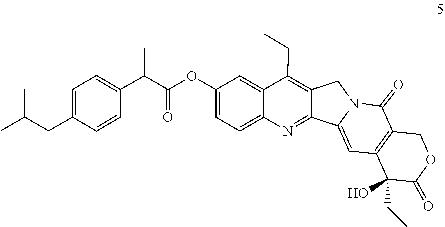 [SMILES: CCc1c2c(nc3ccc(OC(=O)C(C)c4ccc(CC(C)C)cc4)cc13)-c1cc3c(c(=O)n1C2)COC(=O)[C@]3(O)CC]

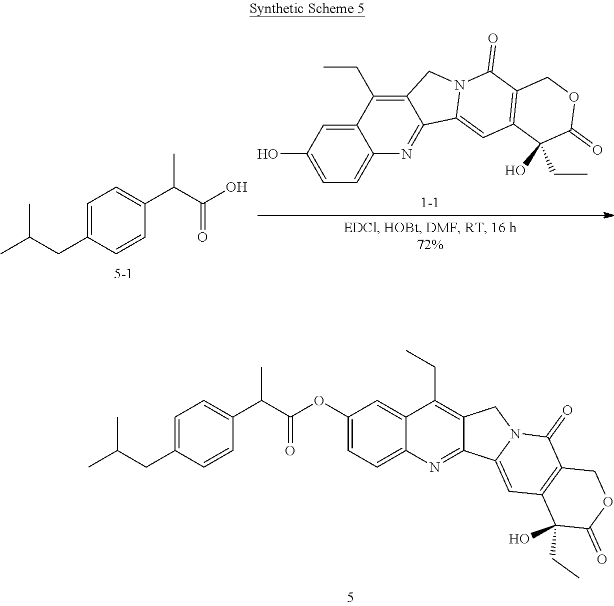 [SMILES: CC(C)Cc1ccc(C(C)C(=O)O)cc1 ; CCc1c2c(nc3ccc(O)cc13)-c1cc3c(c(=O)n1C2)COC(=O)[C@]3(O)CC ; CCc1c2c(nc3ccc(OC(=O)C(C)c4ccc(CC(C)C)cc4)cc13)-c1cc3c(c(=O)n1C2)COC(=O)[C@]3(O)CC]

Step 1: A mixture of compound 5-1 (236.6 mg, 1.15 mmol), EDCI (438.1 mg, 2.29 mmol) and HOBt (103.2 mg, 0.76 mmol) in DMF (3 mL) was stirred at room temperature for 5 min. Compound 1-1 (300 mg, 0.76 mmol) was added and the mixture was stirred at room temperature for 15 h. The mixture was partitioned between EtOAc (30 mL) and water (30 mL). The organic layer was washed with brine, dried over anhydrous Na₂SO₄, filtered and concentrated. The crude product was purified by silica gel column (eluted with 2% MeOH in DCM) to afford the title compound 5 (324 mg, yield 72%) as a white solid. ¹H NMR (500 MHz, DMSO-d₆) δ 8.18 (d, J=9.1 Hz, 1H), 7.89 (d, J=2.4 Hz, 1H), 7.50 (dd, J=9.2, 2.5 Hz, 1H), 7.39-7.36 (m, 2H), 7.32 (s, 1H), 7.22-7.19 (m, 2H), 6.53 (s, 1H), 5.43 (s, 2H), 5.31 (s, 2H), 4.15 (q, J=7.1 Hz, 1H), 3.15 (q, J=7.6 Hz, 2H), 2.46 (d, J=7.2 Hz, 2H), 1.92-1.81 (m, 3H), 1.57 (d, J=7.1 Hz, 3H), 1.27 (t, J=7.6 Hz, 3H), 0.88 (dd, J=7.1, 2.3 Hz, 9H). LCMS: m/z calculated for C₃₅H₃₆N₂O₆: 580.68; found: 581.47 [M+H]⁺.

### Example 6

### (S)-4,11-diethyl-4-hydroxy-3,14-dioxo-3,4,12,14-tetrahydro-1H-pyrano [3′,4′:6,7]indolizino[1,2-b]quinolin-9-yl(S)-2-(6-methoxynaphthalen-2-yl)propanoate (6)

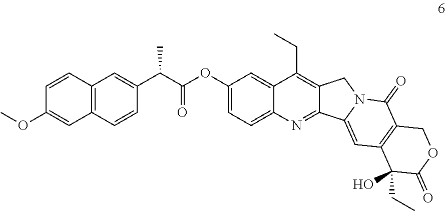 [SMILES: CCc1c2c(nc3ccc(OC(=O)[C@@H](C)c4ccc5cc(OC)ccc5c4)cc13)-c1cc3c(c(=O)n1C2)COC(=O)[C@]3(O)CC]

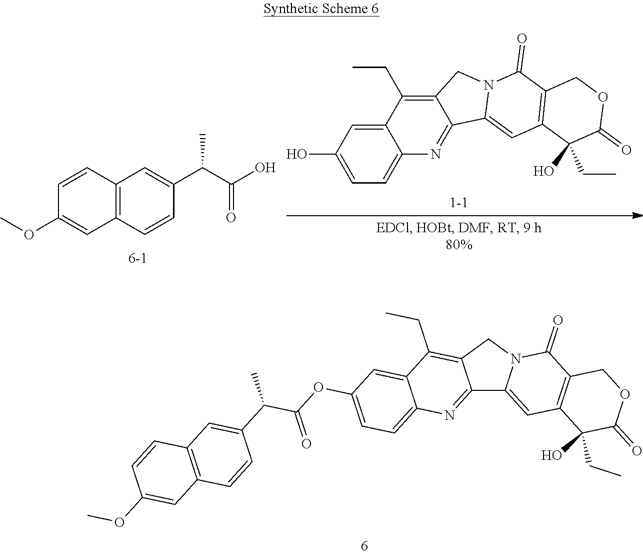

Step 1: A mixture of compound 6-1 (176.0 mg, 0.76 mmol), SN-38 (1-1) (150 mg, 0.38 mmol), HOBt (51.6 mg, 0.38 mmol) and EDCI (219.0 mg, 1.15 mmol) in DMF (3 mL) was stirred at room temperature for 9 h. After completion of the reaction, the reaction mixture was diluted with EtOAc and washed with water. The organic layer was washed with brine, dried over anhydrous Na₂SO₄ and concentrated. The crude product was purified by column (eluted with 2% MeOH in dichloromethane) to afford 6 (185 mg, yield 80%) as a white solid. ¹H NMR (400 MHz, DMSO-d₆) δ 8.16 (d, J=9.1 Hz, 1H), 7.91 (dd, J=4.4, 2.1 Hz, 2H), 7.87 (d, J=8.7 Hz, 2H), 7.58 (dd, J=8.5, 1.8 Hz, 1H), 7.52 (dd, J=9.1, 2.5 Hz, 1H), 7.34 (d, J=2.6 Hz, 1H), 7.30 (s, 1H), 7.19 (dd, J=9.0, 2.5 Hz, 1H), 6.53 (s, 1H), 5.43 (s, 2H), 5.30 (s, 2H), 4.32 (q, J=7.1 Hz, 1H), 3.87 (s, 3H), 3.14 (q, J=7.5 Hz, 2H), 1.85 (dq, J=14.2, 7.1 Hz, 2H), 1.66 (d, J=7.1 Hz, 3H), 1.25 (t, J=7.6 Hz, 3H), 0.87 (t, J=7.3 Hz, 3H). LCMS: m/z calculated for C₃₆H₃₂N₂O₇: 604.66; found: 605.36. [M+H]⁺. TLC (DCM:MeOH/40:1): R_{f} (compound 6-1)=0.5; R_{f} (compound 1-1)=0.3; R_{f} (6)=0.37.

### Example 7

### (S)-4,11-diethyl-4-hydroxy-3,14-dioxo-3,4,12,14-tetrahydro-1H-pyrano [3′,4′:6,7]indolizino[1,2-b]quinolin-9-yl(R)-2-(4-isobutylphenyl)propanoate (7)

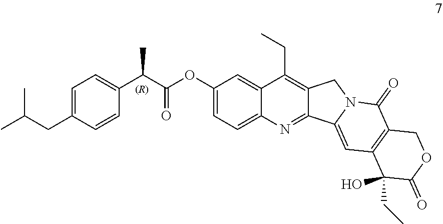

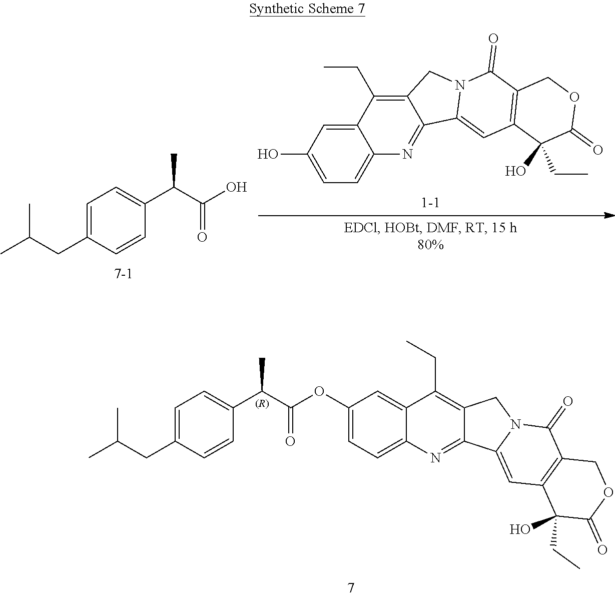

Step 1: A mixture of compound 7-1 (107 mg, 0.51 mmol), EDCI (198.2 mg, 1.04 mmol) and HOBt (46.7 mg, 0.35 mmol) in DMF (3 mL) was stirred at room temperature for 5 min. Compound 1-1 (135.7 mg, 0.35 mmol) was added and the mixture was stirred at room temperature and for 15 h. The reaction mixture was partitioned between EtOAc (20 mL) and water (20 mL). The organic layer was washed with brine, dried over anhydrous Na₂SO₄, filtered and concentrated. The crude product was purified by silica gel column (eluted with 2% MeOH in DCM) to afford the title compound 7 (161 mg, yield 80%) as a white solid. ¹H NMR (500 MHz, DMSO-d₆) δ 8.22-8.11 (m, 1H), 7.93-7.86 (m, 1H), 7.53-7.46 (m, 1H), 7.38 (d, J=7.6 Hz, 2H), 7.32 (d, J=2.3 Hz, 1H), 7.21 (d, J=7.8 Hz, 2H), 6.53 (s, 1H), 5.44 (s, 2H), 5.33 (s, 2H), 4.16 (q, J=7.2 Hz, 1H), 3.16 (p, J=6.6, 6.1 Hz, 2H), 2.47 (d, J=7.2 Hz, 2H), 1.93-1.80 (m, 3H), 1.57 (d, J=7.1 Hz, 3H), 1.27 (t, J=7.6 Hz, 3H), 0.88 (d, J=6.3 Hz, 9H). LCMS: m/z calculated for C₃₅H₃₆N₂O₆: 580.68; found: 581.47 [M+H]⁺.

### Example 8

### (S)-4,11-diethyl-4-hydroxy-3,14-dioxo-3,4,12,14-tetrahydro-1H-pyrano [3′,4′:6,7]indolizino[1,2-b]quinolin-9-yl(S)-2-(4-isobutylphenyl)propanoate (8)

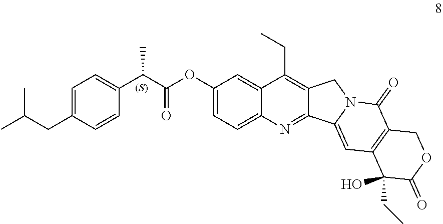

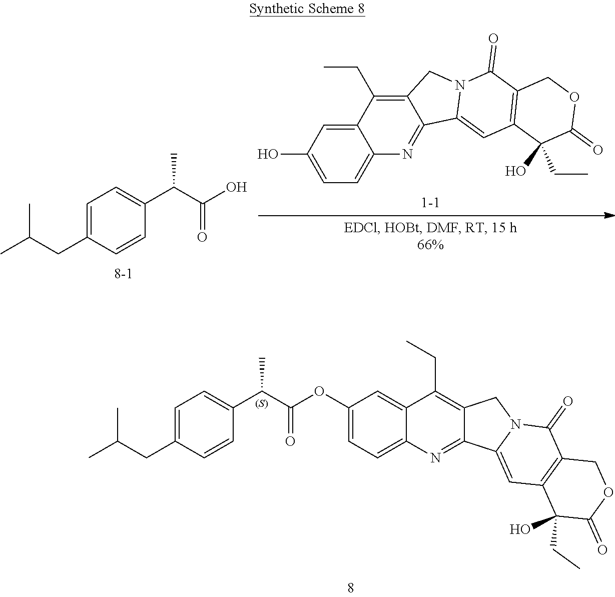

Step 1: A mixture of compound 8-1 (157.7 mg, 0.76 mmol), EDCI (292.0 mg, 1.53 mmol) and HOBt (68.8 mg, 0.51 mmol) in DMF (2 mL) was stirred at room temperature for 5 min. Compound 1-1 (200 mg, 0.51 mmol) was added and the mixture was stirred at room temperature for 15 h. The reaction mixture was partitioned between EtOAc (30 mL) and water (30 mL). The organic layer was washed with brine, dried over anhydrous Na₂SO₄, filtered and concentrated. The crude product was purified by silica gel column (eluted with 2% MeOH in DCM) to afford the title compound 8 (197 mg, yield 66%) as a white solid. ¹H NMR (500 MHz, DMSO-d₆) δ 8.17 (d, J=9.1 Hz, 1H), 7.89 (t, J=2.3 Hz, 1H), 7.50 (dd, J=9.1, 2.5 Hz, 1H), 7.39-7.36 (m, 2H), 7.31 (s, 1H), 7.22-7.19 (m, 2H), 6.52 (s, 1H), 5.43 (s, 2H), 5.31 (s, 2H), 4.15 (q, J=7.1 Hz, 1H), 3.15 (q, J=7.6 Hz, 2H), 2.46 (d, J=7.1 Hz, 2H), 1.92-1.80 (m, 3H), 1.57 (d, J=7.1 Hz, 3H), 1.26 (t, J=7.6 Hz, 3H), 0.87 (dd, J=7.0, 2.8 Hz, 9H). LCMS: m/z calculated for C₃₅H₃₆N₂O₆: 580.68; found: 581.47 [M+H]⁺.

### Example 9

### N-((S)-1-(((R)-1-(6-(2-(4-(4-iodophenyl)butanamido)ethyl)-4,8-dioxo-1,3,6,2-dioxazaborocan-2-yl)-3-methylbutyl)amino)-1-oxo-3-phenylpropan-2-yl)pyrazine-2-carbox amide (9)

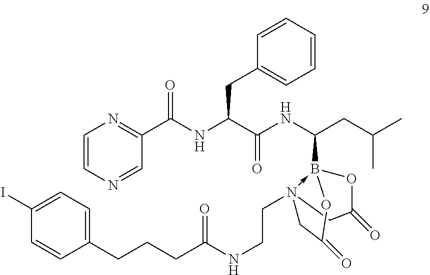 [SMILES: CC(C)C[C@H](NC(=O)[C@H](Cc1ccccc1)NC(=O)c1cnccn1)[B-]12OC(=O)C[N+]1(CCNC(=O)CCCc1ccc(I)cc1)CC(=O)O2]

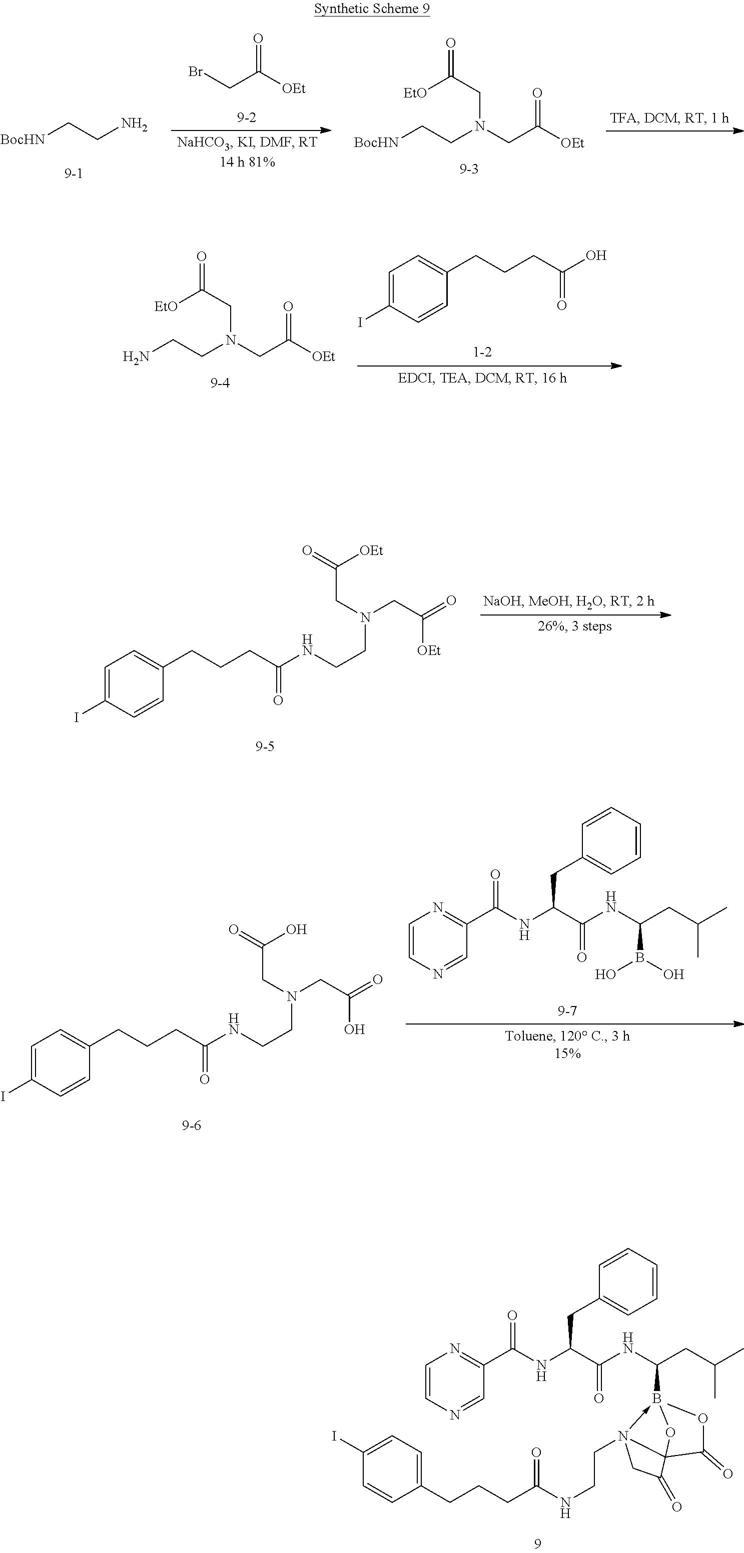

Step 1: To a solution of compound 9-1 (1.0 g, 6.24 mmol) in DMF (25 mL) was added ethyl bromoacetate (9-2, 9.9 g, 59.2 mmol), KI (1.04 g, 6.26 mmol) and NaHCO₃ (5.0 g, 59.5 mmol). The mixture was stirred at room temperature for 14 h. After completion of the reaction, the resulting mixture was diluted with EtOAc (200 mL), washed with water (200 mL×2) and brine (200 mL). The organic layers were dried by anhydrous Na₂SO₄, filtered and concentrated. The residue was purified by silica gel chromatography (eluted with 20% EtOAc in Petroleum ether) to afford the title compound 9-3 (1.7 g, 81% yield) as a colorless oil.

Step 2: A mixture of compound 9-3 (500 mg, 1.50 mmol) and TFA (1 mL) in DCM (4 mL) was stirred at room temperature for 1 h. After completion of the reaction, the resulting mixture was concentrated to give the title compound 9-4 (600 mg) as a colorless oil, which was used at the next step without further purification.

Step 3: Compound 9-4 (600 mg, crude from Step 2) was dissolved in DCM (5 mL) and adjusted pH=8 by adding Et3N. Then the solution was added to a stirring mixture of compound 1-2 (360 mg, 1.24 mmol) and EDCI (431 mg, 2.25 mL) in DCM (10 mL). After stirring for 16 h, the resulting mixture was diluted with DCM (50 mL) and washed with water (50 mL) and 1 M HCl (50 mL×2). The organic layer was dried by anhydrous Na₂SO₄, filtered and concentrated to give the title compound 9-5 (600 mg) as a colorless oil, which was used at the next step without further purification.

Step 4: A mixture of compound 9-5 (600 mg, crude from Step 3) in MeOH (10 mL) was treated with aqueous NaOH (1 M, 3.67 mL, 3.67 mmol), and stirred at room temperature for 2 h. After completion of the reaction, the mixture was concentrated to get rid of the organic solvents. The aqueous solution was washed with EtOAc (50 mL×3) and acidified with 1 M HCl to pH=2 to form a white precipitation. The precipitation was filtered and washed with water to give the title compound 9-6 (180 mg, 26% yield over three steps) as a white solid. LCMS: m/z calculated for C₁₆H₂₁IN₂O₅: 448.26; found: 449.03 [M+H]⁺

Step 5: A mixture of compound 9-6 (180 mg, 0.40 mmol) and compound 9-7 (155 mg, 0.40 mmol) in toluene (18 mL) was stirred at 120° C. for 3 h. After completion of the reaction, the mixture was concentrated. The residue was washed by MTBE and filtered to give a solid. The crude product was re-dissolved in acetonitrile and purified by pre-TLC (SiO₂, DCM/acetonitrile=2/1) to afford the title compound 9 (50.1 mg, 15% yield) as a white solid. ¹H NMR (400 MHz, DMSO-d6) δ 9.09 (s, 1H), 8.90 (d, J=2.5 Hz, 1H), 8.83 (d, J=8.6 Hz, 1H), 8.75 (s, 1H), 8.17 (s, 1H), 7.62 (d, J=7.8 Hz, 2H), 7.42 (d, J=9.7 Hz, 1H), 7.28 (q, J=8.1, 7.4 Hz, 4H), 7.20 (d, J=7.0 Hz, 1H), 7.02 (d, J=7.9 Hz, 2H), 4.74 (q, J=8.2 Hz, 1H), 4.33 (d, J=17.2 Hz, 1H), 4.24-4.11 (m, 2H), 3.79 (d, J=16.8 Hz, 1H), 3.62-3.53 (m, 1H), 3.22 (s, 1H), 3.17 (s, 1H), 3.06 (d, J=8.5 Hz, 2H), 2.51 (s, 1H), 2.17-2.09 (m, 2H), 1.80 (t, J=7.7 Hz, 2H), 1.59 (s, 1H), 1.42 (t, J=12.9 Hz, 1H), 1.25 (d, J=14.1 Hz, 4H), 0.86 (dd, J=13.0, 6.5 Hz, 6H). LCMS: m/z calculated for C₃₅H₄₂BIN₆O₇: 796.47; found: 797.6 [M+H]⁺

### Example 10

### N-((S)-1-(((R)-1-(6-(4-(4-(4-iodophenyl)butanamido)butyl)-4,8-dioxo-1,3,6,2-dioxazaborocan-2-yl)-3-methylbutyl)amino)-1-oxo-3-phenylpropan-2-yl)pyrazine-2-carbox amide (10)

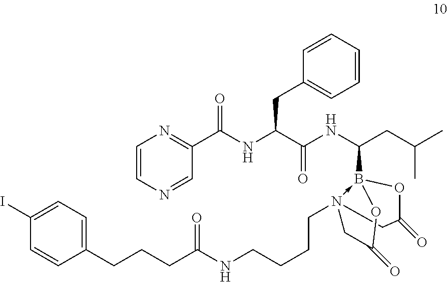 [SMILES: CC(C)C[C@H](NC(=O)[C@H](Cc1ccccc1)NC(=O)c1cnccn1)[B-]12OC(=O)C[N+]1(CCCCNC(=O)CCCc1ccc(I)cc1)CC(=O)O2]

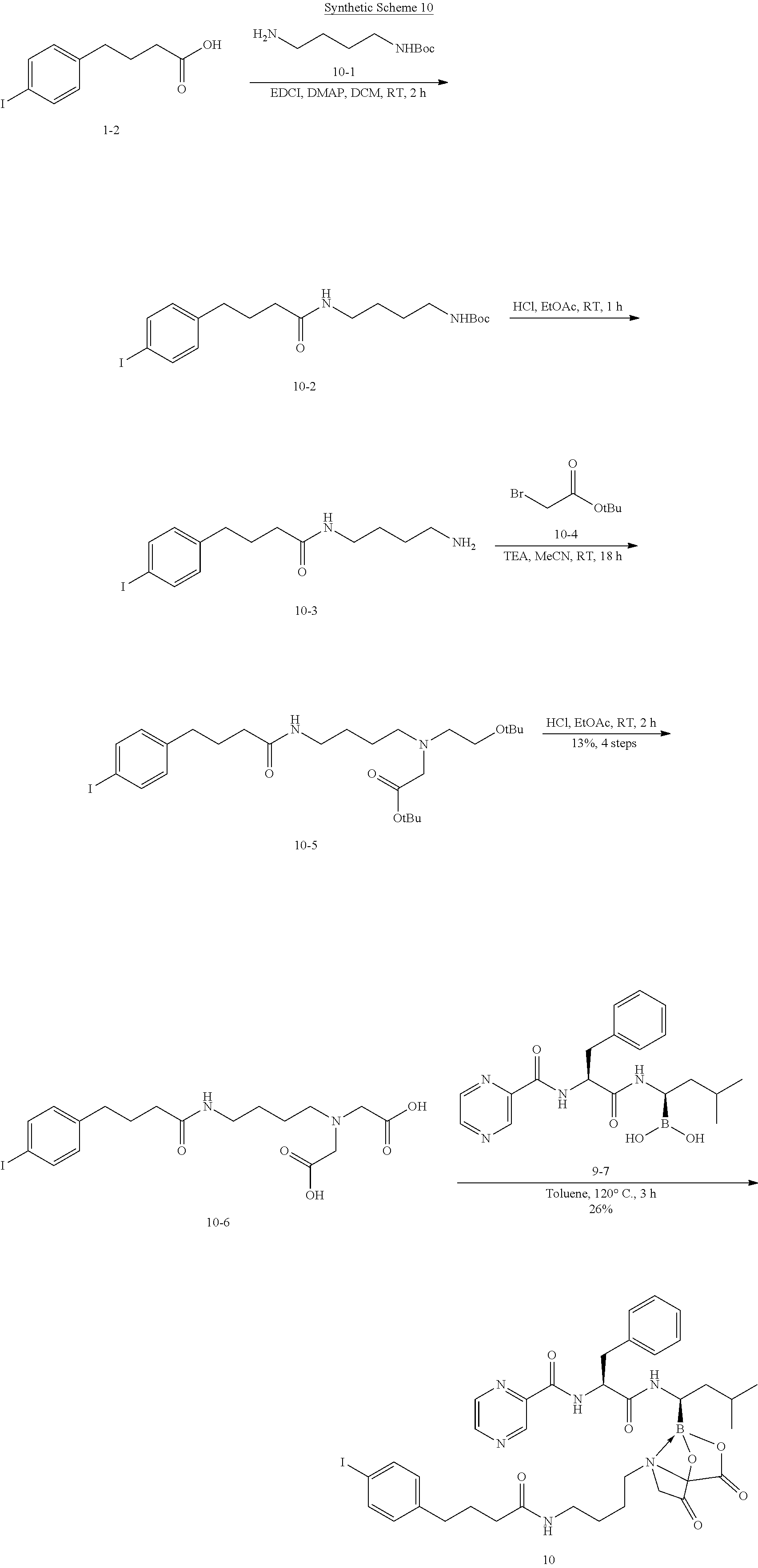

Step 1: A solution of compound 1-2 (400 mg, 1.38 mmol), compound 10-1 (312 mg, 1.66 mmol), DMAP (17 mg, 0.14 mmol) and EDCI (397 mg, 2.07 mmol) in DCM (20 mL) was stirred at room temperature for 2 h. After completion of the reaction, the mixture was diluted with DCM (100 mL) and washed with 1N HCl and brine. The organic layer was dried over anhydrous Na₂SO₄, filtered and concentrated to give the title compound 10-2 (500 mg), which was used at the next step without further purification.

Step 2: The compound 10-2 (500 mg, crude from Step 1) was dissolved in 4N HCl in EtOAc (5 mL), and the solution was stirred for 1 h. After completion of the reaction, the mixture was concentrated to give the title compound 10-3 (450 mg), which was used at the next step without further purification.

Step 3: A mixture of compound 10-3 (450 mg, crude from Step 2), compound 10-4 (600 uL, 4.14 mmol) and triethylamine (1.0 mL, 6.90 mmol) in acetonitrile (10 mL) was stirred at room temperature for 18 h. After completion of the reaction, the reaction mixture was diluted with DCM (100 mL), and washed with water and brine. The organic layer was dried over Na₂SO₄, filtered and concentrated to give the title compound 10-5 (300 mg), which was used at next step without further purification.

Step 4: The compound 10-5 (300 mg, crude from Step 3) was dissolved in 4 N HCl in EtOAc (5 mL) and stirred for 2 h. After completion of the reaction, the mixture was concentrated. The crude product was purified by prep-HPLC (C18 column, eluted with acetonitrile and H₂O, TFA condition) to give the title compound 10-6 (110 mg, yield: 13.4% over 4 steps) as a light yellow oil.

Step 5: A mixture of compound 10-6 (112 mg, 0.235 mmol) and compound 9-7 (90 mg, 0.234 mmol) in toluene (5 mL) was stirred at 120° C. for 3 h. After completion of the reaction, the mixture was concentrated. The residue was washed by MTBE and filtered to give a solid. The crude product was re-dissolved in acetonitrile and purified by pre-TLC (SiO₂, DCM/acetonitrile=3/1) to afford the title compound 10 (51 mg, 26% yield) as a white solid. ¹H NMR (400 MHz, DMSO-d₆) δ 9.07 (s, 1H), 8.89 (d, J=8.3 Hz, 1H), 8.86 (d, J=2.3 Hz, 1H), 8.73 (s, 1H), 7.88-7.79 (m, 1H), 7.59 (dd, J=8.2, 1.9 Hz, 2H), 7.34 (d, J=9.6 Hz, 1H), 7.30-7.20 (m, 4H), 7.19-7.12 (m, 1H), 7.03-6.95 (m, 2H), 4.67 (q, J=7.7 Hz, 1H), 4.17 (d, J=17.3 Hz, 1H), 4.05 (d, J=17.4 Hz, 1H), 3.95 (d, J=16.6 Hz, 1H), 3.63-3.45 (m, 2H), 3.07 (d, J=7.3 Hz, 6H), 2.09-2.03 (m, 2H), 1.77 (p, J=7.4 Hz, 2H), 1.56 (s, 3H), 1.41 (t, J=12.6 Hz, 3H), 1.21 (d, J=17.2 Hz, 2H), 0.85 (d, J=6.6 Hz, 3H), 0.80 (d, J=6.3 Hz, 3H). LCMS: m/z calculated for C₃₇H₄₆BIN₆O₇: 824.52; found: 825.9. [M+H]⁺

### Example 11

### N-((S)-1-(((R)-1-(6-(3-(4-(4-iodophenyl)butanamido)propyl)-4,8-dioxo-1,3,6,2-dioxazaborocan-2-yl)-3-methylbutyl)amino)-1-oxo-3-phenylpropan-2-yl)pyrazine-2-carbox amide (11)

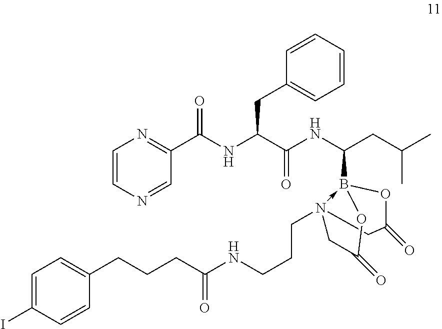 [SMILES: CC(C)C[C@H](NC(=O)[C@H](Cc1ccccc1)NC(=O)c1cnccn1)[B-]12OC(=O)C[N+]1(CCCNC(=O)CCCc1ccc(I)cc1)CC(=O)O2]

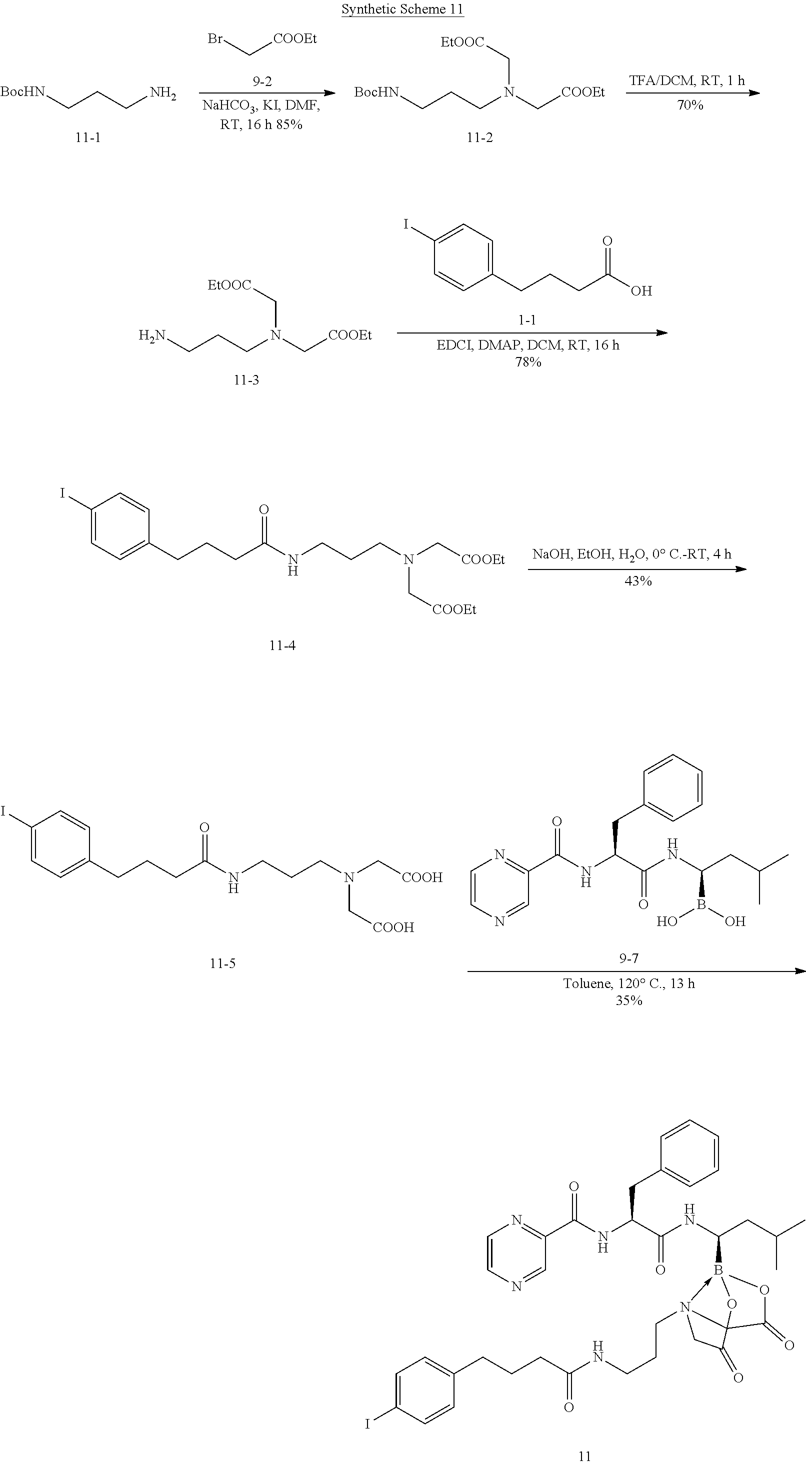

Step 1: A mixture of compound 11-1 (1.0 g, 6.79 mmol), NaHCO₃ (4.8 g, 57.07 mmol), KI (950 mg, 5.72 mmol) and compound 9-2 (9.5 g, 56.89 mmol) in DMF (20 mL) was stirred at room temperature for 16 h. After completion of the reaction, the mixture was partitioned between EtOAc (100 mL) and water (100 mL). The organic layer was washed with brine (100 mL), dried over anhydrous Na₂SO₄ and concentrated. The crude product was purified by silica gel column (eluted with 15% EtOAc in petroleum ether) to afford the title compound 11-2 (1.7 g, yield 85%) as a colorless oil. TLC (DCM:MeOH/10:1): R_{f} (compound 11-1)=0.2; TLC (petroleum ether:EtOAc/2:1): R_{f} (compound 11-2)=0.45.

Step 2: A solution of compound 11-2 (700 mg, 2.02 mmol) and TFA (4 mL) in DCM (4 mL) was stirred at room temperature for 1 h. After completion of the reaction, the mixture was concentrated to give the title compound 11-3 (680 mg, yield 70%) as a colorless oil, which was used at the next step without further purification. TLC (petroleum ether:EtOAc/2:1): R_{f}(compound 11-2)=0.45; TLC (DCM:MeOH/5:1): R_{f} (compound 11-3)=0.15.

Step 3: A mixture of crude compound 11-3 (680 mg, 1.43 mmol), compound 1-1 (527 mg, 1.82 mmol), EDCI (580 mg, 3.04 mmol) and DMAP (25 mg, 0.205 mmol) in DCM (10 mL) was stirred at room temperature for 16 h. After completion of the reaction, the mixture was partitioned between EtOAc (50 mL) and water (20 mL). The organic layer was washed with 1N HCl (50 mL×2), saturated Na₂CO₃ solution, and brine (50 mL), dried over anhydrous Na₂SO₄ and concentrated to afford the title compound 11-4 (580 mg, yield 78%) as a colorless oil, which was used directly at the next step without further purification. LCMS: m/z calculated for C₂₁H₃₁IN₂O₅: 518.39; found: 519.09 [M+H]⁺.

Step 4: A mixture of compound 11-4 (580 mg, 1.12 mmol) and NaOH (290 mg, 7.25 mmol) in EtOH (10 mL) and H₂O (5 mL) was stirred at room temperature for 4 h. After completion of the reaction, the mixture was concentrated to get rid of the organic solvents. The left aqueous solution was adjusted pH=2 and purified by reverse phase flash chromatography (C18 column, eluted with acetonitrile and water, TFA condition) to afford the title compound 11-5 (220 mg, yield 43%) as white solid. LCMS: m/z calculated for C₁₇H₂₃IN₂O₅: 462.28; found: 463.51 [M+H]⁺.

Step 5: A mixture of compound 11-5 (120 mg, 0.259 mmol) and compound 9-7 (83 mg, 0.216 mmol) in toluene (5 mL) was stirred at 120° C. for 13 h. After completion of the reaction, the mixture was concentrated. The residue was purified by pre-TLC (DCM:acetonitrile=3:1) to afford 11 (63 mg, yield 35%) as a white solid. ¹H NMR (400 MHz, DMSO-d₆) δ 9.07 (s, 1H), 8.92-8.83 (m, 2H), 8.77-8.70 (m, 1H), 7.74 (s, 1H), 7.63-7.54 (m, 2H), 7.38 (d, J=9.8 Hz, 1H), 7.27 (dd, J=13.3, 7.8 Hz, 4H), 7.19-7.14 (m, 1H), 6.98 (d, J=7.7 Hz, 2H), 4.67 (d, J=8.4 Hz, 1H), 4.17-4.05 (m, 2H), 3.98 (d, J=16.6 Hz, 1H), 3.53 (d, J=22.0 Hz, 2H), 3.07 (d, J=11.9 Hz, 6H), 2.08 (d, J=6.9 Hz, 2H), 1.80-1.70 (m, 3H), 1.57 (s, 2H), 1.42 (t, J=13.1 Hz, 2H), 1.21 (d, J=18.3 Hz, 2H), 0.82 (dd, J=22.8, 6.3 Hz, 6H). LCMS: m/z calculated for C₃₆H₄₄BIN₆O₇: 810.50; found: 811.63 [M+H]⁺.

### Example 12

### N-((S)-1-(((R)-1-(6-(5-(4-(4-iodophenyl)butanamido)pentyl)-4,8-dioxo-1,3,6,2-dioxazaborocan-2-yl)-3-methylbutyl)amino)-1-oxo-3-phenylpropan-2-yl)pyrazine-2-carbox amide (12)

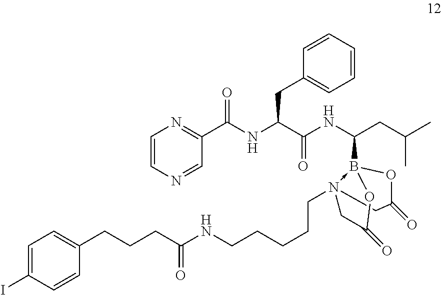 [SMILES: CC(C)C[C@H](NC(=O)[C@H](Cc1ccccc1)NC(=O)c1cnccn1)[B-]12OC(=O)C[N+]1(CCCCCNC(=O)CCCc1ccc(I)cc1)CC(=O)O2]

### Synthetic Scheme 12

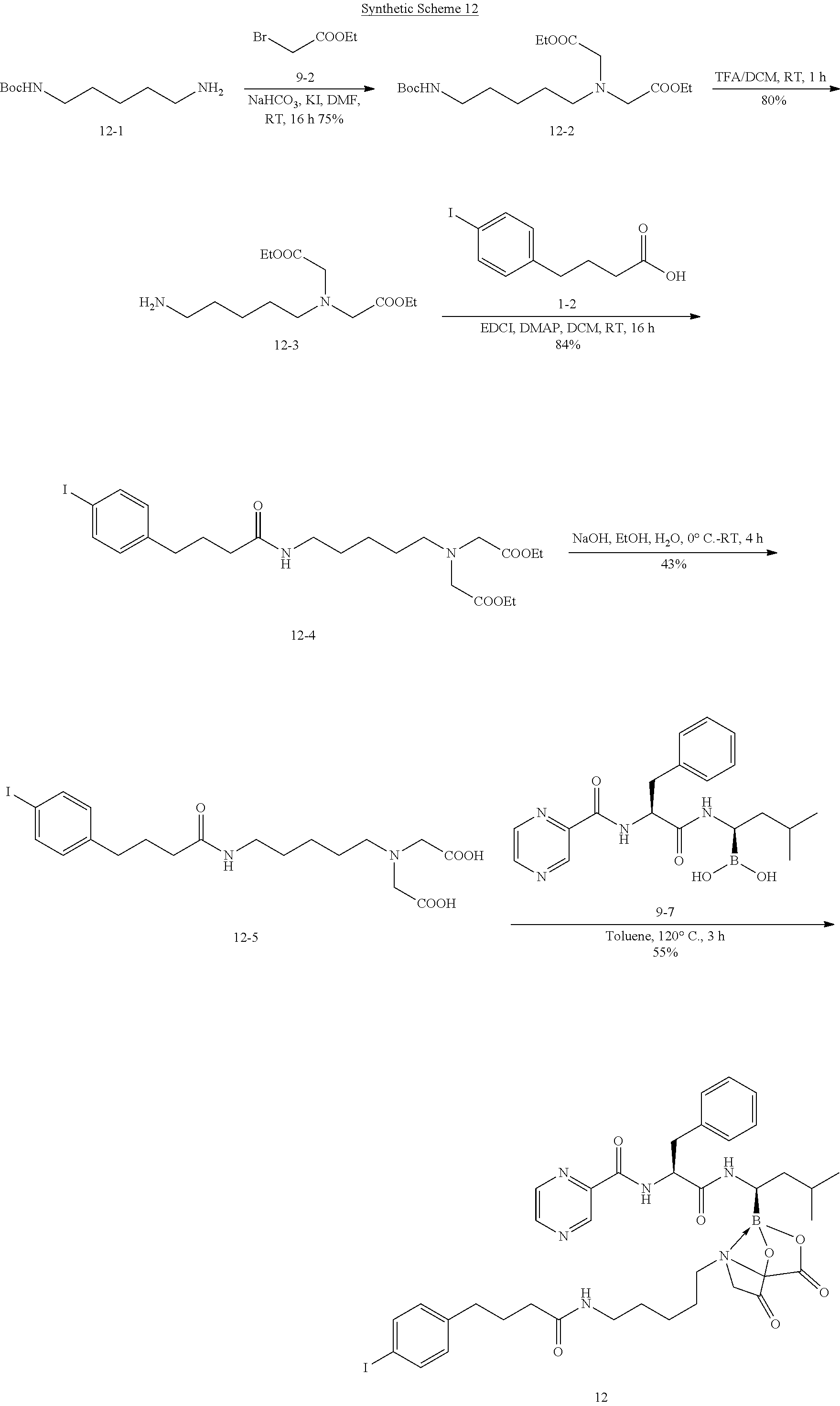

Step 1: A mixture of compound 12-1 (500 mg, 2.47 mmol), NaHCO₃ (2.1 g, 24.9 mmol), KI (410 mg, 2.47 mmol) and compound 9-2 (4.1 g, 24.5 mmol) in DMF (10 mL) was stirred at room temperature for 16 h. After completion of the reaction, the mixture was partitioned between EtOAc (50 mL) and water (50 mL). The organic layer was washed with brine (50 mL), dried over anhydrous Na₂SO₄ and concentrated. The crude product was purified by silica gel column (eluted with 15% EtOAc in petroleum ether) to afford the title compound 12-2 (700 mg, yield 75%) as colorless oil. TLC (DCM:MeOH/10:1): R_{f} (compound 12-1)=0.2; TLC (petroleum ether:EtOAc/2:1): R_{f} (compound 12-2)=0.55.

Step 2: A solution of compound 12-2 (700 mg, 1.87 mmol) and TFA (4 mL) in DCM (4 mL) was stirred at room temperature for 1 h. After completion of the reaction, the mixture was concentrated to give the title compound 12-3 (760 mg, yield 80%) as a colorless oil, which was used at the next step without further purification. TLC (petroleum ether:EtOAc/2:1): R_{f}(compound 12-2)=0.55; TLC (DCM:MeOH/5:1): R_{f} (compound 12-3)=0.18.

Step 3: A mixture of crude compound 12-3 (660 mg, 1.51 mmol), compound 1-2 (488 mg, 1.68 mmol), EDCI (540 mg, 2.82 mmol) and DMAP (23 mg, 0.189 mmol) in DCM (10 mL) was stirred at room temperature for 16 h. After completion of the reaction, the mixture was partitioned between EtOAc (50 mL) and water (20 mL). The organic layer was washed with 1N HCl (50 mL×2), saturated Na₂CO₃ solution and brine (50 mL), dried over anhydrous Na₂SO₄ and concentrated to afford the title compound 12-4 (700 mg, yield 84%) as a colorless oil, which was used at the next step without further purification. TLC (petroleum ether:EtOAc/2:1): R_{f}(compound 12-3)=0.5; R_{f} (compound 12-4)=0.2.

Step 4: A mixture of compound 12-4 (700 mg, 1.28 mmol) and NaOH (270 mg, 6.75 mmol) in EtOH (10 mL) and H₂O (5 mL) was stirred at room temperature for 4 h. After completion of the reaction, the mixture was concentrated to get rid of the organic solvents. The left aqueous solution was adjusted to pH=2 and purified directly by reverse phase flash chromatography (C18 column, eluted with acetonitrile and water, TFA condition) to afford the title compound 12-5 (270 mg, yield 43%) as white solid. LCMS: m/z calculated for C₁₉H₂₇IN₂O₅: 490.34; found: 491.30 [M+H]⁺.

Step 5: A mixture of compound 12-5 (153 mg, 0.312 mmol) and compound 9-7 (100 mg, 0.260 mmol) in toluene (5 mL) was stirred at 120° C. for 13 h. After completion of the reaction, the mixture was concentrated. The residue was purified by pre-TLC (DCM:acetonitrile=3:1) to afford 12 (67 mg, yield 25%) as a white solid. ¹H NMR (400 MHz, DMSO-d₆) δ 9.05 (d, J=3.0 Hz, 1H), 8.94-8.82 (m, 2H), 8.71 (d, J=3.3 Hz, 1H), 7.81 (s, 1H), 7.56 (dd, J=8.1, 2.8 Hz, 2H), 7.35 (d, J=10.1 Hz, 2H), 7.30-7.21 (m, 4H), 7.19-7.13 (m, 1H), 6.96 (dd, J=8.4, 2.8 Hz, 2H), 4.66 (s, 1H), 4.15 (dd, J=17.5, 3.0 Hz, 1H), 4.07-3.99 (m, 1H), 3.93 (dd, J=16.8, 3.0 Hz, 1H), 3.05 (d, J=8.4 Hz, 6H), 2.03 (d, J=8.1 Hz, 2H), 1.75 (d, J=8.0 Hz, 2H), 1.54 (s, 4H), 1.41 (d, J=9.9 Hz, 3H), 1.15 (s, 4H), 0.83 (dd, J=6.6, 3.1 Hz, 3H), 0.79 (dd, J=6.6, 3.1 Hz, 3H). LCMS: m/z calculated for C₃₈H₄₈BIN₆O₇: 838.55; found: 839.50 [M+H]⁺.

### Example 13

### N-((2S)-1-(((1R)-1-(6-(4-(2-(4-isobutylphenyl)propanamido)butyl)-4,8-dioxo-1,3,6,2-dioxazaborocan-2-yl)-3-methylbutyl)amino)-1-oxo-3-phenylpropan-2-yl)pyrazine-2-carboxamide (13)

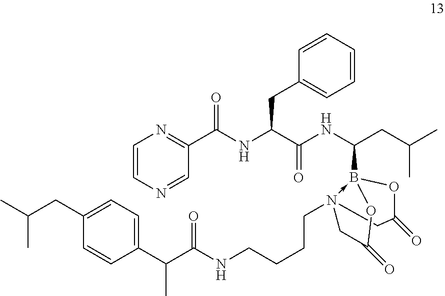 [SMILES: CC(C)Cc1ccc(C(C)C(=O)NCCCC[N+]23CC(=O)O[B-]2([C@H](CC(C)C)NC(=O)[C@H](Cc2ccccc2)NC(=O)c2cnccn2)OC(=O)C3)cc1]

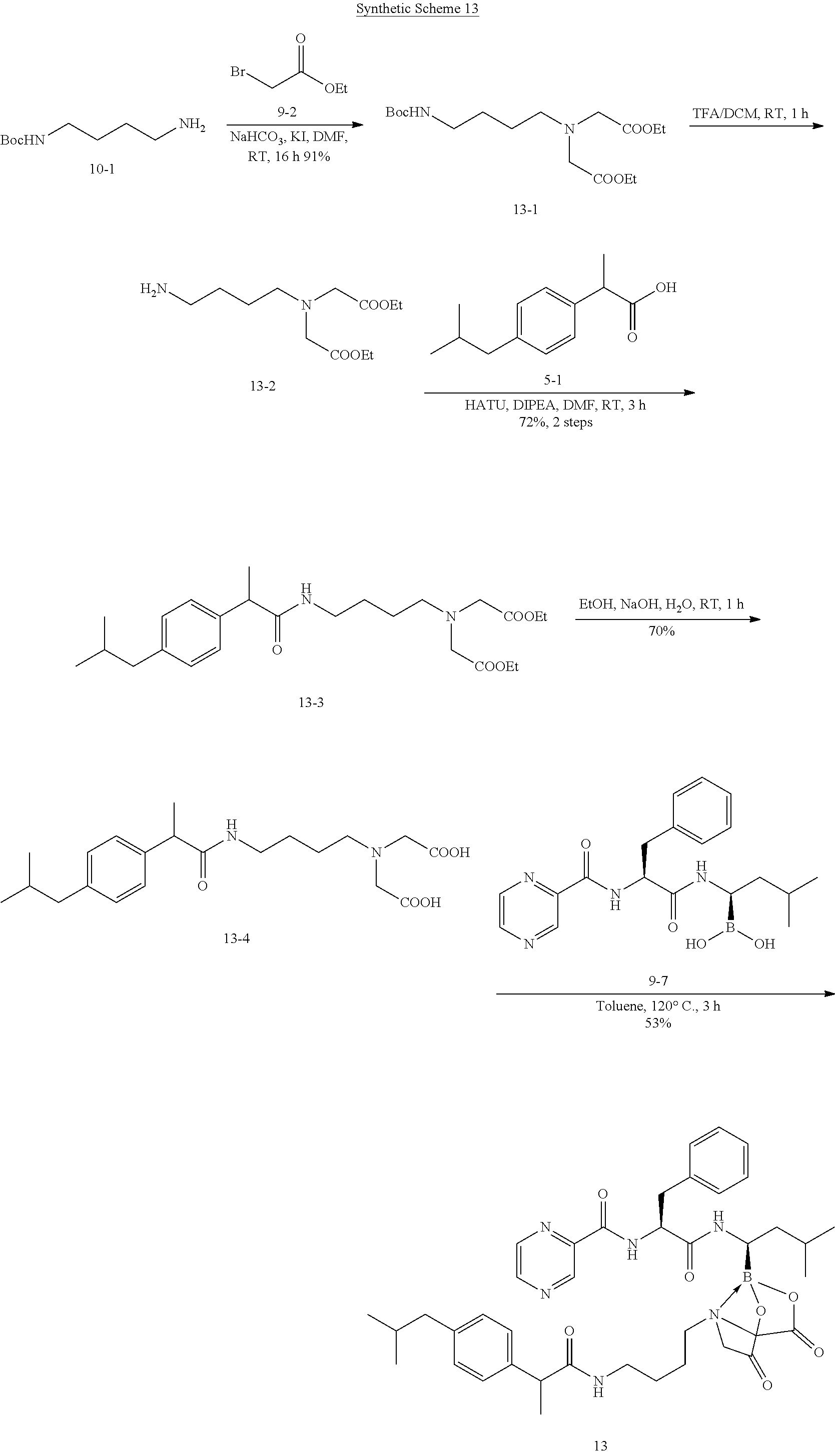

Step 1: A mixture of compound 10-1 (2 g, 10.62 mmol), compound 9-2 (17.7 g, 106.23 mmol), NaHCO₃ (8.92 g, 106.23 mmol) and KI (1.76 g, 10.62 mmol) in DMF (20 mL) was stirred at room temperature for 16 h. The reaction mixture was diluted with EtOAc (100 mL) and washed with water (100 mL). The organic layer was washed with brine, dried over anhydrous Na₂SO₄, filtered and concentrated. The crude product was purified by silica gel column (eluted with 30% EtOAc in Petroleum ether) to afford the title compound 13-1 (3.5 g, yield 91%) as a yellow oil.

Step 2: To a solution of compound 13-1 (2 g, 5.55 mmol) in DCM (16 mL) was added TFA (4 mL) at room temperature and the mixture stirred for 1 h. After completion of the reaction, the mixture was concentrated to afford the crude compound 13-2 (3 g), which was used directly at the next step.

Step 3: A mixture of compound 5-1 (1.14 g, 5.53 mmol), HATU (3.15 g, 8.29 mmol) and DIPEA (1.37 mL, 8.29 mmol) in DMF (2 mL) was stirred at room temperature for 10 minutes. Compound 13-2 (3 g, crude from Step 2) was added and the mixture was stirred for 3 h. After completion of the reaction, the mixture was diluted with EtOAc (50 mL) and washed with water (50 mL). The organic layer was washed with brine, dried over anhydrous Na₂SO₄, filtered and concentrated. The crude product was purified by reverse phase flash chromatography (C18 column, eluted with acetonitrile and H₂O, HCl condition) to afford the title compound 13-3 (1.8 g, yield 72%, 2 steps). LCMS: m/z calculated for C₂₅H₄₀N₂O₅: 448.29; found: 473.32 [M+H]⁺.

Step 4: To a mixture of compound 13-3 (1.8 g, 4.01 mmol) in EtOH (8 mL) and H₂O (4 mL) was added LiOH (336.7 mg, 20.06 mmol). The mixture was stirred at room temperature for 1 h. After completion of the reaction, the mixture was adjusted to pH=3 with 1 N HCl and concentrated. The crude product was purified by reverse phase flash chromatography (C18 column, eluted with acetonitrile and H₂O, HCl condition) to afford the title compound 13-4 (1.1 g, yield 70%). LCMS: m/z calculated for C₂₁H₃₂N₂O₅: 392.50; found: 393.33 [M+H]⁺.

Step 5: A mixture of compound 13-4 (1 g, 0.36 mmol) and compound 9-7 (115 mg, 0.30 mmol) in toluene (15 mL) was stirred at 120° C. for 17 h. After completion of the reaction, the mixture was concentrated. The crude product was purified by silica gel column (eluted with 30% acetonitrile in DCM) to afford the title compound 13 (1 g, yield 53%) as a pale-yellow solid. ¹H NMR (500 MHz, DMSO-d₆) δ 9.09 (dd, J=3.8, 1.5 Hz, 1H), 8.92-8.85 (m, 2H), 8.73 (ddd, J=5.1, 2.5, 1.5 Hz, 1H), 7.97 (td, J=5.8, 2.1 Hz, 1H), 7.35 (dd, J=9.9, 6.0 Hz, 1H), 7.31-7.15 (m, 7H), 7.06 (d, J=7.9 Hz, 2H), 4.73-4.63 (m, 1H), 4.12 (dd, J=17.4, 3.6 Hz, 1H), 4.03 (dd, J=17.3, 1.7 Hz, 1H), 3.98-3.89 (m, 1H), 3.56 (qd, J=8.1, 7.0, 3.5 Hz, 2H), 3.48 (dd, J=16.7, 8.7 Hz, 1H), 3.15-2.97 (m, 6H), 2.38 (d, J=7.2 Hz, 2H), 1.78 (dp, J=13.5, 6.7 Hz, 1H), 1.62-1.50 (m, 2H), 1.50-1.40 (m, 2H), 1.40-1.33 (m, 2H), 1.32 (dd, J=7.0, 1.4 Hz, 3H), 1.21 (ddt, J=13.7, 10.2, 4.5 Hz, 1H), 0.92-0.74 (m, 12H). LCMS: m/z calculated for C₄₀H₅₃BN₆O₇: 740.41; found: 742.01 [M+H]⁺.

### Example 14

### N-((S)-1-(((R)-1-(6-(4-((S)-2-(6-methoxynaphthalen-2-yl)propanamido)butyl)-4,8-dioxo-1,3,6,2-dioxazaborocan-2-yl)-3-methylbutyl)amino)-1-oxo-3-phenylpropan-2-yl) pyrazine-2-carboxamide (14)

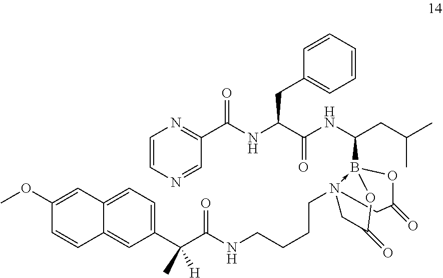 [SMILES: COc1ccc2cc([C@H](C)C(=O)NCCCC[N+]34CC(=O)O[B-]3([C@H](CC(C)C)NC(=O)[C@H](Cc3ccccc3)NC(=O)c3cnccn3)OC(=O)C4)ccc2c1]

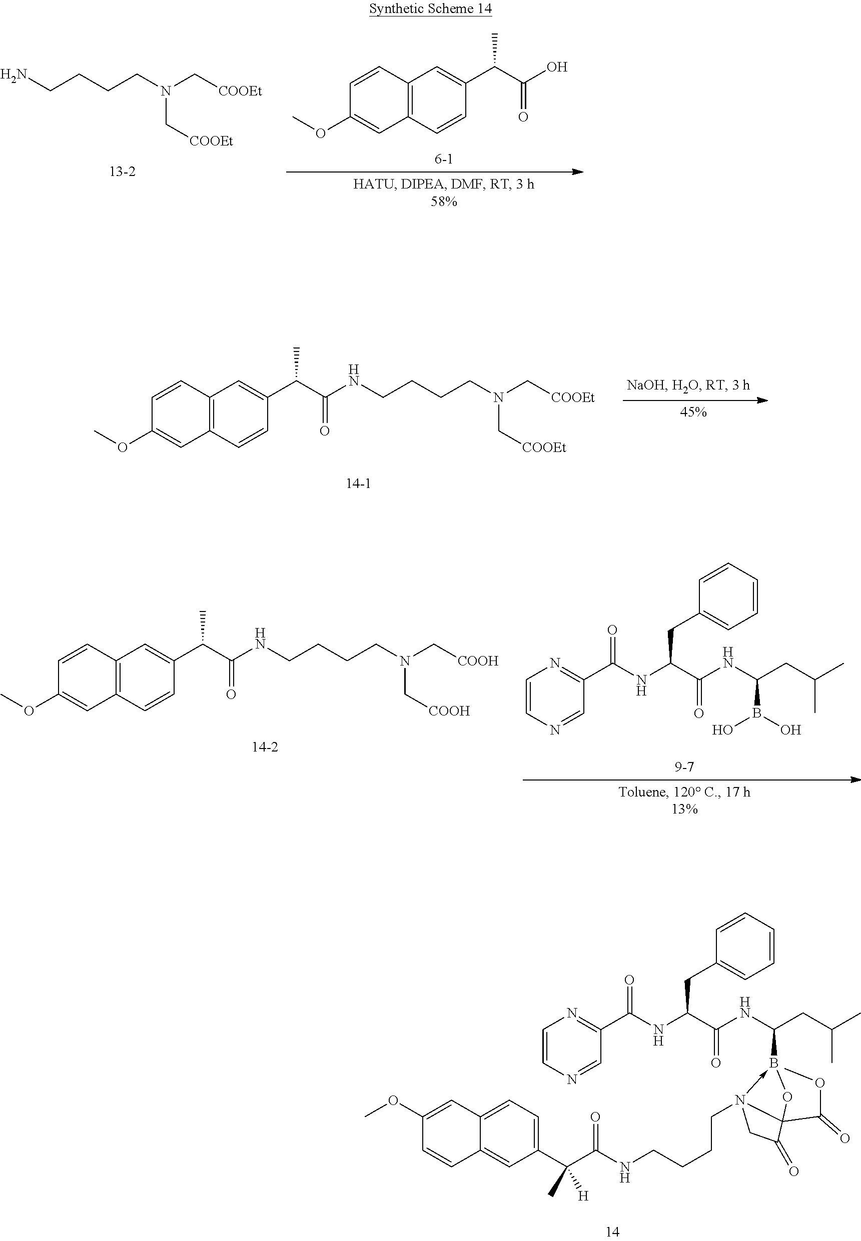

Step 1: A mixture of compound 6-1 (254 mg, 1.11 mmol), DIPEA (445 mg, 3.45 mmol), compound 13-2 (415 mg, crude, ˜70% purity, 1.12 mmol) and HATU (787 mg, 2.07 mmol) in DMF (3 mL) was stirred at room temperature for 3 h. After completion of the reaction, the mixture was partitioned between EtOAc (50 mL) and water (50 mL). The organic layer was washed with brine, dried over anhydrous Na₂SO₄ and concentrated. The crude product was purified by reverse phase flash chromatography (C18 column, eluted with acetonitrile and water, HCl condition). The desired component was lyophilized to afford the title compound 14-1 (380 mg, yield 58%). LCMS: m/z calculated for C₂₆H₃₆N₂O₆: 472.3; found: 473.32 [M+H]⁺.

Step 2: A mixture of compound 14-1 (380 mg, 0.80 mmol) and NaOH (160 mg, 4.01 mmol) in EtOH (5 mL) and H₂O (5 mL) was stirred at room temperature for 3 h. After completion of the reaction, the mixture was adjusted to pH=7 with 1 N HCl. The resulting mixture was concentrated and purified by reverse phase flash chromatography (C18 column, eluted with acetonitrile and water, HCl condition). The desired component was lyophilized to afford the title compound 14-2 (150 mg, yield 45%). LCMS: m/z calculated for C₂₆H₃₆N₂O₆: 416.19; found: 417.13. [M+H]⁺

Step 3: A mixture of compound 14-2 (150 mg, 0.36 mmol) and compound 9-7 (115 mg, 0.30 mmol) in toluene (3 mL) was stirred at 120° C. for 17 h. After completion of the reaction, the mixture was concentrated to get rid of organic solvents. The crude product was purified by pre-HPLC (C18 column, eluted with acetonitrile and H₂O, neutral condition) to afford the title compound 14 (30 mg, yield 13%). ¹H NMR (400 MHz, DMSO-d₆) δ 9.05 (d, J=4.0 Hz, 1H), 8.86 (d, J=13.7 Hz, 2H), 8.68 (s, 1H), 8.05 (d, J=6.4 Hz, 1H), 7.72 (dt, J=13.6, 6.0 Hz, 3H), 7.47-7.40 (m, 1H), 7.34 (d, J=9.1 Hz, 1H), 7.28-7.19 (m, 5H), 7.18-7.08 (m, 2H), 4.66 (s, 1H), 4.09 (d, J=17.7 Hz, 1H), 3.98 (d, J=17.5 Hz, 1H), 3.88 (d, J=14.9 Hz, 1H), 3.83 (d, J=4.3 Hz, 3H), 3.71 (s, 2H), 3.06 (s, 6H), 1.53 (s, 2H), 1.39 (q, J=13.4, 9.6 Hz, 6H), 1.28-1.12 (m, 4H), 0.83 (t, J=5.2 Hz, 3H), 0.79 (t, J=5.5 Hz, 3H). LCMS: m/z calculated for C₄₁H₄₉BN₆O₈: 764.37; found: 765.43. [M+H]⁺.

### Example 15

### N-((S)-1-(((R)-1-(6-(4-((R)-2-(4-isobutylphenyl)propanamido)butyl)-4,8-dioxo-1,3,6,2-dioxazaborocan-2-yl)-3-methylbutyl)amino)-1-oxo-3-phenylpropan-2-yl)pyrazine-2-carboxamide (15)

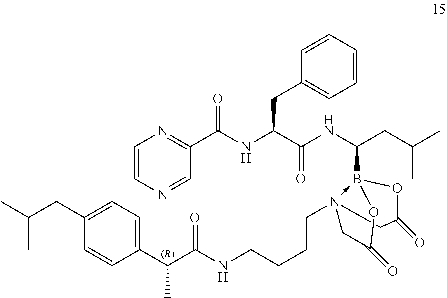

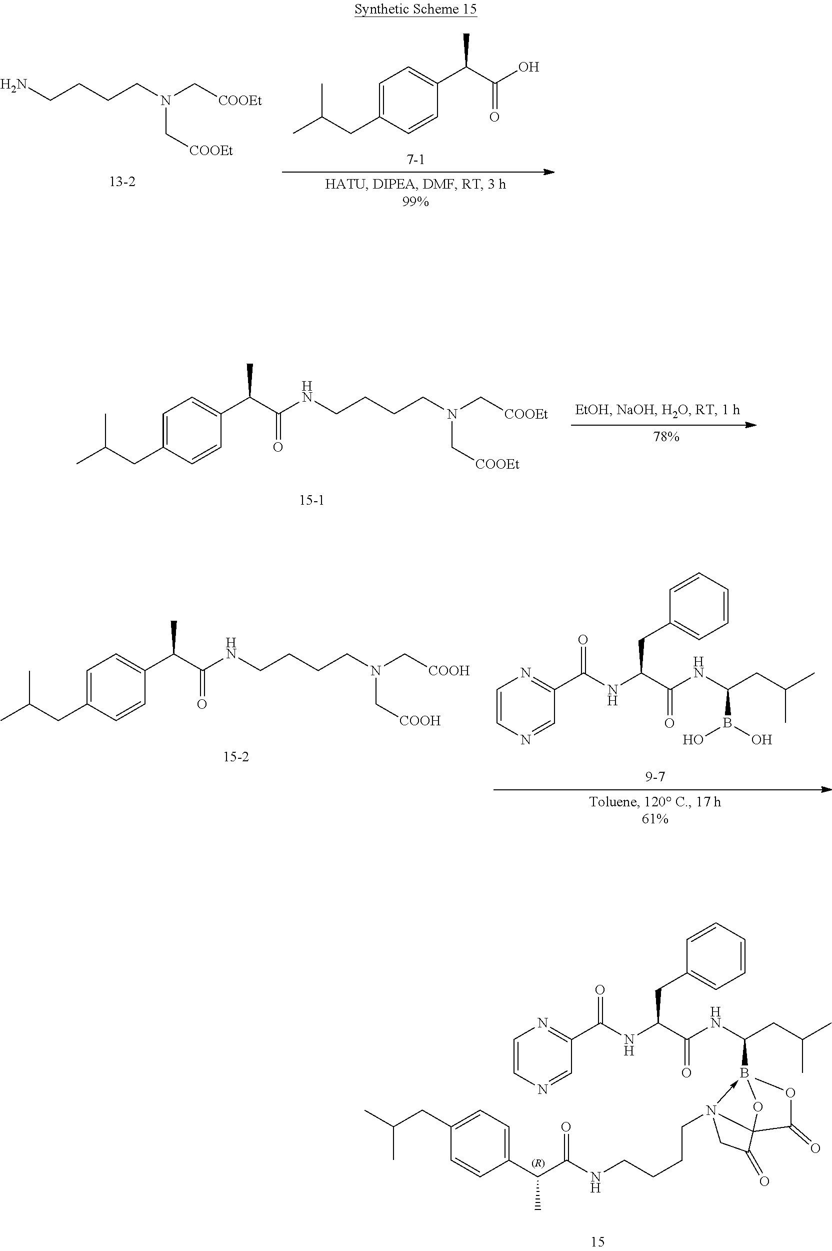

Step 1: A mixture of compound 7-1 (143 mg, 0.69 mmol), HATU (396 mg, 1.04 mmol) and DIPEA (224 mg, 1.73 mmol) in DMF (2 mL) was stirred at room temperature for 10 minutes, compound 13-2 (300 mg, crude, ˜70% purity, 0.81 mmol) was added. After stirring at room temperature for 3 h, the mixture was diluted with EtOAc (20 mL) and washed with water (20 mL). The organic layer was washed with brine, dried over anhydrous Na₂SO₄, filtered and concentrated. The crude product was purified by reverse phase flash chromatography (C18 column, eluted with acetonitrile and H₂O, HCl condition) to afford the title compound 15-1 (307 mg, yield 99%). LCMS: m/z calculated for C₂₅H₄₀N₂O₅: 448.60; found: 449.62 [M+H]⁺.

Step 2: To a mixture of compound 15-1 (337 mg, 0.75 mmol) in EtOH (2 mL) and H₂O (2 mL) was added LiOH·H₂O (157.6 mg, 3.76 mmol). Then the mixture was stirred at room temperature for 1 h. After completion of the reaction, the mixture was adjusted to pH=7 with 1 N HCl and concentrated. The crude product was purified by reverse phase flash chromatography (C18 column, eluted with acetonitrile and H₂O, HCl condition) to afford the title compound 15-2 (231 mg, yield 78%). LCMS: m/z calculated for C₂₅H₄₀N₂O₅: 392.23; found: 393.33 [M+H]⁺.

Step 3: A mixture of compound 15-2 (100 mg, 0.25 mmol) and compound 9-7 (81.6 mg, 0.21 mmol) in toluene (5 mL) was stirred at 120° C. for 17 h. After completion of the reaction, the mixture was concentrated. The crude product was purified by pre-TLC (SiO₂, acetonitrile/DCM=1/3) to afford the title compound 15 (97 mg, yield 61%) as a white solid. ¹H NMR (500 MHz, DMSO-d₆) δ 9.09 (d, J=1.5 Hz, 1H), 8.92-8.86 (m, 2H), 8.74 (dd, J=2.5, 1.5 Hz, 1H), 7.97 (t, J=5.8 Hz, 1H), 7.34 (d, J=9.8 Hz, 1H), 7.31-7.27 (m, 2H), 7.27-7.23 (m, 2H), 7.22-7.19 (m, 2H), 7.19-7.15 (m, 1H), 7.08-7.03 (m, 2H), 4.68 (q, J=7.7 Hz, 1H), 4.11 (d, J=17.4 Hz, 1H), 4.03 (d, J=17.3 Hz, 1H), 3.92 (d, J=16.7 Hz, 1H), 3.56 (qd, J=6.9, 3.1 Hz, 2H), 3.49 (d, J=16.8 Hz, 1H), 3.13-2.98 (m, 6H), 2.38 (d, J=7.1 Hz, 2H), 1.78 (hept, J=6.8 Hz, 1H), 1.57 (ddt, J=13.7, 6.7, 3.6 Hz, 2H), 1.49-1.34 (m, 4H), 1.32 (d, J=7.1 Hz, 3H), 1.20 (ddd, J=17.2, 10.2, 5.0 Hz, 1H), 0.83 (dt, J=13.6, 6.9 Hz, 12H). LCMS: m/z calculated for C₄₀H₅₃BN₆O₇: 740.41; found: 741.94 [M+H]⁺.

### Example 16

### N-((S)-1-(((R)-1-(6-(4-((S)-2-(4-isobutylphenyl)propanamido)butyl)-4,8-dioxo-1,3,6,2-dioxazaborocan-2-yl)-3-methylbutyl)amino)-1-oxo-3-phenylpropan-2-yl)pyrazine-2-carboxamide (16)

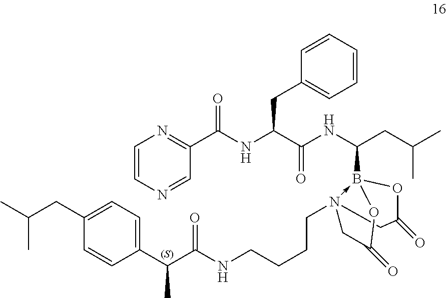

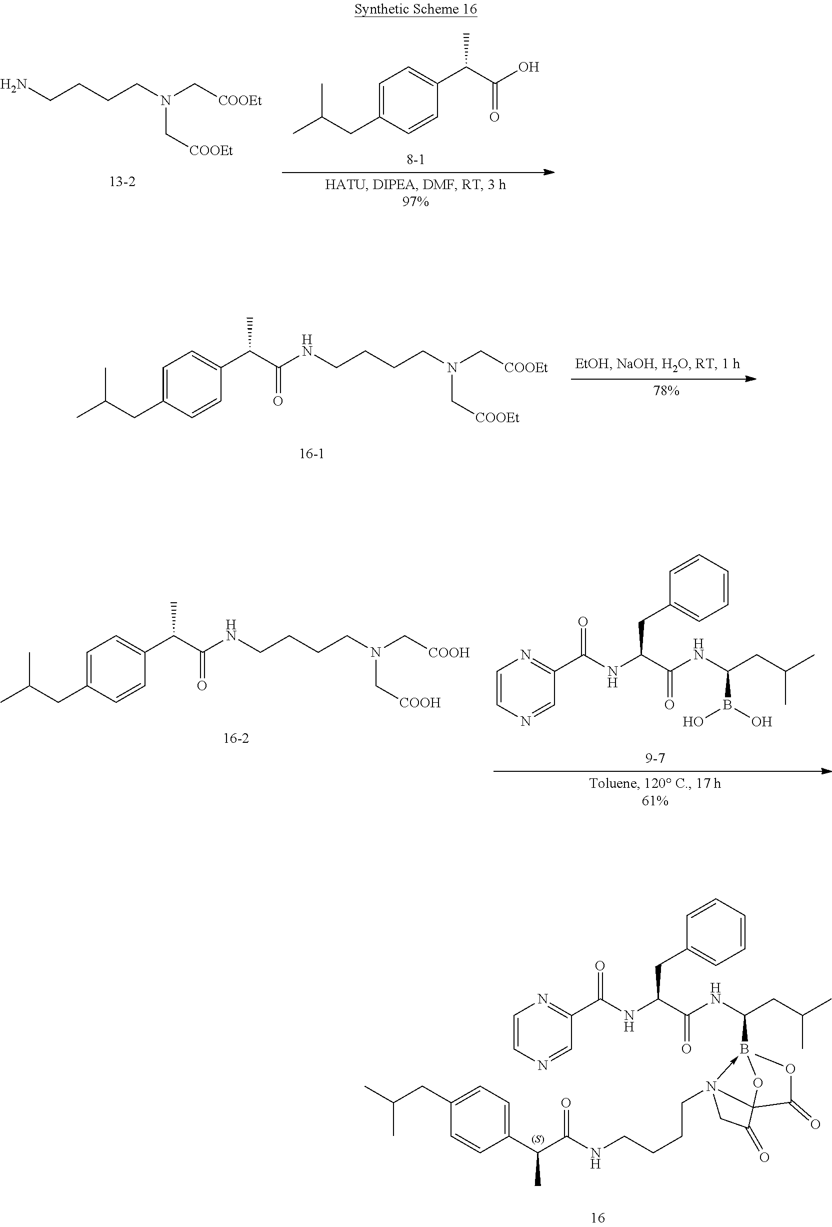

Step 1: A mixture of compound 8-1 (334 mg, 1.62 mmol), HATU (924 mg, 2.42 mmol) and DIPEA (716.8 μL, 4.04 mmol) in DMF (5 mL) was stirred at room temperature for 10 minutes, compound 13-2 (800 mg, crude, 70% purity, 2.15 mmol) was added. After stirring at room temperature for 3 h, the mixture was diluted with EtOAc (50 mL) and washed with water (50 mL). The organic layer was washed with brine, dried over anhydrous Na₂SO₄, filtered and concentrated. The crude product was purified by reverse phase flash chromatography (C18 column, eluted with acetonitrile and H₂O, HCl condition) to afford the title compound 16-1 (706 mg, yield 97%). LCMS: m/z calculated for C₂₅H₄₀N₂O₅: 448.60; found: 449.62 [M+H]⁺.

Step 2: To a mixture of compound 16-1 (806 mg, 1.80 mmol) in EtOH (5 mL) and H₂O (5 mL) was added LiOH·H₂O (376.9 mg, 8.98 mmol), and the mixture was stirred at room temperature for 1 h. After completion of the reaction, the mixture was adjusted to pH=7 with 1 N HCl and concentrated. The crude product was purified by reverse phase flash chromatography (C18 column, eluted with acetonitrile and H₂O, HCl condition) to afford the title compound 16-2 (549 mg, yield 78%). LCMS: m/z calculated for C₂₅H₄₀N₂O₅: 392.23; found: 393.33 [M+H]⁺.

Step 3: A mixture of compound 16-2 (100 mg, 0.25 mmol) and compound 9-7 (81.6 mg, 0.21 mmol) in toluene (5 mL) was stirred at 120° C. for 17 h. After completion of the reaction, the mixture was concentrated. The crude product was purified by pre-TLC (Acetonitrile/DCM=1/3) to afford the compound 16 (98 mg, yield 62%) as a white solid. ¹H NMR (500 MHz, DMSO-d₆) δ 9.09 (d, J=1.5 Hz, 1H), 8.90-8.85 (m, 2H), 8.74-8.71 (m, 1H), 7.97 (t, J=5.8 Hz, 1H), 7.36 (d, J=9.8 Hz, 1H), 7.31-7.27 (m, 2H), 7.27-7.23 (m, 2H), 7.23-7.20 (m, 2H), 7.19-7.15 (m, 1H), 7.08-7.04 (m, 2H), 4.75-4.64 (m, 1H), 4.12 (d, J=17.4 Hz, 1H), 4.03 (d, J=17.3 Hz, 1H), 3.92 (d, J=16.7 Hz, 1H), 3.61-3.52 (m, 2H), 3.47 (d, J=16.7 Hz, 1H), 3.14-2.98 (m, 6H), 2.38 (d, J=7.1 Hz, 2H), 1.79 (dh, J=13.6, 6.8 Hz, 1H), 1.62-1.50 (m, 2H), 1.50-1.34 (m, 4H), 1.32 (d, J=7.0 Hz, 3H), 1.21 (ddd, J=17.2, 9.5, 4.3 Hz, 1H), 0.84 (td, J=10.4, 9.7, 6.5 Hz, 12H). LCMS: m/z calculated for C₄₀H₅₃BN₆O₇: 740.41; found: 741.73 [M+H]⁺.

### Example 17

### N-((S)-1-(((R)-1-(6-(5-((S)-2-(4-isobutylphenyl)propanamido)pentyl)-4,8-dioxo-1,3,6,2-dioxa zaborocan-2-yl)-3-methylbutyl)amino)-1-oxo-3-phenylpropan-2-yl)pyrazine-2-carboxamide (17)

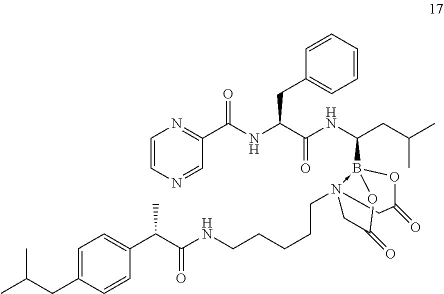 [SMILES: CC(C)Cc1ccc([C@H](C)C(=O)NCCCCC[N+]23CC(=O)O[B-]2([C@H](CC(C)C)NC(=O)[C@H](Cc2ccccc2)NC(=O)c2cnccn2)OC(=O)C3)cc1]

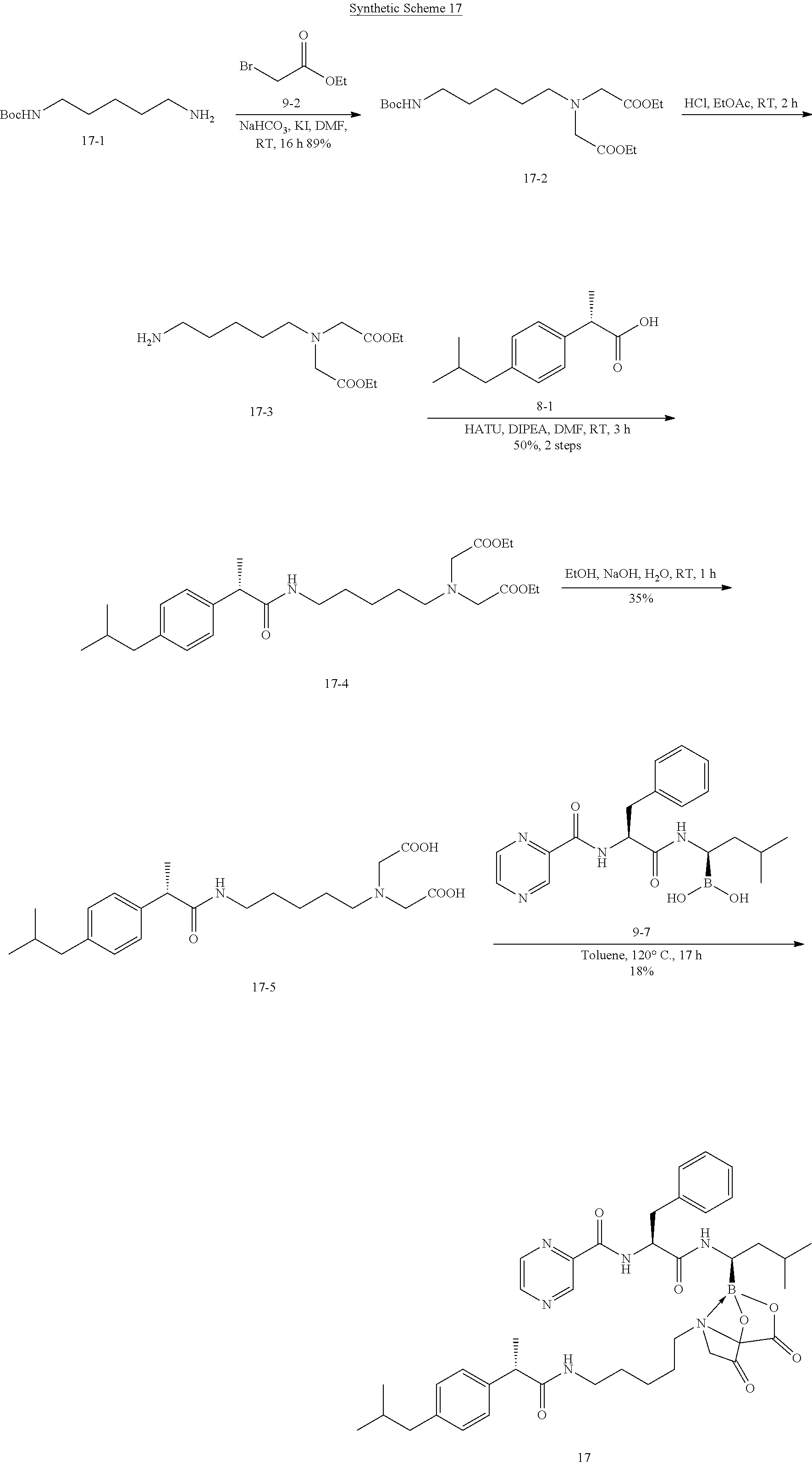

Step 1: To a solution of compound 17-1 (1.0 g, 4.94 mmol) in DMF (10 mL) was added NaHCO₃ (4.15 g, 49.39 mmol), KI (820 mg, 4.93 mmol) and compound 9-2 (4.13 g, 24.73 mmol). The mixture was stirred at room temperature for 16 h. After completion of the reaction, the mixture was partitioned between EtOAc (100 mL) and water (100 mL). The organic layer was washed with brine, dried over anhydrous Na₂SO₄ and concentrated. The residue was purified by silica gel column (eluted with 30% EtOAc in petroleum ether) to afford the title compound 17-2 (1.66 g, yield 89%).

Step 2: A mixture of compound 17-2 (1 g, 2.67 mmol) in 4 N HCl (EtOAc solution, 10 mL) and MeOH (0.5 mL) was stirred at room temperature for 2 h. After completion of the reaction, the mixture was concentrated to afford the title compound 17-3 (1 g, crude), which was used at the next step without further purification.

Step 3: A solution of compound 17-3 (200 mg, 0.673 mmol), compound 8-1 (660 mg, 3.20 mmol), DIPEA (862 mg, 6.68 mmol) and HATU (1.52 g, 3.99 mmol) in DMF (5 mL) was stirred at room temperature for 3 h. After completion of the reaction, the mixture was diluted with EtOAc (50 mL) and washed with water (50 mL×2) and brine (50 mL). The organic layer was dried by anhydrous Na₂SO₄, filtered and concentrated. The crude product was purified by silica gel column (eluted with 30% EtOAc in petroleum ether) to afford the title compound 17-4 (623 mg, yield 50% over 2 steps). LCMS: m/z calculated for C₂₆H₄₂N₂O₅: 462.63; found: 463.95 [M+H]⁺.

Step 4: To a solution of compound 17-4 (620 mg, 1.34 mmol) in EtOH (6 mL) and H₂O (3 mL) was added NaOH (214 mg, 5.36 mmol) at 0° C. and the reaction was stirred at room temperature for 1 h. The mixture was concentrated and adjusted to pH=2 with 1 N HCl at 0° C. The aqueous mixture was purified by reverse phase flash chromatography (C18 column, eluted with 55% acetonitrile in water, HCl condition). The desired components were lyophilized to afford the title compound 17-5 (209 mg, yield 35%) as a white solid. LCMS: m/z calculated for C₂₂H₃₄N₂O₅: 406.52; found: 407.83 [M+H]⁺.

Step 5: A mixture of compound 17-5 (172 mg, 0.388 mmol) and compound 9-7 (149 mg, 0.388 mmol) in toluene (3 mL) was stirred at 120° C. for 17 h. After completion of the reaction, the mixture was concentrated. The crude product was purified by pre-TLC (acetonitrile:dichloromethane=1:3) to afford 17 (55 mg, yield 18%) as a white solid. LCMS: m/z calculated for C₄₁H₅₅BN₆O₇: 754.74; found: 755.93 [M+H]⁺.

### Example 18

### N-((S)-1-(((R)-1-(6-(6-((S)-2-(4-isobutylphenyl)propanamido)hexyl)-4,8-dioxo-1,3,6,2-dioxaz aborocan-2-yl)-3-methylbutyl)amino)-1-oxo-3-phenylpropan-2-yl)pyrazine-2-Carboxamide (18)

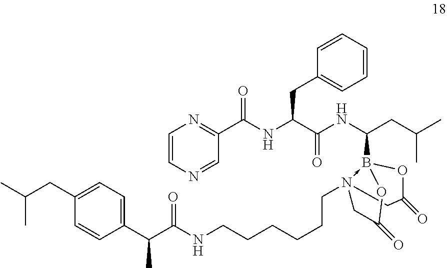 [SMILES: CC(C)Cc1ccc([C@H](C)C(=O)NCCCCCC[N+]23CC(=O)O[B-]2([C@H](CC(C)C)NC(=O)[C@H](Cc2ccccc2)NC(=O)c2cnccn2)OC(=O)C3)cc1]

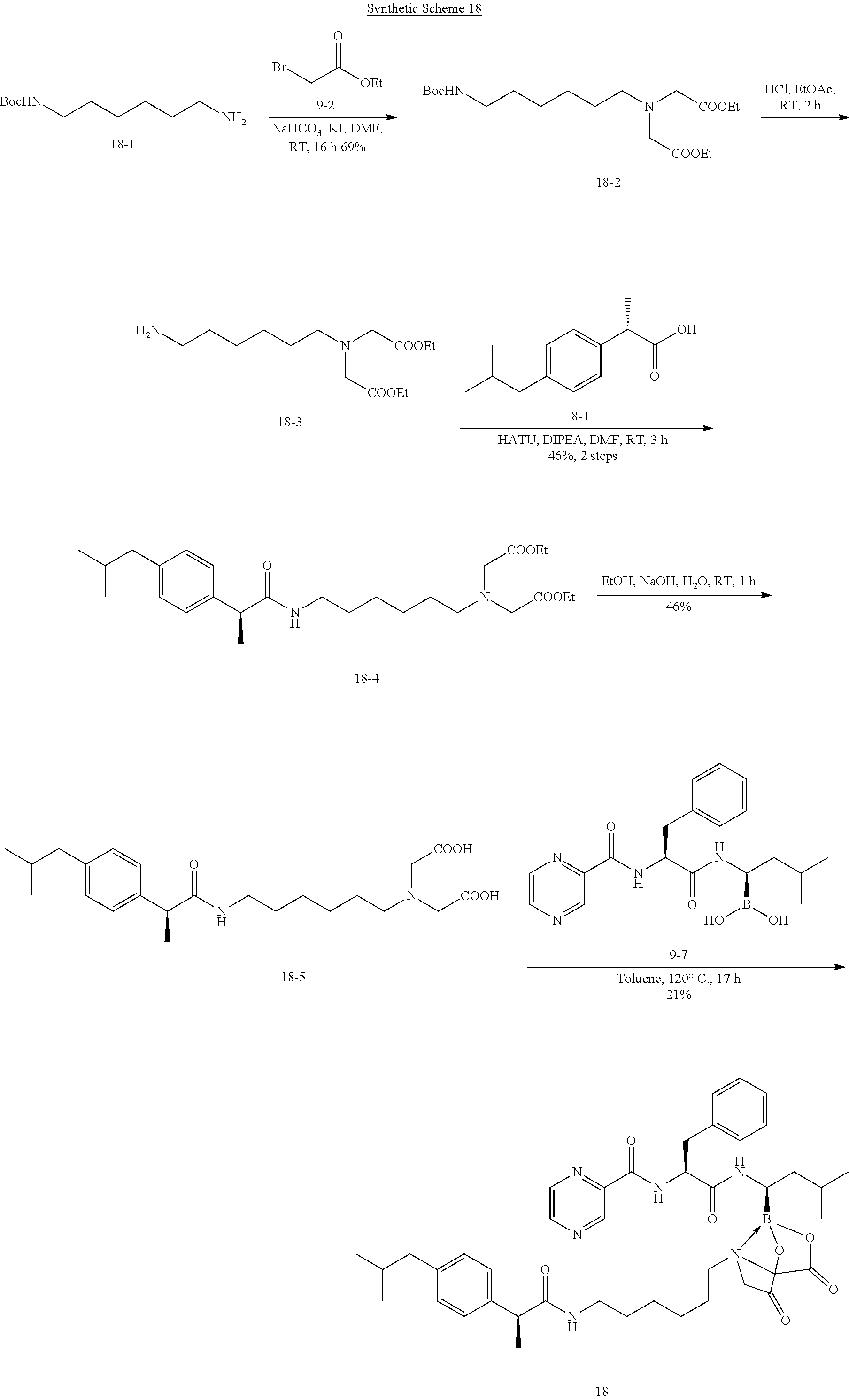

Step 1: To a solution of compound 18-1 (1.0 g, 4.62 mmol) in DMF (10 mL) was added NaHCO₃ (3.88 g, 46.18 mmol), KI (767 mg, 4.59 mmol) and compound 9-2 (3.86 g, 23.11 mmol). The mixture was stirred at room temperature for 16 h. After completion of the reaction, the mixture was partitioned between EtOAc (100 mL) and water (100 mL). The organic layer was washed with brine, dried over anhydrous Na₂SO₄ and concentrated. The residue was purified by silica gel column (eluted with 30% EtOAc in petroleum ether) to afford the title compound 18-2 (1.24 g, yield 69%).

Step 2: A mixture of compound 18-2 (700 mg, 1.80 mmol) in 4 N HCl (EtOAc solution, 7 mL) and MeOH (0.5 mL) was stirred at room temperature for 2 h. After completion of the reaction, the mixture was concentrated to afford the title compound 18-3 (622 mg, crude), which was used at the next step without further purification.

Step 3: A solution of compound 18-3 (622 mg, crude), compound 8-1 (371 mg, 1.79 mmol), DIPEA (581 mg, 4.50 mmol) and HATU (1.03 g, 2.70 mmol) in DMF (5 mL) was stirred at room temperature for 3 h. After completion of the reaction, the mixture was diluted with EtOAc (50 mL) and washed with water (50 mL×2) and brine (50 mL). The organic layer was dried by anhydrous Na₂SO₄, filtered and concentrated. The crude product was purified by silica gel column (eluted with 30% EtOAc in petroleum ether) to afford the title compound 18-4 (403 mg, yield 46% over 2 steps). LCMS: m/z calculated for C₂₇H₄₄N₂O₅: 476.66; found: 478.01 [M+H]⁺.

Step 4: To a solution of compound 18-4 (403 mg, 0.845 mmol) in EtOH (4 mL) and H₂O (2 mL) was added NaOH (135 mg, 3.37 mmol) at 0° C. and the reaction was stirred at room temperature for 1 h. The mixture was concentrated and adjusted to pH=2 with 1 N HCl at 0° C. The aqueous mixture was purified by reverse phase flash chromatography (C18 column, eluted with 50% acetonitrile in water, HCl condition). The desired components were lyophilized to afford the title compound 18-5 (181 mg, yield 46%) as a white solid. LCMS: m/z calculated for C₂₃H₃₆N₂O₅: 420.55; found: 421.76 [M+H]⁺.

Step 5: A mixture of compound 18-5 (178 mg, 0.389 mmol) and compound 9-7 (149 mg, 0.388 mmol) in toluene (3 mL) was stirred at 120° C. for 16 h. After completion of the reaction, the mixture was concentrated. The crude product was purified by pre-TLC (acetonitrile:dichloromethane=1:3) to afford 18 (65 mg, yield 21%) as a white solid. ¹H NMR (400 MHz, DMSO-d₆) δ 9.07 (d, J=1.4 Hz, 1H), 8.90 (d, J=8.4 Hz, 1H), 8.86 (d, J=2.5 Hz, 1H), 8.72 (dd, J=2.5, 1.5 Hz, 1H), 7.87 (t, J=5.5 Hz, 1H), 7.32 (d, J=9.8 Hz, 1H), 7.29-7.22 (m, 4H), 7.21-7.13 (m, 3H), 7.05 (d, J=8.0 Hz, 2H), 4.67 (td, J=8.4, 6.4 Hz, 1H), 4.16 (d, J=17.4 Hz, 1H), 4.04 (d, J=17.4 Hz, 1H), 3.94 (d, J=16.7 Hz, 1H), 3.60-3.50 (m, 2H), 3.46 (d, J=16.7 Hz, 1H), 3.09-2.95 (m, 6H), 2.38 (d, J=7.1 Hz, 2H), 1.77 (dt, J=13.5, 6.7 Hz, 1H), 1.61-1.51 (m, 2H), 1.42-1.33 (m, 3H), 1.29 (d, J=7.1 Hz, 4H), 1.26-1.15 (m, 5H), 0.83 (td, J=8.3, 6.4 Hz, 12H). LCMS: m/z calculated for C₄₂H₅₇BN₆O₇: 768.76; found: 769.93 [M+H]⁺.

### Example 19

### N-((S)-1-(((R)-1-(6-(7-((S)-2-(4-isobutylphenyl)propanamido)heptyl)-4,8-dioxo-1,3,6,2-dioxa zaborocan-2-yl)-3-methylbutyl)amino)-1-oxo-3-phenylpropan-2-yl)pyrazine-2-carboxamide (19)

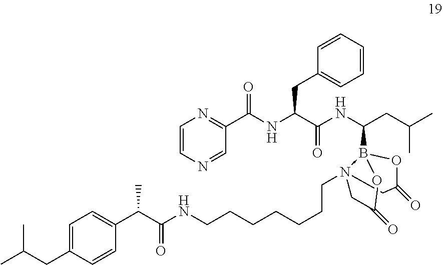 [SMILES: CC(C)Cc1ccc([C@H](C)C(=O)NCCCCCCC[N+]23CC(=O)O[B-]2([C@H](CC(C)C)NC(=O)[C@H](Cc2ccccc2)NC(=O)c2cnccn2)OC(=O)C3)cc1]

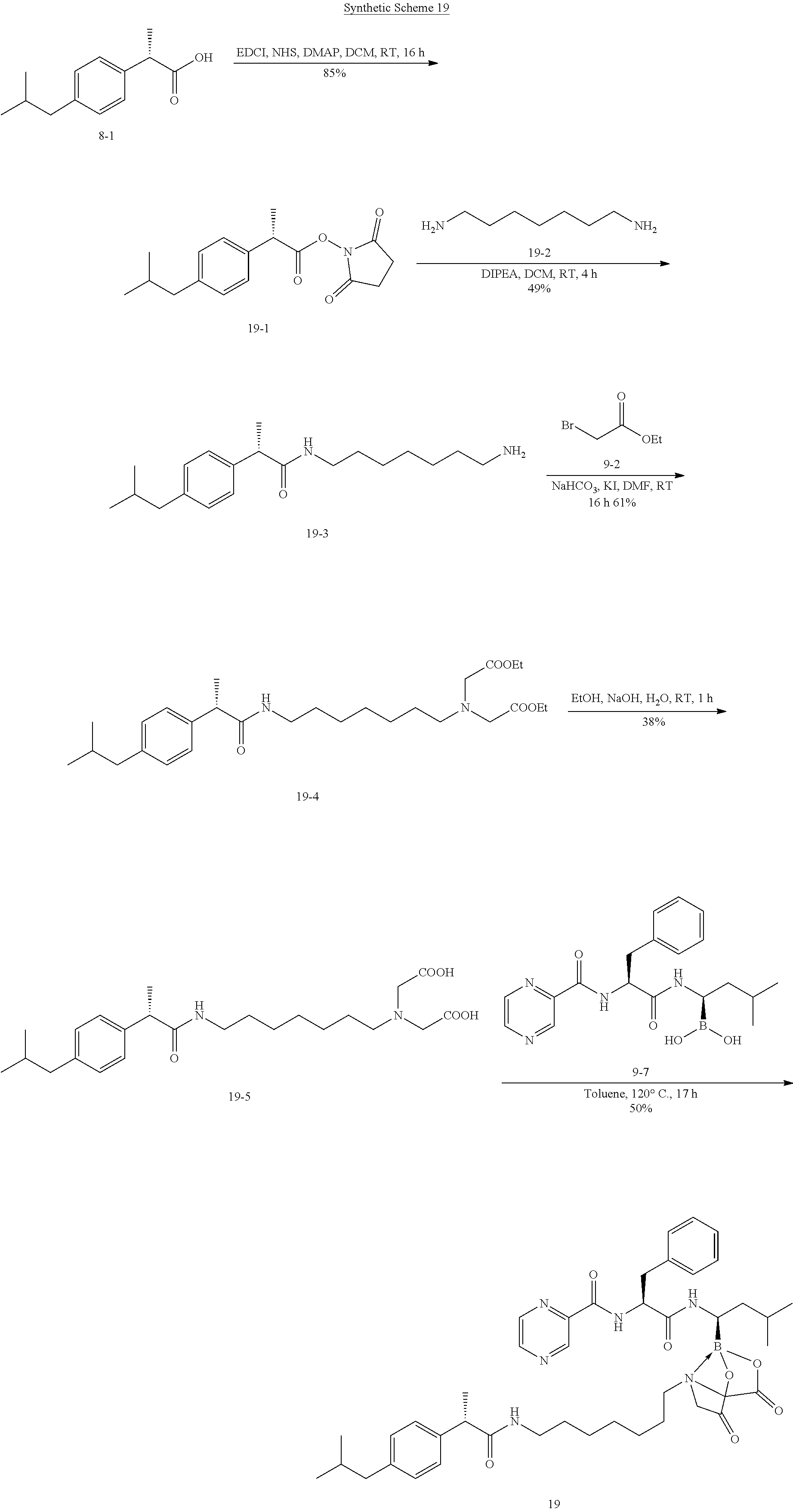

Step 1: A mixture of compound 8-1 (1 g, 4.84 mmol), NHS (7.27 mmol), DMAP (60 mg, 0.491 mmol) and EDCI (1.86 g, 9.73 mmol) in DCM (10 mL) was stirred at room temperature for 16 h. After completion of the reaction, the mixture was diluted with DCM (50 mL) and washed with 1 N HCl (50 mL) and water (50 mL). The organic layer was dried over anhydrous Na₂SO₄, filtered and concentrated to afford the title compound 19-1 (1.26 g, yield 85%) as a white solid.

Step 2: To a solution of compound 19-2 (996 mg, 7.66 mmol) and DIPEA (987 mg, 7.65 mmol) in DCM (5 mL) was added a solution of compound 19-1 (1.16 g, 3.82 mmol) in DCM (6 mL) over 1 h. After the reaction was stirred at room temperature for 4 h, the mixture was concentrated. The crude product was purified by reverse phase flash chromatography (C18 column, eluted with 80% MeOH in water, HCl condition). The desired components were concentrated to afford the title compound 19-3 (HCl salt, 667 mg, yield 49%) as a colorless oil. LCMS: m/z calculated for C₂₀H₃₄N₂O: 318.51; found: 319.88 [M+H]⁺.

Step 3: To a solution of compound 19-3 (665 mg, 2.08 mmol) in DMF (8 mL) was added NaHCO₃ (1.75 g, 20.83 mmol), KI (346 mg, 2.08 mmol) and compound 9-2 (1.74 g, 10.41 mmol). The mixture was stirred at room temperature for 16 h. After completion of the reaction, the mixture was partitioned between EtOAc (50 mL) and water (50 mL). The organic layer was washed with brine, dried over anhydrous Na₂SO₄ and concentrated. The residue was purified by silica gel column (eluted with 50% EtOAc in petroleum ether) to afford the title compound 19-4 (629 mg, yield 61%). LCMS: m/z calculated for C₂₈H₄₆N₂O₅: 490.69; found: 491.94 [M+H]⁺.

Step 4: To a solution of compound 19-4 (627 mg, 1.27 mmol) in EtOH (6 mL) and H₂O (3 mL) was added NaOH (204 mg, 5.1 mmol) at 0° C. and the reaction was stirred at room temperature for 1 h. The mixture was concentrated and adjusted to pH=2 with 1 N HCl at 0° C. The aqueous mixture was purified by reverse phase flash chromatography (C18 column, eluted with 55% acetonitrile in water, HCl condition). The desired components were lyophilized to afford the title compound 19-5 (229 mg, yield 38%) as a white solid. LCMS: m/z calculated for C₂₄H₃₈N₂O₅: 434.58; found: 435.75 [M+H]⁺.

Step 5: A mixture of compound 19-5 (195 mg, 0.414 mmol) and compound 9-7 (159 mg, 0.414 mmol) in toluene (5 mL) was stirred at 120° C. for 17 h. After completion of the reaction, the mixture was concentrated. The crude product was purified by pre-TLC (acetonitrile:dichloromethane=1:3) to afford 19 (170 mg, yield 52%) as a white solid. ¹H NMR (400 MHz, DMSO-d₆) δ 9.07 (d, J=1.5 Hz, 1H), 8.90 (d, J=8.4 Hz, 1H), 8.86 (d, J=2.5 Hz, 1H), 8.72 (q, J=1.8 Hz, 1H), 7.86 (t, J=5.6 Hz, 1H), 7.35-7.25 (m, 4H), 7.24-7.20 (m, 2H), 7.17 (d, J=10.7 Hz, 2H), 7.05 (dd, J=8.2, 2.0 Hz, 2H), 4.68 (td, J=8.3, 5.6 Hz, 1H), 4.17 (d, J=17.4 Hz, 1H), 4.04 (d, J=17.4 Hz, 1H), 3.95 (d, J=16.6 Hz, 1H), 3.60-3.50 (m, 2H), 3.47 (d, J=17.0 Hz, 1H), 3.10-2.97 (m, 6H), 2.37 (dd, J=7.2, 2.5 Hz, 2H), 1.78 (ddd, J=13.2, 6.6, 2.2 Hz, 1H), 1.56 (dp, J=10.3, 3.4 Hz, 2H), 1.42-1.33 (m, 3H), 1.31-1.11 (m, 11H), 0.86-0.80 (m, 12H). LCMS: m/z calculated for C₄₃H₅₉BN₆O₇: 782.79; found: 783.86 [M+H]⁺.

### Example 20

### N-((S)-1-(((R)-1-(6-(8-((S)-2-(4-isobutylphenyl)propanamido)octyl)-4,8-dioxo-1,3,6,2-dioxaz aborocan-2-yl)-3-methylbutyl)amino)-1-oxo-3-phenylpropan-2-yl)pyrazine-2-carboxamide (20)

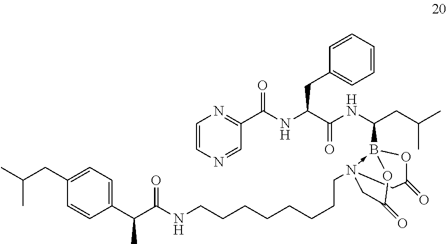 [SMILES: CC(C)Cc1ccc([C@H](C)C(=O)NCCCCCCCC[N+]23CC(=O)O[B-]2([C@H](CC(C)C)NC(=O)[C@H](Cc2ccccc2)NC(=O)c2cnccn2)OC(=O)C3)cc1]

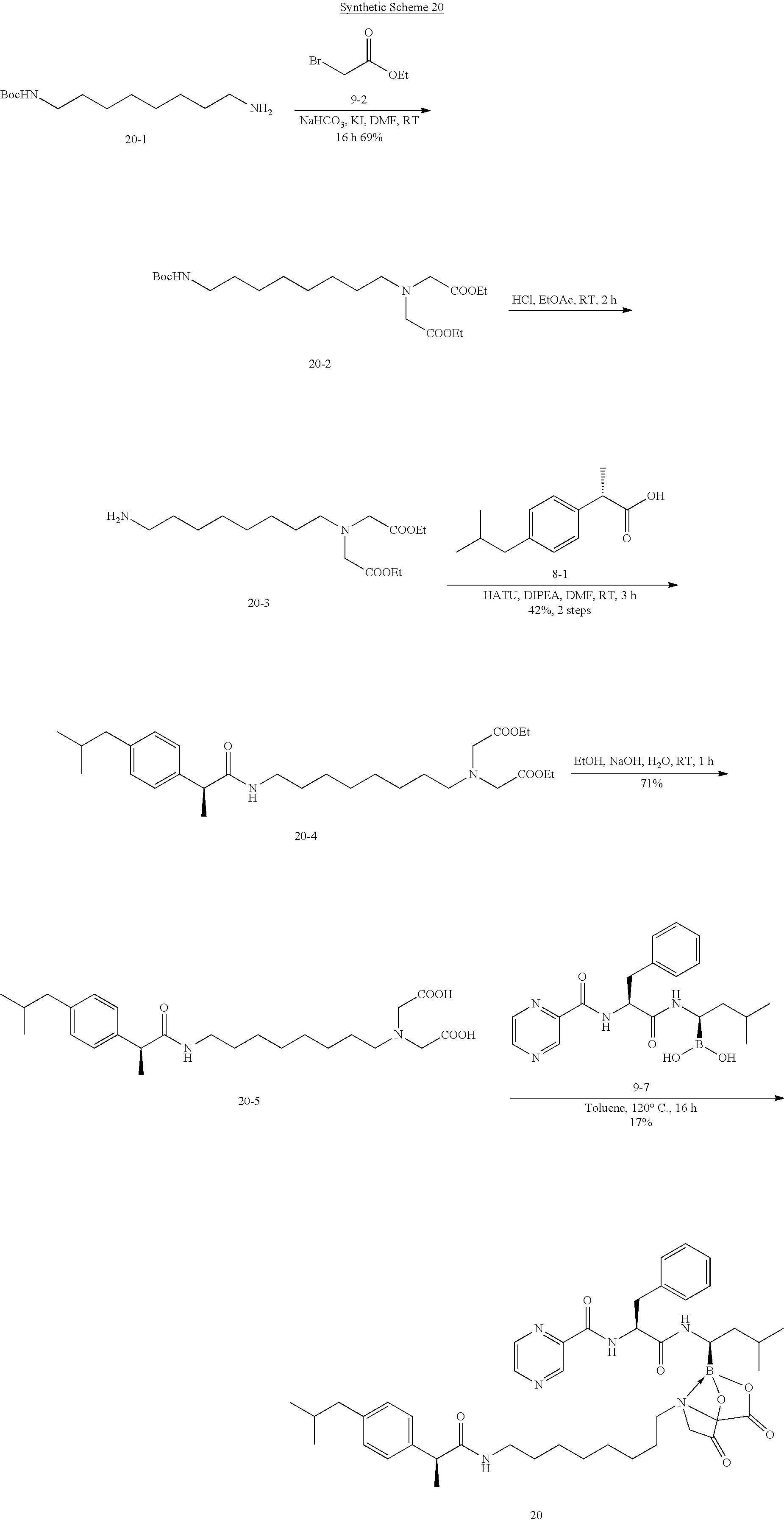

Step 1: To a solution of compound 20-1 (1.0 g, 4.09 mmol) in DMF (10 mL) was added NaHCO₃ (3.44 g, 40.94 mmol), KI (679 mg, 4.09 mmol) and compound 9-2 (3.42 g, 20.47 mmol). The mixture was stirred at room temperature for 16 h. After completion of the reaction, the mixture was partitioned between EtOAc (100 mL) and water (100 mL). The organic layer was washed with brine, dried over anhydrous Na₂SO₄ and concentrated. The residue was purified by silica gel column (eluted with 30% EtOAc in petroleum ether) to afford the title compound 20-2 (1.25 g, yield 69%).

Step 2: A mixture of compound 20-2 (700 mg, 1.68 mmol) in 4 N HCl (EtOAc solution, 7 mL) and MeOH (0.5 mL) was stirred at room temperature for 2 h. After completion of the reaction, the mixture was concentrated to afford the title compound 20-3 (637 mg, crude), which was used at next step without further purification.

Step 3: A solution of compound 20-3 (637 mg, crude from Step 2), compound 8-1 (346 mg, 1.67 mmol), DIPEA (542 mg, 4.20 mmol) and HATU (958 mg, 2.52 mmol) in DMF (5 mL) was stirred at room temperature for 16 h. After completion of the reaction, the mixture was diluted with EtOAc (50 mL) and washed with water (50 mL×2) and brine (50 mL). The organic layer was dried by anhydrous Na₂SO₄, filtered and concentrated. The crude product was purified by silica gel column (eluted with 30% EtOAc in petroleum ether) to afford the title compound 20-4 (362 mg, yield 42% over 2 steps). LCMS: m/z calculated for C₂₉H₄₈N₂O₅: 504.71; found: 506.07 [M+H]⁺.

Step 4: To a solution of compound 20-4 (360 mg, 0.713 mmol) in EtOH (4 mL) and H₂O (2 mL) was added NaOH (114 mg, 2.85 mmol) at 0° C. and the reaction was stirred at room temperature for 1 h. The mixture was concentrated and adjusted to pH=2 with 1 N HCl at 0° C. The aqueous mixture was purified by reverse phase flash chromatography (C18 column, eluted with 50% acetonitrile in water, HCl condition). The desired components were lyophilized to afford the title compound 20-5 (247 mg, yield 71%) as a white solid. LCMS: m/z calculated for C₂₅H₄₀N₂O₅: 448.60; found: 449.95 [M+H]⁺.

Step 5: A mixture of compound 20-5 (178 mg, 0.367 mmol) and compound 9-7 (141 mg, 0.366 mmol) in toluene (3 mL) was stirred at 120° C. for 16 h. After completion of the reaction, the mixture was concentrated. The crude product was purified by pre-TLC (acetonitrile:dichloromethane=1:3) to afford 20 (50 mg, yield 17%) as a white solid. ¹H NMR (400 MHz, DMSO-d₆) δ 9.07 (d, J=1.5 Hz, 1H), 8.90 (d, J=8.5 Hz, 1H), 8.86 (d, J=2.5 Hz, 1H), 8.73 (t, J=1.9 Hz, 1H), 7.85 (t, J=5.6 Hz, 1H), 7.32 (d, J=9.7 Hz, 1H), 7.29-7.23 (m, 4H), 7.23-7.17 (m, 3H), 7.05 (d, J=7.8 Hz, 2H), 4.68 (td, J=8.7, 5.8 Hz, 1H), 4.17 (d, J=17.4 Hz, 1H), 4.04 (d, J=17.4 Hz, 1H), 3.95 (d, J=16.6 Hz, 1H), 3.60-3.51 (m, 2H), 3.48 (d, J=16.6 Hz, 1H), 3.08-2.97 (m, 6H), 2.38 (d, J=7.1 Hz, 2H), 1.78 (dt, J=13.5, 6.7 Hz, 1H), 1.61-1.52 (m, 2H), 1.37 (dd, J=21.4, 4.9 Hz, 3H), 1.28 (d, J=7.1 Hz, 4H), 1.26-1.17 (m, 9H), 0.83 (q, J=8.5, 7.5 Hz, 12H). LCMS: m/z calculated for C₄₄H₆₁BN₆O₇: 796.82; found: 797.99[M+H]⁺.

### Example 21

### N-((S)-1-(((R)-1-(6-(4-(((S)-2-(4-isobutylphenyl)propanamido)methyl)cyclohexyl)-4,8-dioxo-1,3,6,2-dioxazaborocan-2-yl)-3-methylbutyl)amino)-1-oxo-3-phenylpropan-2-yl)pyrazine-2-carboxamide (21)

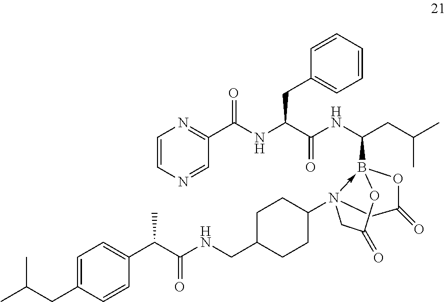 [SMILES: CC(C)Cc1ccc([C@H](C)C(=O)NCC2CCC([N+]34CC(=O)O[B-]3([C@H](CC(C)C)NC(=O)[C@H](Cc3ccccc3)NC(=O)c3cnccn3)OC(=O)C4)CC2)cc1]

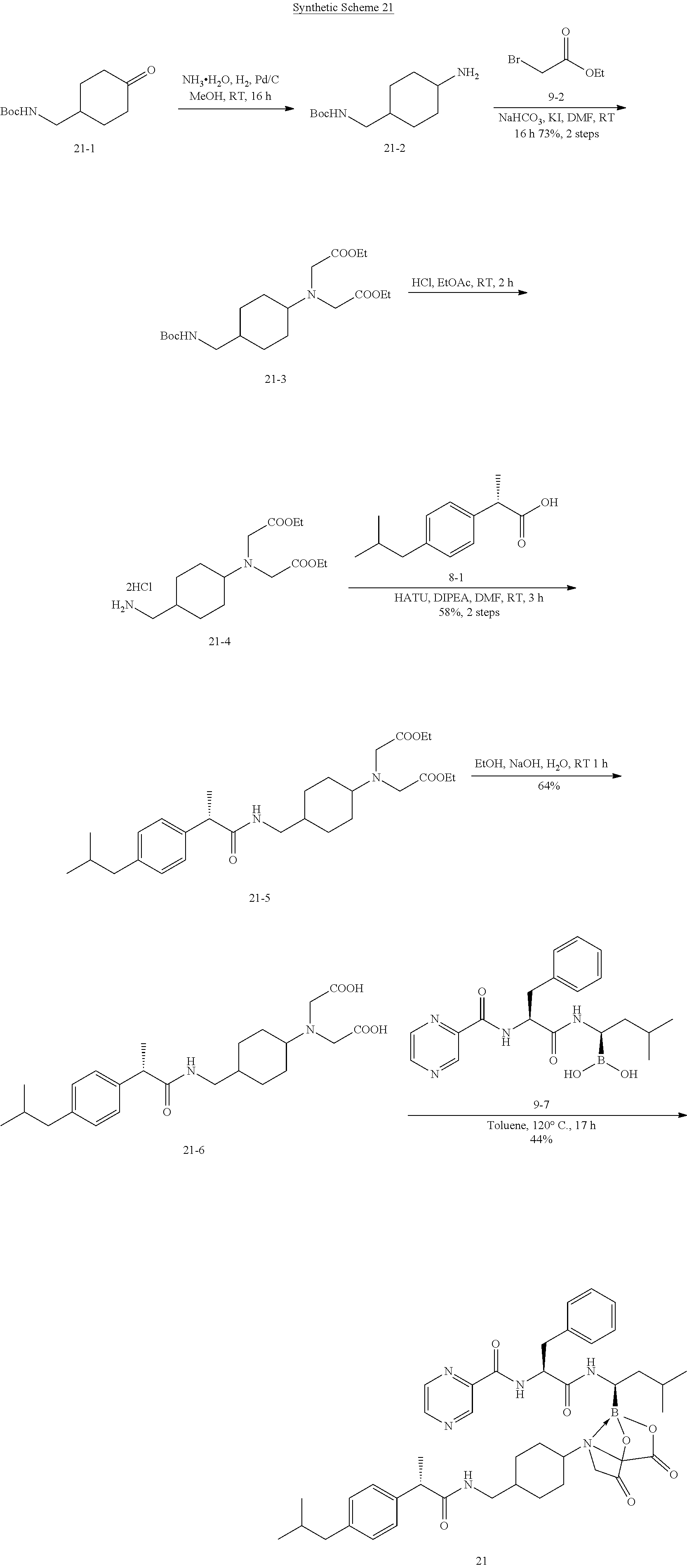

Step 1: A mixture of compound 21-1 (700 mg, 3.07 mmol), 10 w % Pd/C (350 mg) and ammonium hydroxide (28%, 9.5 mL) in MeOH (10 mL) was purged with hydrogen 3 times and stirred under hydrogen atmosphere at room temperature for 16 h. The mixture was concentrated to afford the title compound 21-2 (746 mg, crude) as a colorless oil, which was used at the next step without further purification.

Step 2: To a solution of compound 21-2 (380 mg, 1.66 mmol) in DMF (5 mL) was added NaHCO₃ (1.39 g, 16.54 mmol), KI (275 mg, 1.65 mmol) and compound 9-2 (1.38 g, 8.26 mmol). The mixture was stirred at room temperature for 16 h. After completion of the reaction, the mixture was partitioned between EtOAc (50 mL) and water (50 mL). The organic layer was washed with brine, dried over anhydrous Na₂SO₄ and concentrated. The residue was purified by silica gel column (eluted with 20% EtOAc in petroleum ether) to afford the title compound 21-3 (490 mg, yield 73% over 2 steps).

Step 3: A mixture of compound 21-3 (480 mg, 1.19 mmol) in 4 N HCl (EtOAc solution, 5 mL) was stirred at room temperature for 2 h. After completion of the reaction, the mixture was concentrated to afford the title compound 21-4 (450 mg, crude) which was used at the next step without further purification.

Step 4: A solution of compound 21-4 (450 mg), compound 8-1 (297 mg, 1.43 mmol), DIPEA (390 mg, 3.02 mmol) and HATU (684 mg, 1.79 mmol) in DMF (5 mL) was stirred at room temperature for 16 h. After completion of the reaction, the mixture was diluted with EtOAc (50 mL) and washed with water (50 mL×2) and brine (50 mL). The organic layer was dried by anhydrous Na₂SO₄, filtered and concentrated. The crude product was purified by silica gel column (eluted with 30% EtOAc in petroleum ether) to afford the title compound 21-5 (339 mg, yield 58%). ¹H NMR (400 MHz, Chloroform-d) δ 7.17 (d, J=7.1 Hz, 2H), 7.11 (d, J=7.6 Hz, 2H), 5.30 (s, 1H), 4.14 (q, J=7.1 Hz, 4H), 3.52 (s, 4H), 3.49 (d, J=7.9 Hz, 1H), 2.99 (q, J=6.0 Hz, 2H), 2.57 (t, J=11.6 Hz, 1H), 2.45 (d, J=7.0 Hz, 2H), 1.83 (d, J=8.2 Hz, 3H), 1.76 (s, 1H), 1.60 (d, J=12.7 Hz, 2H), 1.50 (d, J=7.1 Hz, 3H), 1.25 (t, J=7.1 Hz, 8H), 1.11 (q, J=12.0 Hz, 2H), 0.91-0.87 (m, 6H). LCMS: m/z calculated for C₂₈H₄₄N₂O₅: 488.67; found: 489.75 [M+H]⁺.

Step 5: To a solution of compound 21-5 (337 mg, 0.689 mmol) in EtOH (3 mL) and H₂O (1.5 mL) was added NaOH (110 mg, 2.75 mmol) at 0° C. The reaction was stirred at room temperature for 1 h. The mixture was concentrated and adjusted to pH=2 with 1 N HCl at 0° C. The aqueous mixture was purified by reverse phase flash chromatography (C18 column, eluted with 55% acetonitrile in water, HCl condition). The desired components were lyophilized to afford the title compound 21-6 (207 mg, yield 64%) as a white solid.

Step 6: A mixture of compound 21-6 (155 mg, 0.33 mmol) and compound 9-7 (127 mg, 0.33 mmol) in toluene (3 mL) was stirred at 120° C. for 16 h. After completion of the reaction, the mixture was concentrated. The crude product was purified by pre-TLC (acetonitrile:dichloromethane=1:3) to afford 21 (115 mg, yield 44%) as a off-white solid. ¹H NMR (400 MHz, DMSO-d₆) δ 9.08 (d, J=1.4 Hz, 1H), 8.89-8.85 (m, 2H), 8.73 (dd, J=2.5, 1.5 Hz, 1H), 7.92 (t, J=5.9 Hz, 1H), 7.46 (d, J=9.5 Hz, 1H), 7.32-7.28 (m, 2H), 7.25 (t, J=7.5 Hz, 2H), 7.21 (d, J=8.0 Hz, 2H), 7.19-7.15 (m, 1H), 7.05 (d, J=8.0 Hz, 2H), 4.72-4.64 (m, 1H), 4.31 (d, J=17.9 Hz, 1H), 3.84 (dd, J=17.0, 6.7 Hz, 2H), 3.62-3.52 (m, 2H), 3.25 (d, J=16.4 Hz, 2H), 3.07 (d, J=7.6 Hz, 2H), 2.86 (t, J=6.5 Hz, 2H), 2.39 (d, J=7.1 Hz, 2H), 1.77 (dt, J=13.7, 6.8 Hz, 1H), 1.74-1.62 (m, 3H), 1.61-1.36 (m, 6H), 1.31 (d, J=7.1 Hz, 4H), 1.16 (ddd, J=13.7, 10.4, 2.9 Hz, 1H), 1.09-0.97 (m, 1H), 0.86 (d, J=6.7 Hz, 3H), 0.83 (d, J=6.6 Hz, 6H), 0.78 (d, J=6.4 Hz, 3H). LCMS: m/z calculated for C₄₃H₅₇BN₆O₇: 780.77; found: 782.00 [M+H]⁺.

### Example 22

### N-((S)-1-(((R)-3-methyl-1-(6-(4-((S)-2-(4-(2-(methyl-d₃)propyl-3,3,3-d₃)phenyl)propanamid o)butyl)-4,8-dioxo-1,3,6,2-dioxazaborocan-2-yl)butyl)amino)-1-oxo-3-phenylpropan-2-yl)py razine-2-carboxamide (22)

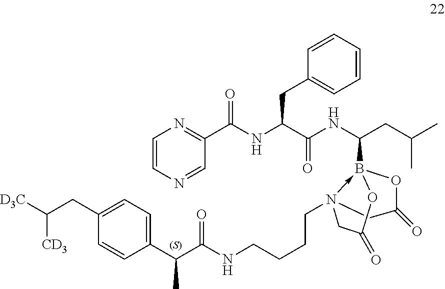

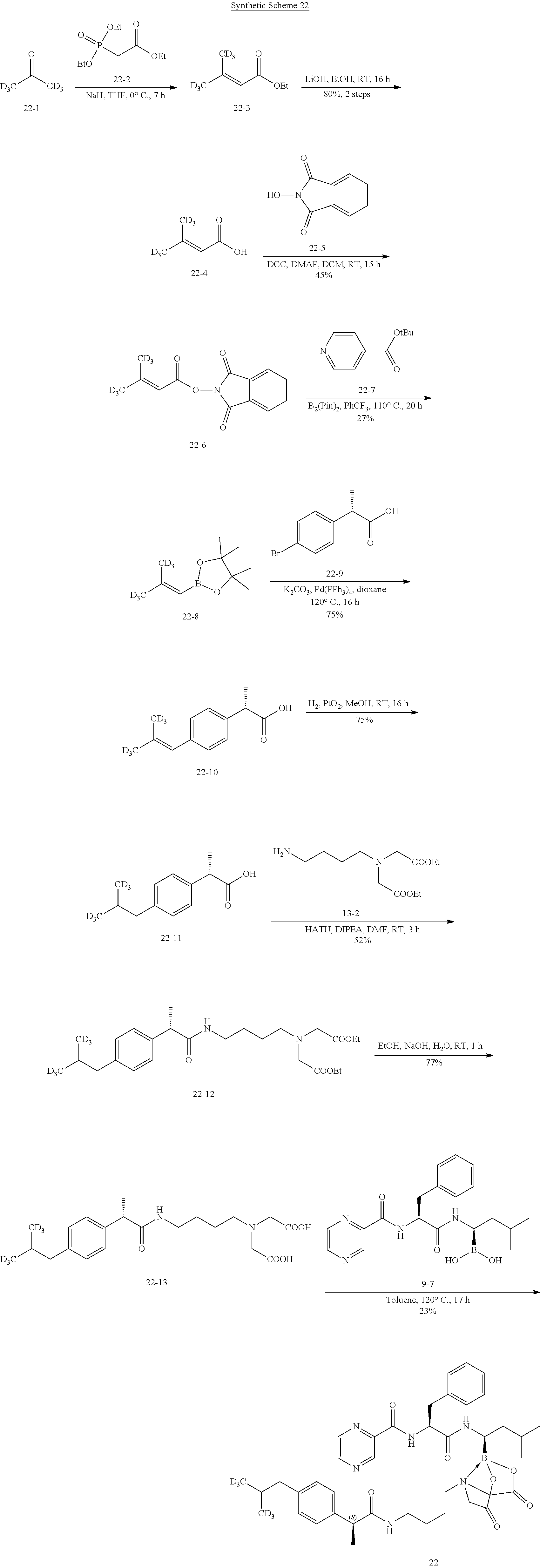

Step 1: NaH (60% in mineral oil, 9.35 g, 233 mmol) was added portion-wise over 10 min to a solution of compound 22-2 (63 g, 281 mmol) in anhydrous THF (250 mL) at 0° C. under nitrogen atmosphere. After stirring at 0° C. for 1 h, to this was added deuterated acetone 22-1 (10 g, 156 mmol) slowly over 15 min. Then the reaction was warmed to room temperature and stirred for an additional 6 h. The mixture was quenched by saturated NH₄Cl solution (300 mL) and extracted with isopropyl ether (300 mL×2). The organic layers were dried over anhydrous Na₂SO₄, filtered and concentrated to afford the title compound 22-3 (22 g, crude), which was used at the next step without further purification.

Step 2: 2 N LiOH (270 mL, 540 mmol) was added to a mixture of compound 22-3 (22 g, crude from Step 1) in EtOH (150 mL) at 0° C. The reaction was stirred at room temperature for 16 h. The resulting mixture was cooled to 0° C. and adjusted to pH=2 with 6 N aqueous HCl. The mixture was extracted with isopropyl ether (200 mL×2). The organic layers were dried over anhydrous Na₂SO₄, filtered and concentrated to afford the title compound 22-4 (13.4 g, 80% over 2 steps), which was used at the next step without further purification.

Step 3: To a solution of compound 22-4 (3 g, 28.2 mmol), compound 22-5 (6.92 g, 42.4 mmol) and DMAP (170 mg, 1.39 mmol) in DCM (50 mL) was added DCC (8.75 g, 42.4 mmol) at 0° C. The reaction was stirred at room temperature for 15 h. The mixture was concentrated and purified by silica gel column (eluted with 20% EtOAc in petroleum ether) to afford the title compound 22-6 (3.2 g, yield 45%) as a white solid.

Step 4: A mixture of compound 22-6 (2.3 g, 9.15 mmol), compound 22-7 (330 mg, 1.84 mmol) and B₂(Pin)₂ (4.65 g, 18.3 mmol) in anhydrous PhCF₃ (23 mL) was purged with nitrogen 3 times and stirred at 110° C. for 20 h. The mixture was concentrated and purified by silica gel column (eluted with 3% EtOAc in petroleum ether) to afford the title compound 22-8 (470 mg, yield 27%) as a colorless oil. ¹H NMR (400 MHz, DMSO-d₆) δ 4.98 (s, 1H), 1.19 (s, 12H).

Step 5: Under nitrogen, a mixture of compound 22-8 (550 mg, 2.92 mmol), compound 22-9 (736 mg, 3.21 mmol), Pd(PPh₃)₄ (168 mg, 0.145 mmol) and K₂CO₃ (1.2 g, 8.68 mmol) in dioxane (6 mL) was stirred at 120° C. for 16 h. After completion of the reaction, the mixture was filtered and concentrated. The crude product was purified by reverse phase flash chromatography (C18 column, eluted with 50% acetonitrile in water, TFA condition). The desired components were lyophilized to afford the title compound 22-10 (460 mg, yield 75%) as a colorless slurry.

Step 6: A mixture of compound 22-10 (460 mg, 2.18 mmol) and PtO₂ (50 mg, 0.22 mmol) in MeOH (5 mL) was stirred under hydrogen atmosphere for 16 h. After completion of the reaction, the mixture was filtered over celit and concentrated to afford the title compound 22-11 (350 mg, yield 75%) as a colorless slurry. ¹H NMR (400 MHz, DMSO-d₆) δ 7.18 (d, J=7.8 Hz, 2H), 7.08 (d, J=7.8 Hz, 2H), 3.60 (q, J=7.1 Hz, 1H), 2.40 (d, J=7.2 Hz, 2H), 1.76 (t, J=7.2 Hz, 1H), 1.32 (d, J=7.1 Hz, 3H).

Step 7: A solution of compound 13-2 (200 mg, 0.673 mmol), compound 22-11 (95 mg, 0.447 mmol), DIPEA (260 mg, 2.01 mmol) and HATU (300 mg, 0.789 mmol) in DMF (3 mL) was stirred at room temperature for 16 h. After completion of the reaction, the mixture was diluted with EtOAc (30 mL) and washed with water (30 mL×2) and brine (30 mL). The organic layer was dried by anhydrous Na₂SO₄, filtered and concentrated. The crude product was purified by silica gel column (eluted with 20% EtOAc in petroleum ether) to afford the title compound 22-12 (106 mg, yield 52%). LCMS: m/z calculated for C₂₅H₃₄D₆N₂O₅: 454.64; found: 455.99 [M+H]⁺.

Step 8: To a solution of compound 22-12 (100 mg, 0.220 mmol) in EtOH (2 mL) was added 2 N NaOH (0.44 mL, 0.88 mmol) at 0° C. and the reaction was stirred at room temperature for 1 h. The mixture was concentrated and adjusted to pH=2 with 1 N HCl at 0° C. The aqueous mixture was purified by reverse phase flash chromatography (C18 column, eluted with 35% acetonitrile in water, HCl condition). The desired components were lyophilized to afford the title compound 22-13 (68 mg, yield 75%) as a white solid.

Step 9: A mixture of compound 22-13 (60 mg, 0.151 mmol) and compound 9-7 (60 mg, 0.156 mmol) in toluene (3 mL) was stirred at 120° C. for 16 h. After completion of the reaction, the mixture was concentrated. The crude product was purified by pre-TLC (acetonitrile:dichloromethane=1:3) to afford 22 (26 mg, yield 23%) as an off-white solid. ¹H NMR (400 MHz, DMSO-d₆) δ 9.12 (s, 1H), 8.90 (s, 2H), 8.76 (s, 1H), 8.00 (s, 1H), 7.38 (d, J=9.7 Hz, 1H), 7.27 (ddt, J=24.5, 17.1, 7.2 Hz, 7H), 7.09 (d, J=7.7 Hz, 2H), 4.72 (d, J=7.9 Hz, 1H), 4.10 (q, J=17.3 Hz, 2H), 3.95 (d, J=16.6 Hz, 1H), 3.58 (d, J=8.2 Hz, 2H), 3.50 (d, J=17.1 Hz, 1H), 3.17-3.01 (m, 6H), 2.41 (d, J=7.0 Hz, 2H), 1.78 (d, J=8.4 Hz, 1H), 1.66-1.54 (m, 2H), 1.43 (d, J=12.5 Hz, 4H), 1.35 (d, J=6.8 Hz, 3H), 1.20 (s, 1H), 0.87 (dd, J=16.0, 6.7 Hz, 6H). LCMS: m/z calculated for C₄₀H₄₇D₆BN₆O₇: 746.75; found: 747.5 [M+H]⁺.

### Example 23

### N-(4-((4-amino-2-butyl-1H-imidazo[4,5-c]quinolin-1-yl)oxy)butyl)-4-(4-iodophenyl)butana mide (23)

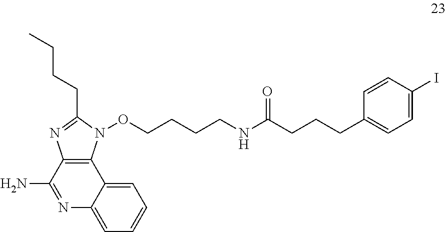 [SMILES: CCCCc1nc2c(N)nc3ccccc3c2n1OCCCCNC(=O)CCCc1ccc(I)cc1]

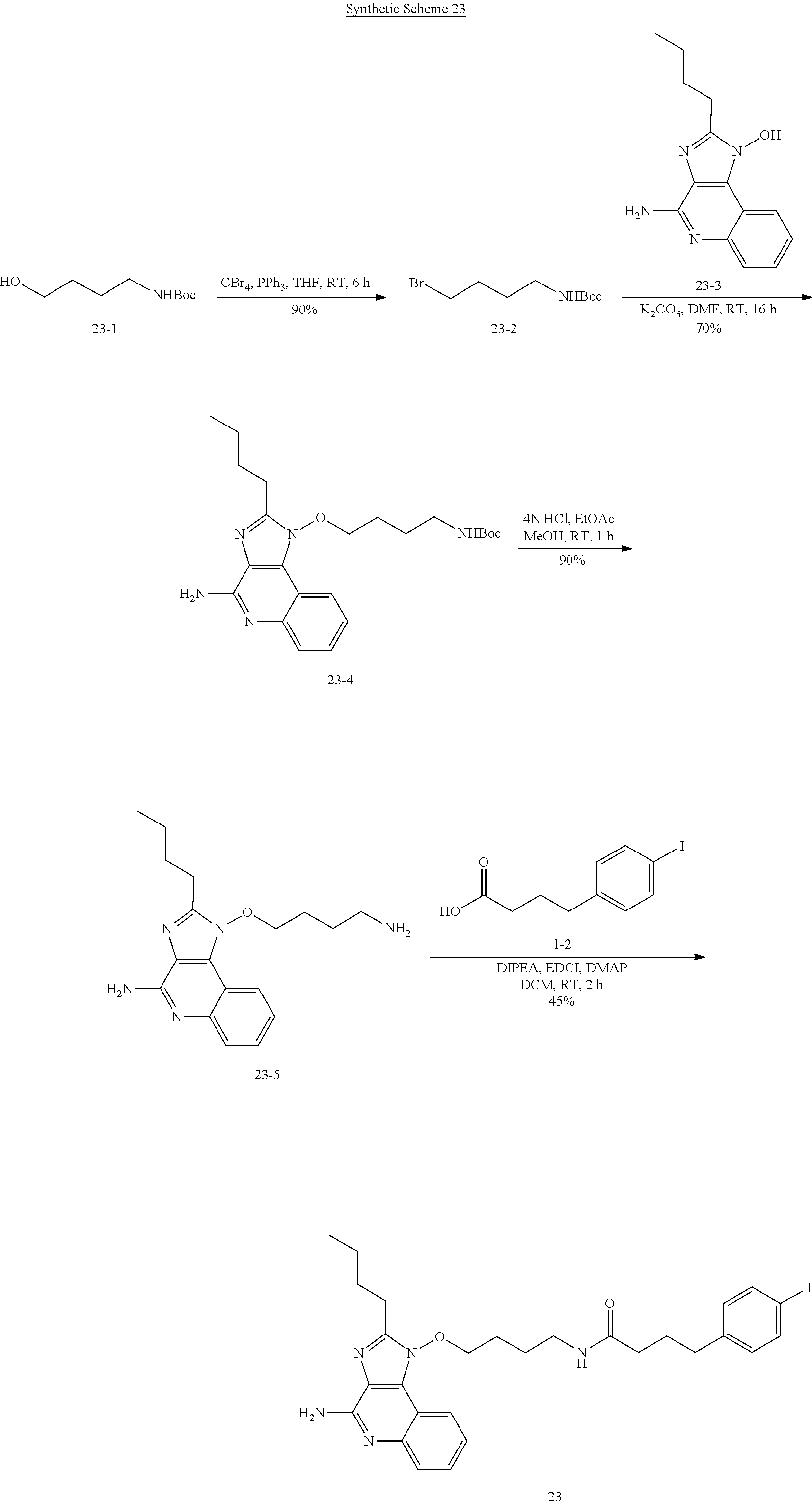

Step 1: To an ice-cold solution of compound 23-1 (10.0 g, 52.8 mmol) in anhydrous THF (200 mL) was added PPh₃ (20.8 g, 79.3 mmol), followed by a solution of CBr₄ (26.3 g, 79.3 mmol) in THF (100 mL). The reaction was allowed to warm to room temperature and stirred for 2 h. The mixture was filtered and concentrated. The residue was purified by silica gel column (eluted with 20% EtOAc in petroleum ether) to afford the title compound 23-2 (12 g, 90%) as a colorless oil.

Step 2: A mixture of compound 23-3 (500 mg, 1.95 mmol), K₂CO₃ (540 mg, 3.90 mmol) and compound 23-2 (590 mg, 2.34 mmol) in DMF (5 mL) was stirred at room temperature for 16 h. The resulting mixture was filtered and the cake washed with EtOAc (50 mL). The organic phase was washed with cold water (50 mL) and brine (50 mL). The organic layer was dried over anhydrous Na₂SO₄, filtered and concentrated. The crude product was purified by silica gel column (eluted with 5% MeOH in dichloromethane) to afford the title compound 23-4 (585 mg, yield 70%) as a pale-yellow solid.

Step 3: 4N HCl (EtOAc solution, 2.5 mL) was added to a solution of compound 23-4 (585 mg, 1.36 mmol) in EtOAc (2 mL) and MeOH (0.5 mL). After the reaction was stirred at room temperature for 1 h, the mixture was concentrated to afford the title compound 23-5 (450 mg, yield 90%) as a white solid.

Step 4: A mixture of compound 23-5 (80 mg, 0.275 mmol), DIPEA (56 mg, 0.434 mmol), DMAP (3 mg, 0.024 mmol), EDCI (65 mg, 0.340 mmol) and compound 1-2 (80 mg, 0.220 mmol) in DCM (5 mL) was stirred at room temperature for 1 h. After completion of the reaction, the mixture was concentrated. The crude product was purified by pre-HPLC (eluted with 60% acetonitrile in water, HCl condition) to afford 23 (60 mg, yield 45%) as a white solid. ¹H NMR (500 MHz, DMSO-d₆) δ 8.19 (d, J=8.1 Hz, 1H), 7.90 (d, J=5.4 Hz, 1H), 7.84 (d, J=8.4 Hz, 1H), 7.77-7.72 (m, 1H), 7.64-7.56 (m, 3H), 7.00 (d, J=8.1 Hz, 2H), 4.41 (t, J=6.6 Hz, 2H), 3.15 (q, J=6.5 Hz, 2H), 3.00 (t, J=7.6 Hz, 2H), 2.52 (d, J=7.9 Hz, 2H), 2.07 (t, J=7.4 Hz, 2H), 1.92 (t, J=7.5 Hz, 2H), 1.82 (t, J=7.6 Hz, 2H), 1.77 (t, J=7.5 Hz, 2H), 1.71-1.62 (m, 2H), 1.42 (q, J=7.4 Hz, 2H), 0.93 (t, J=7.4 Hz, 3H). LCMS: m/z calculated for C₂₈H₃₄IN₅O₂: 599.52; found: 600.82 [M+H]⁺.

### Example 24

### N-(4-((4-amino-2-butyl-7-(dimethylphosphoryl)-1H-imidazo[4,5-c]quinolin-1-yl)oxy)butyl)-4-(4-iodophenyl)butanamide (24)

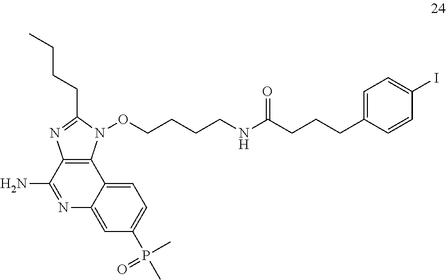 [SMILES: CCCCc1nc2c(N)nc3cc(P(C)(C)=O)ccc3c2n1OCCCCNC(=O)CCCc1ccc(I)cc1]

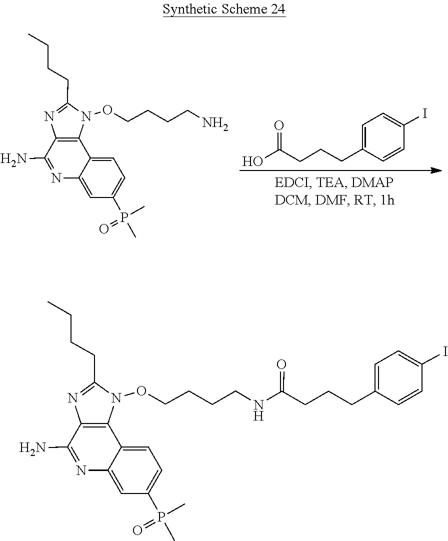 [SMILES: CCCCc1nc2c(N)nc3cc(P(C)(C)=O)ccc3c2n1OCCCCN ; O=C(O)CCCc1ccc(I)cc1 ; CCCCc1nc2c(N)nc3cc(P(C)(C)=O)ccc3c2n1OCCCCNC(=O)CCCc1ccc(I)cc1]

Step 1: A mixture of compound 24-1 (19 mg, 0.048 mmol), compound 1-2 (28 mg, 0.0965 mmol), EDCI (36 mg, 0.188 mmol), DMAP (1 mg, 0.008 mmol) and TEA (10 mg, 0.099) in DCM (0.5 ml) and DMF (0.5 ml) was stirred at room temperature for 1 h. After completion of the reaction, the resulting mixture was filtered and purified by pre-HPLC (eluted with 70% acetonitrile in water, HCl condition). The components were lyophilized to give 24 (11.2 mg, 35.8% yield) as a yellow oil. LCMS: m/z calculated for C₃₀H₃₉IN₅O₃P: 675.55; found: 676.2 [M+H]⁺.

### Example 25

### N-(2-((2-((4-((4-amino-2-butyl-1H-imidazo[4,5-c]quinolin-1-yl)oxy)butyl) (3-(dimethylamino)propyl)amino)-3,4-dioxocyclobut-1-en-1-yl)amino)ethyl)-4-(4-iodopheny l)butanamide (25)

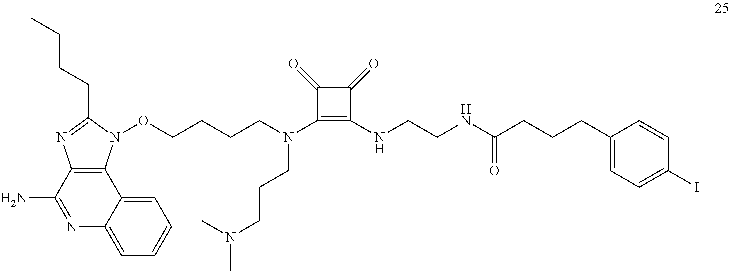 [SMILES: CCCCc1nc2c(N)nc3ccccc3c2n1OCCCCN(CCCN(C)C)c1c(NCCNC(=O)CCCc2ccc(I)cc2)c(=O)c1=O]

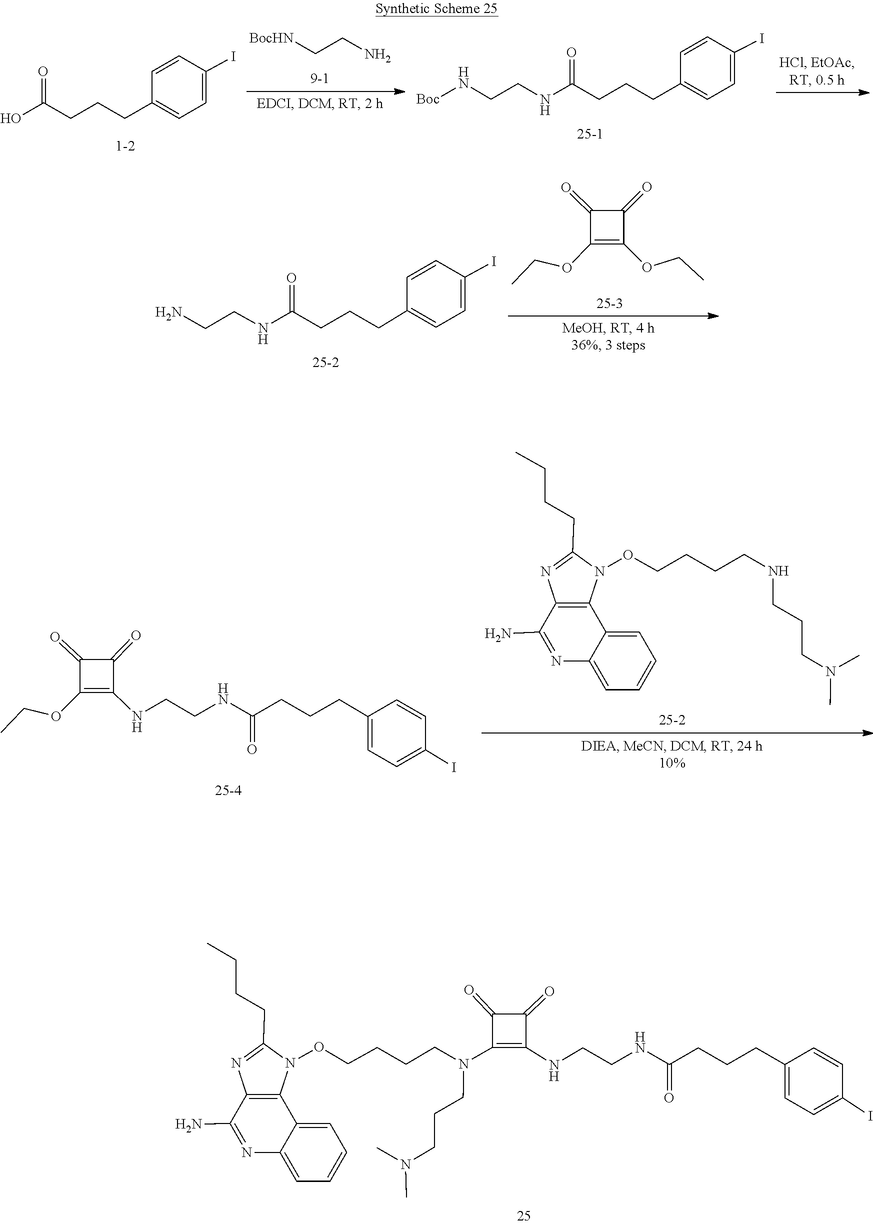

Step 1: To a solution of compound 1-2 (400 mg, 1.37 mmol) in DCM (8 mL) was added EDCI (395 mg, 2.06 mmol) and compound 9-1 (243 mg, 1.51 mmol). After stirring at RT for 2 h, TLC showed compound 1-2 was consumed. The reaction mixture was diluted with EtOAc and washed with 1 N HCl, sat. Na₂CO₃ solution and brine. The organic layer was dried over anhydrous Na₂SO₄, filtered and concentrated under reduced pressure to afford the title compound 25-1 (330 mg) as a colorless oil, which was used directly at the next step. TLC (Petroleum ether:EtOAc/1:1): R_{f} (compound 9-1)=0.55; R_{f} (compound 25-1)=0.37.

Step 2: A mixture of crude compound 25-1 (330 mg) in HCl/EtOAc (4 M, 10 mL) was stirred at RT for 30 minutes. After completion of the reaction, the mixture was concentrated. The residue was re-dissolved in EtOAc and washed with saturated Na₂CO₃ solution. The organic layer was dried over anhydrous Na₂SO₄, filtered and concentrated under reduced pressure to afford the title compound 25-2 (225 mg) as a colorless oil, which was used directly at the next step. TLC (Petroleum ether:EtOAc/1:1) R_{f} (compound 25-1)=0.37; TLC (DCM:MeOH/10:1): R_{f} (compound 25-2)=0.46.

Step 3: To a solution of compound 25-3 (154 mg, 0.905 mmol) in MeOH (5 mL) was added dropwise a solution of compound 25-2 (225 mg, crude) in MeOH (2 mL) at 0° C. After stirring at RT for 4 h, TLC showed compound 25-2 was consumed and a new spot with lower polarity was formed. The reaction mixture was evaporated in vacuum and purified by silica gel column (eluted with 5% MeOH in DCM) to afford the title compound 25-4 (230 mg, yield 36% over 3 steps) as a white solid. TLC (DCM:MeOH/10:1): R_{f} (compound 25-2)=0.46, R_{f}(compound 25-4)=0.58.

Step 4: A mixture of compound 25-4 (67 mg, 0.145 mmol), compound 25-5 (40 mg, 0.970 mmol) and DIPEA (40 mg, 0.396 mmol) in acetonitrile (2 mL) and DCM (2 mL) was stirred at RT for 24 h. LC-MS showed compound 25-5 was consumed and one new peak with desired m/z was detected by LCMS. The reaction mixture was concentrated, re-dissolved in acetonitrile and water, and purified by HPLC (C18 column, eluted with acetonitrile/H₂O, HCl condition). The desired component was lyophilized to give 25 (8 mg, yield 10%) as a white powder. ¹H NMR (400 MHz, Methanol-d₄) δ 8.12 (d, J=8.1 Hz, 1H), 7.68 (d, J=8.4 Hz, 1H), 7.53 (t, J=7.8 Hz, 1H), 7.46 (d, J=7.9 Hz, 2H), 7.36 (t, J=7.7 Hz, 1H), 6.86 (d, J=7.9 Hz, 2H), 4.33 (s, 2H), 3.75 (t, J=5.8 Hz, 2H), 3.41 (s, 2H), 3.35 (d, J=2.4 Hz, 2H), 2.99 (t, J=7.6 Hz, 2H), 2.47 (t, J=7.8 Hz, 2H), 2.39 (d, J=7.1 Hz, 2H), 2.27 (s, 6H), 2.16 (d, J=7.7 Hz, 2H), 1.98 (d, J=9.8 Hz, 2H), 1.85 (dq, J=23.9, 7.5 Hz, 8H), 1.49 (d, J=7.6 Hz, 2H), 1.30-1.26 (m, 2H), 1.01 (t, J=7.4 Hz, 3H). LCMS: m/z calculated for C₃₉H₅₁IN₈O₄: 822.79; found: 823.4 [M+H]⁺.

### Example 26

### (S)-N-(2-((2-((4-((4-amino-2-butyl-1H-imidazo[4,5-c]quinolin-1-yl)oxy)butyl)amino)-3,4-dio xocyclobut-1-en-1-yl)amino)ethyl)-2-(4-isobutylphenyl)propanamide (26)

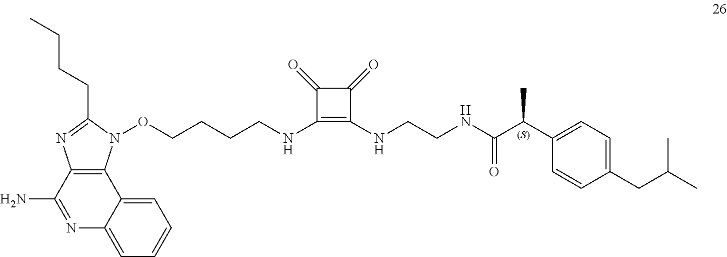

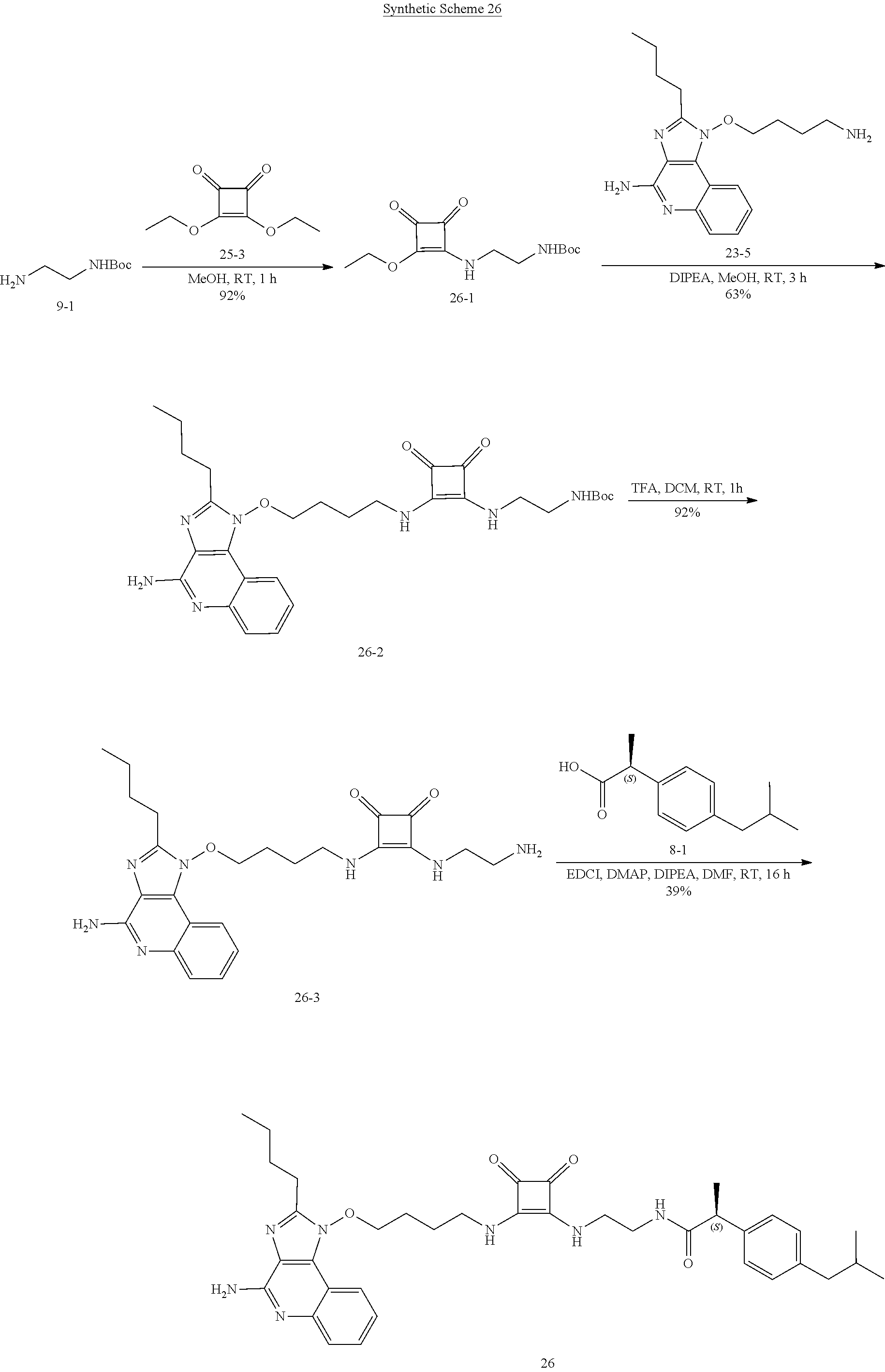

Step 1: To a solution of compound 25-3 (1.06 g, 6.23 mmol) in anhydrous MeOH (10 mL) was added dropwise a solution of compound 9-1 (1 g, 6.24 mmol) in anhydrous MeOH (10 mL). The mixture was stirred at room temperature for 1 h. After completion of the reaction, the mixture was diluted with EtOAc (50 mL), washed with water (50 mL) and brine (50 mL). The organic layer was dried over anhydrous Na₂SO₄, filtered and concentrated. The crude product was purified by column (eluted with 40% EtOAc in petroleum ether) to afford the title compound 26-1 (1.63 g, yield 97%) as a colorless oil.

Step 2: To a solution of compound 23-5 (339 mg, 1.03 mmol) and DIPEA (333 mg 2.58 mmol) in anhydrous MeOH (4 mL) was added dropwise a solution of compound 26-1 (440 mg, 1.55 mmol) in anhydrous MeOH (3 mL). The mixture was stirred at room temperature for 3 h. After completion of the reaction, the mixture was concentrated. The crude product was purified by reverse phase flash chromatography (C18 column, eluted with 60% acetonitrile in water, TFA condition). The desired components were lyophilized to give the title compound 26-2 (TFA salt, 445 mg, yield 63%) as a yellow solid.

Step 3: A mixture of compound 26-2 (445 mg, 0.65 mmol) in a solution of 20% TFA in DCM (3 mL) was stirred at room temperature for 1 h. After completion of the reaction, the mixture was concentrated to get rid of organic solvents to afford the title compound 26-3 (420 mg, yield 92%) as a pale-yellow slurry.

Step 4: A mixture of compound 26-3 (100 mg, 0.148 mmol), compound 8-1 (46 mg, 0.222 mmol), DMAP (2 mg, 0.016 mmol), DIPEA (57 mg, 0.442 mmol) and EDCI (42 mg, 0.220 mmol) in DMF (2 mL) was stirred at room temperature for 16 h. After completion of the reaction, the mixture was filtered and purified by pre-HPLC (eluted with 50% acetonitrile in water, HCl condition). The desired components were lyophilized to afford 26 (40 mg, 39% yield) as a white solid. ¹H NMR (600 MHz, DMSO-d₆) δ 8.20 (dd, J=8.1, 1.4 Hz, 1H), 7.85-7.81 (m, 1H), 7.74 (ddd, J=8.4, 7.2, 1.3 Hz, 1H), 7.62 (t, J=7.7 Hz, 1H), 7.17 (d, J=7.5 Hz, 2H), 7.01 (d, J=7.2 Hz, 2H), 4.44 (t, J=6.5 Hz, 2H), 3.57-3.43 (m, 5H), 3.28 (d, J=7.0 Hz, 1H), 3.12 (dd, J=13.3, 6.5 Hz, 1H), 3.00 (t, J=7.6 Hz, 2H), 2.39-2.31 (m, 2H), 2.01 (s, 2H), 1.82 (td, J=14.1, 13.2, 6.6 Hz, 4H), 1.76 (d, J=4.4 Hz, 1H), 1.43 (q, J=7.4 Hz, 2H), 1.29 (d, J=7.0 Hz, 3H), 0.93 (t, J=7.4 Hz, 3H), 0.81 (d, J=6.6 Hz, 6H). LCMS: m/z calculated for C₃₇H₄₇N₇O₄: 653.83; found: 654.88 [M+H]⁺.

### Example 27

### (S)-N-(4-((4-amino-2-butyl-7-(dimethylphosphoryl)-1H-imidazo[4,5-c]quinolin-1-yl)oxy)but yl)-2-(4-isobutylphenyl)propanamide (27)

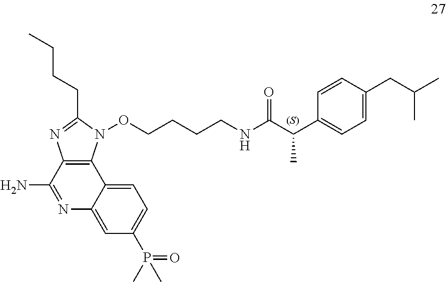

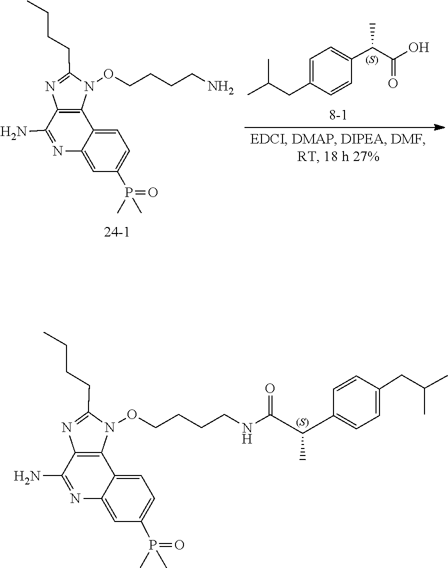

Step 1: A mixture of compound 24-1 (60 mg, 0.148 mmol), compound 8-1 (46 mg, 0.223 mmol), DMAP (2 mg, 0.016 mmol), DIPEA (57 mg, 0.442 mmol) and EDCI (45 mg, 0.235 mmol) in DMF (2 mL) was stirred at room temperature for 16 h. After completion of the reaction, the mixture was filtered and purified by pre-HPLC (eluted with 45% acetonitrile in water, HCl condition). The desired components were lyophilized to afford 27 (24 mg, 27% yield) as a white solid. ¹H NMR (600 MHz, DMSO-d₆) δ 8.29-8.24 (m, 2H), 8.09 (t, J=5.7 Hz, 1H), 7.95 (ddd, J=10.0, 8.3, 1.3 Hz, 1H), 7.23 (d, J=8.1 Hz, 2H), 7.05 (d, J=8.1 Hz, 2H), 4.39 (t, J=6.6 Hz, 2H), 3.60 (s, 1H), 3.16 (q, J=6.6 Hz, 2H), 3.04-3.00 (m, 2H), 2.37 (d, J=7.1 Hz, 2H), 1.91-1.82 (m, 4H), 1.77 (d, J=13.5 Hz, 7H), 1.68-1.63 (m, 2H), 1.49-1.41 (m, 2H), 1.32 (d, J=7.1 Hz, 3H), 0.96 (t, J=7.4 Hz, 3H), 0.82 (s, 6H). LCMS: m/z calculated for C₃₃H₄₆N₅O₃P: 591.74; found: 592.93 [M+H]⁺.

### Example 28

### (5-fluoro-2,4-dioxo-3,4-dihydropyrimidin-1(2H)-yl)methyl 4-(4-iodophenyl) butanoate (28)

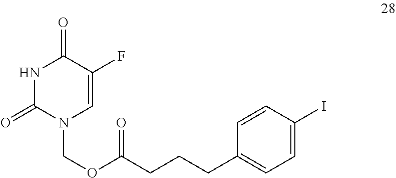 [SMILES: O=C(CCCc1ccc(I)cc1)OCn1cc(F)c(=O)[nH]c1=O]

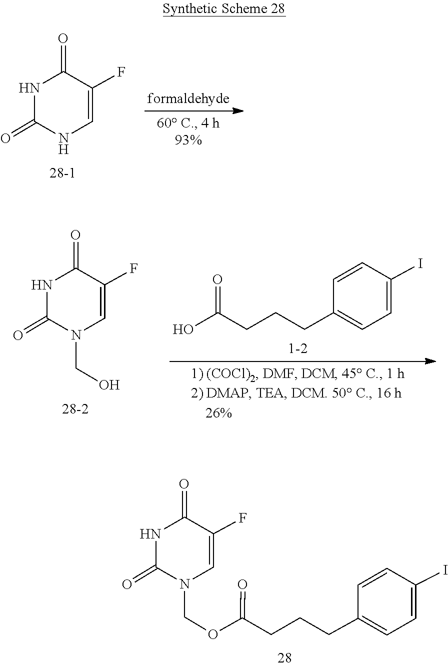 [SMILES: O=c1[nH]cc(F)c(=O)[nH]1 ; O=c1[nH]c(=O)n(CO)cc1F ; O=C(O)CCCc1ccc(I)cc1 ; O=C(CCCc1ccc(I)cc1)OCn1cc(F)c(=O)[nH]c1=O]

Step 1: Compound 28-1 (1.3 g, 10.0 mmol) was dissolved in 37% formaldehyde solution (1.8 g, 22.2 mmol) and the mixture was refluxed with stirring at 60° C. for 4 h. The mixture was concentrated to give the title compound 28-2 (1.5 g, yield 93%) as a colorless oil, which was used directly at the next step without purification.

Step 2: Oxalyl chloride (0.50 mL, 5.82 mmol) and catalytic amount DMF were added to a solution of compound 1-2 (200 mg, 0.69 mmol) in dichloromethane (20 mL). The resulting mixture was stirred at 45° C. for 1 h. The mixture was azeotroped with dichloromethane3 times under reduced pressure to give the crude chloride intermediate. The chloride was re-dissolved in anhydrous dichloromethane (5 mL) and added to a solution of compound 28-2 (220 mg, 1.38 mmol), DMAP (8 mg, 0.07 mmol) and TEA (100 uL, 0.69 mmol) in anhydrous dichloromethane (10 mL) at room temperature. The resulting mixture was refluxed at 50° C. with stirring for 10 h. After the mixture was concentrated, the residue was purified by silica gel column (eluted with 50% EtOAc in petroleum ether) to afford 28 (72 mg, 26% yield) as a white solid. ¹H NMR (400 MHz, DMSO-d6) δ 12.00 (s, 1H), 8.14 (d, J=6.4 Hz, 1H), 7.68-7.56 (m, 2H), 7.01 (d, J=7.8 Hz, 2H), 5.56 (s, 2H), 2.54 (d, J=7.6 Hz, 2H), 2.33 (t, J=7.3 Hz, 2H), 1.78 (t, J=7.5 Hz, 2H).

### Example 29

### (5-fluoro-2,4-dioxopyrimidine-1,3(2H,4H)-diyl)bis(methylene)bis(4-(4-iodo-1-phenyl)butanoate) (29)

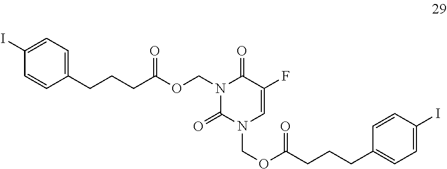 [SMILES: O=C(CCCc1ccc(I)cc1)OCn1cc(F)c(=O)n(COC(=O)CCCc2ccc(I)cc2)c1=O]

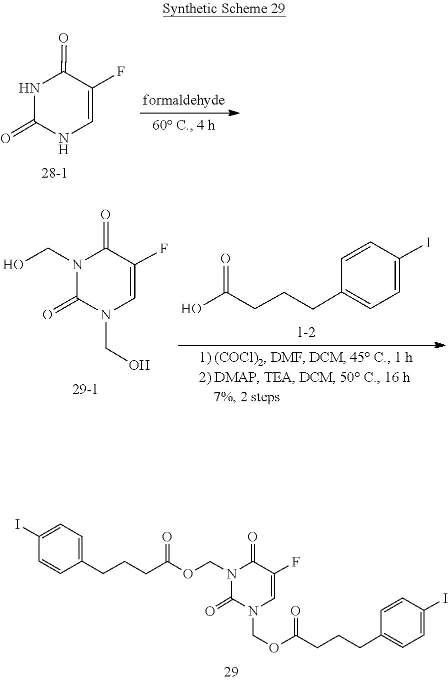 [SMILES: O=c1[nH]cc(F)c(=O)[nH]1 ; O=c1c(F)cn(CO)c(=O)n1CO ; O=C(O)CCCc1ccc(I)cc1 ; O=C(CCCc1ccc(I)cc1)OCn1cc(F)c(=O)n(COC(=O)CCCc2ccc(I)cc2)c1=O]

Step 1: A suspension of compound 28-1 (1.0 g, 7.69 mmol) in 37% formaldehyde solution (2 mL) was stirred at 60° C. for 4 h. The reaction mixture was concentrated to afford the title compound 29-1 (1.5 g) as a colorless oil, which was used directly at the next step without purification.

Step 2: To a solution of compound 1-2 (500 mg, 1.73 mmol) in DCM (5 mL) was added oxalyl chloride (2.18 g, 17.21 mmol) and catalytic amount DMF at room temperature. The resulting mixture was stirred at 45° C. for 1 h. After compound 1-2 was consumed, the mixture was concentrated. The residue was re-dissolved in DCM (10 mL), to this solution was added compound 29-1 (250 mg, 1.32 mmol), DMAP (16 mg, 0.131 mmol) and TEA (650 mg, 6.44 mmol) at room temperature. The resulting mixture was stirred at 50° C. for 16 h. After completion of the reaction, the mixture was concentrated. The residue was purified by silica gel column (eluted with 2% EtOAc in petroleum ether) and further purified by reverse phase flash chromatography (C18 column, eluted with acetonitrile and water, TFA condition) to afford 29 (70 mg, 7% yield over 2 steps) as a white solid. ¹H NMR (400 MHz, DMSO-d₆) δ 8.29 (dq, J=6.3, 3.8, 3.1 Hz, 1H), 7.61 (ddq, J=6.4, 4.1, 2.4 Hz, 4H), 6.99 (dt, J=8.1, 3.4 Hz, 4H), 5.82-5.75 (m, 2H), 5.67-5.58 (m, 2H), 2.52 (d, J=5.9 Hz, 4H), 2.35-2.23 (m, 4H), 1.76 (dq, J=12.2, 7.2 Hz, 4H). LCMS: m/z calculated for C₂₆H₂₅FI₂N₂O₆: 734.30; found: 757.33. [M+Na]⁺.

### Example 30

### (2aR,4S,4aS,6R,9S,11 S,12S,12aR,12bS)-12b-acetoxy-9-(((2R,3S)-3-((tert-butoxycarbonyl)amino)-2-((4-(4-iodophenyl)butanoyl)oxy)-3-phenylpropanoyl)oxy)-4,6,11-trihydroxy-4a,8,13,13-tetramethyl-5-oxo-2a,3,4,4a,5,6,9,10,11,12,12a,12b-dodecahydro-1H-7,11-methanocyclodeca[3,4]benzo[1,2-b]oxet-12-yl benzoate (30)

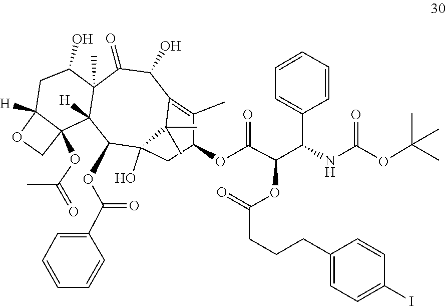

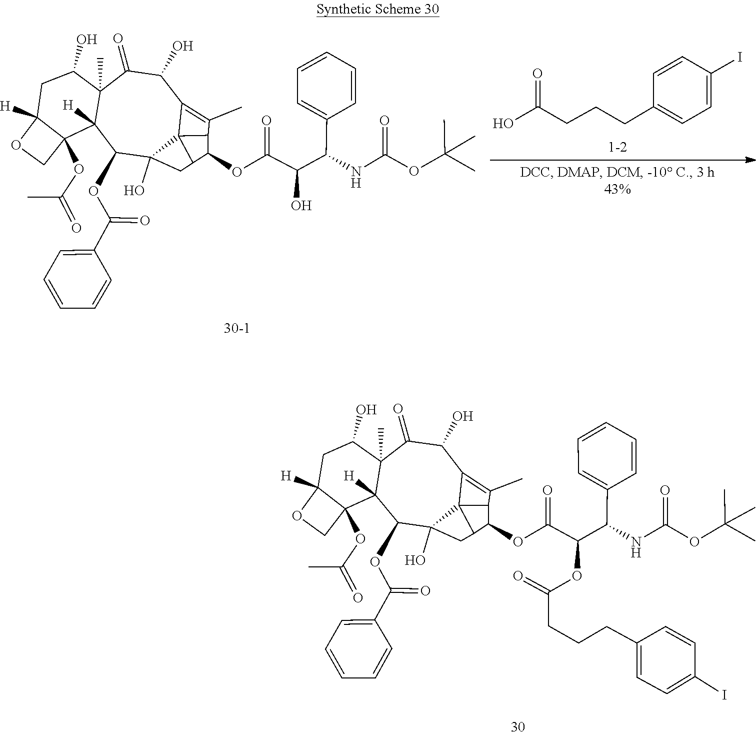

Step 1: DCC (77 mg, 0.37 mmol) and DMAP (45 mg, 0.37 mmol) were added to a solution of compound 30-1 (200 mg, 0.25 mmol) and 4-(p-iodophenyl)butyric acid 1-2 (79 mg, 0.27 mmol) in dichloromethane (16 mL) at −10° C. After stirring at −10° C. for 3 h, the mixture was filtered, diluted with EtOAc (50 mL), and washed with water (50 mL) and brine (50 mL). The organic layer was dried over anhydrous Na₂SO₄ and concentrated. The residue was purified by silica gel column (eluted with 40% EtOAc in petroleum ether) to afford 30 (115 mg, yield: 43%) as a white solid. ¹H NMR (400 MHz, Chloroform-d) δ 8.16 (d, J=7.7 Hz, 2H), 7.68-7.60 (m, 3H), 7.55 (t, J=7.5 Hz, 2H), 7.43 (t, J=7.5 Hz, 2H), 7.37-7.31 (m, 3H), 6.90 (d, J=7.8 Hz, 2H), 6.30 (s, 1H), 5.74 (d, J=7.0 Hz, 1H), 5.52 (s, 1H), 5.45-5.34 (m, 2H), 5.26 (s, 1H), 5.01 (d, J=9.4 Hz, 1H), 4.37 (d, J=8.5 Hz, 1H), 4.31 (q, J=8.3 Hz, 1H), 4.24 (d, J=6.4 Hz, 2H), 4.20-4.13 (m, 1H), 3.99 (d, J=7.0 Hz, 1H), 2.64 (dt, J=15.4, 8.1 Hz, 1H), 2.54 (t, J=7.6 Hz, 3H), 2.49 (s, 3H), 2.38 (dp, J=23.3, 8.3 Hz, 4H), 2.22 (s, 1H), 2.09 (d, J=1.7 Hz, 2H), 2.01 (s, 3H), 1.90 (q, J=8.4, 7.7 Hz, 4H), 1.80 (s, 3H), 1.69 (s, 1H), 1.63 (d, J=1.7 Hz, 3H), 1.54 (d, J=7.5 Hz, 1H), 1.38 (s, 11H), 1.32 (d, J=1.9 Hz, 1H), 1.30 (d, J=1.7 Hz, 2H), 1.28 (s, 4H), 1.17 (s, 4H). LCMS: m/z calculated for C₅₃H₆₂INO₁₅: 1079.98; found: 1102.7 [M+Na]⁺.

### Example 31

### N²-(4-(2-(2-amino-4-oxo-4,7-dihydro-31H-pyrrolo[2,3-d]pyrimidin-5-yl)ethyl) benzoyl)-N⁵-(2-(4-(4-iodophenyl)butanamido)ethyl)-L-glutamine (31)

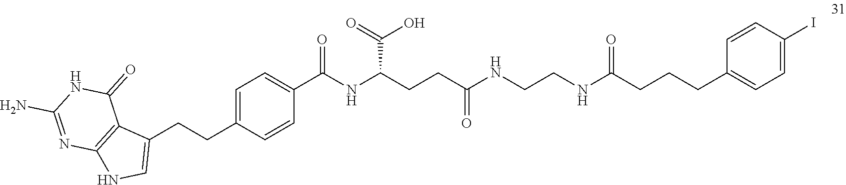 [SMILES: Nc1nc2[nH]cc(CCc3ccc(C(=O)N[C@@H](CCC(=O)NCCNC(=O)CCCc4ccc(I)cc4)C(=O)O)cc3)c2c(=O)[nH]1]

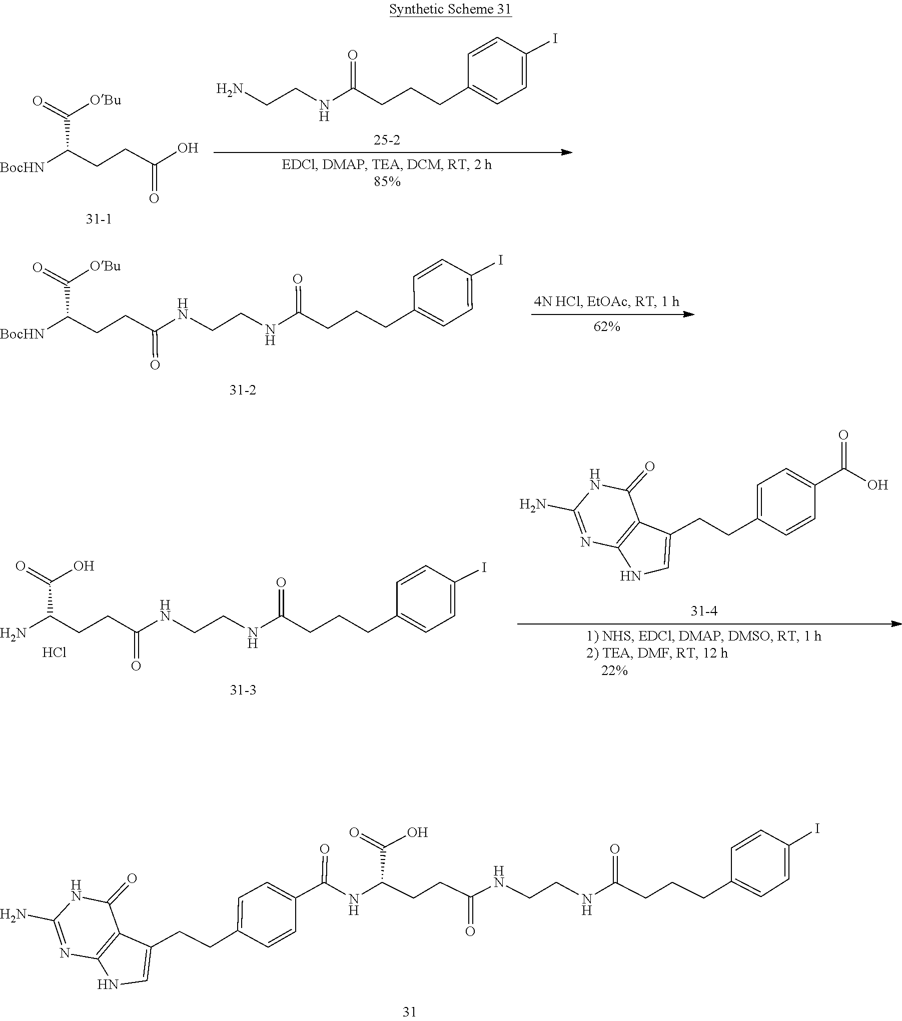

Step 1: EDCI (362 mg, 1.89 mmol), triethylamine (0.74 mL, 5.16 mmol) and DMAP (20 mg, 0.17 mmol) were added to a solution of compound 31-1 (520 mg, 1.41 mmol) and compound 25-2 (574 mg, 1.89 mmol) in dichloromethane (20 mL) at 0° C. The reaction was stirred at room temperature for 2 h, the mixture was diluted with dichloromethane (100 mL), washed with 1N HCl (100 mL) and brine (100 mL). The organic layer was dried over anhydrous Na₂SO₄, filtered and concentrated to afford the title compound 31-2 (740 mg, yield: 85%), which was used at the next step without further purification.

Step 2: A mixture of compound 31-2 (400 mg, 0.65 mmol) in 4 N HCl (EtOAc solution, 5 mL) was stirred at room temperature for 1 h. A white precipitation was formed and filtered. The solid was dried in vacuum to afford the title compound 31-3 (HCl salt, 200 mg, yield 62%).

Step 3: A solution of compound 31-4 (200 mg, 0.67 mmol), DMAP (8 mg, 0.07 mmol), NHS (115 mg, 1.00 mmol) and EDCI (128 mg, 0.67 mmol) in anhydrous DMSO (3 mL) was stirred at room temperature for 1 h. Compound 31-3 (200 mg, 0.40 mmol) and Et₃N (483 uL, 3.35 mmol) were added. After stirring at room temperature for 12 h, the mixture was purified by prep-HPLC (eluted with 60% acetonitrile in water, HCl condition) to give 31 (67 mg, yield: 22%) as a light pink solid. ¹H NMR (400 MHz, DMSO-d6) δ 10.71 (s, 1H), 8.60 (d, J=7.7 Hz, 1H), 7.89 (s, 1H), 7.79 (d, J=7.7 Hz, 3H), 7.61 (d, J=7.9 Hz, 2H), 7.29 (d, J=7.9 Hz, 2H), 7.00 (d, J=8.0 Hz, 2H), 6.34 (s, 1H), 6.27 (s, 1H), 4.32 (d, J=10.2 Hz, 1H), 3.12-2.80 (m, 12H), 2.11 (dt, J=69.0, 7.7 Hz, 7H), 1.79-1.67 (m, 2H). LCMS: m/z calculated for C₃₂H₃₆IN₇O₆: 741.59; found: 742.4. [M+H]⁺.

### Example 32

### (S)-2-(4-(2-(2-amino-4-oxo-4,7-dihydro-3H-pyrrolo[2,3-d]pyrimidin-5-yl)ethyl) benzamido)-5-(2-(4-(4-iodophenyl)butanamido)ethoxy)-5-oxopentanoic acid (32)

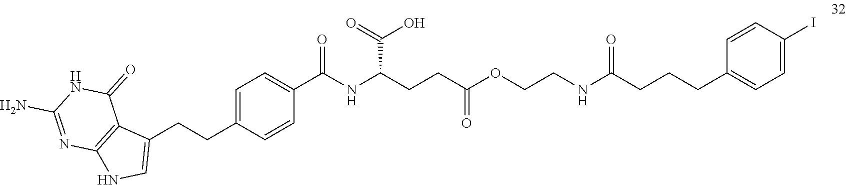 [SMILES: Nc1nc2[nH]cc(CCc3ccc(C(=O)N[C@@H](CCC(=O)OCCNC(=O)CCCc4ccc(I)cc4)C(=O)O)cc3)c2c(=O)[nH]1]

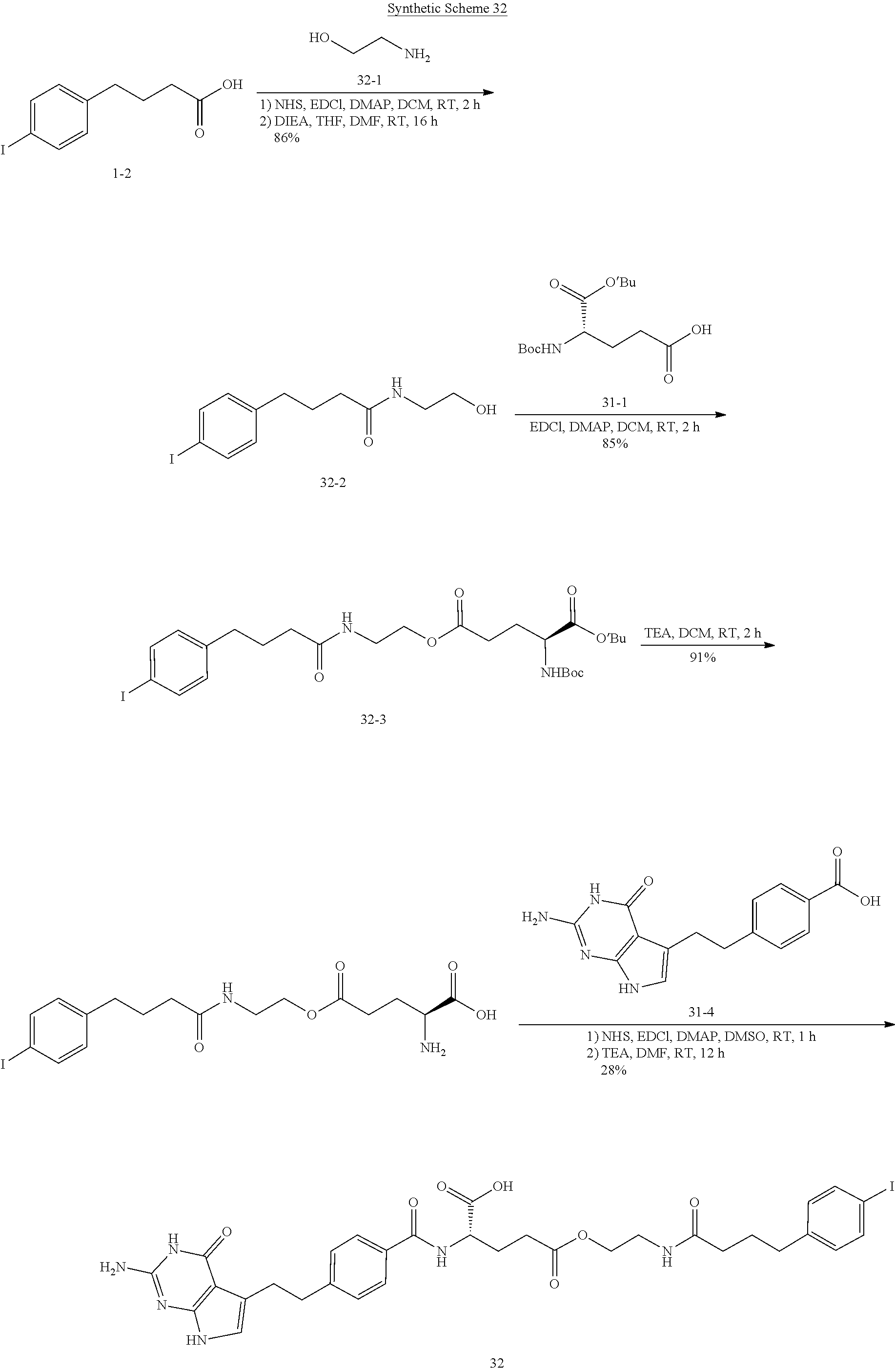

Step 1: To a mixture of acid 1-2 (500 mg, 1.72 mmol) and NHS (295 mg, 2.56 mmol) in DCM (10 mL) were added EDCI (658 mg, 3.44 mmol) and DMAP (21 mg, 0.172 mmol), the mixture was stirred at room temperature for 2 h. The reaction mixture was diluted with DCM (50 mL) and washed with 1 N HCl (50 mL). The organic layer was dried over anhydrous Na₂SO₄, filtered and concentrated to give the crude activated NHS ester as a white solid. To a solution of compound 32-1 (126 mg, 2.06 mmol) and DIEA (340 uL, 2.06 mmol) in DMF (5 mL) was added dropwise a solution of activated NHS ester in THF (5 mL). The reaction mixture was allowed to stir at room temperature for 16 h. The resulting reaction mixture was diluted with EtOAc (50 mL) and washed with 1 N HCl (50 mL) and brine. The organic layers were dried over anhydrous Na₂SO₄, filtered and concentrated to give the title compound 32-2 (495 mg, 86% yield) as a white solid, which was used at the next step without further purification.

Step 2: EDCI (218 mg, 1.14 mmol) was added to a solution of DMAP (7 mg, 0.06 mmol), compound 32-2 (190 mg, 0.57 mmol) and compound 31-1 (260 mg, 0.86 mmol) in DCM (10 mL) at 0° C. After stirring at room temperature for 2 h, the mixture was diluted with DCM (100 mL), washed with 1N HCl (50 mL) and brine (50 mL). The organic layer was dried over Na₂SO₄, filtered and concentrated to afford the title compound 32-3 (845 mg, 85% yield), which was used at the next step without further purification.

Step 3: Compound 32-3 (400 mg, 0.65 mmol) was dissolved in DCM (3 mL) and TFA (3 mL), the reaction mixture was stirred at room temperature for 2 h. The mixture was concentrated to afford the title compound 32-4 (240 mg, 91% yield) as a pale-yellow oil.

Step 4: A mixture of compound 31-4 (200 mg, 0.67 mmol), DMAP (8 mg, 0.07 mmol) and NHS (115 mg, 1.00 mmol) and EDCI (128 mg, 0.67 mmol) in anhydrous DMSO (3 mL) was stirred at room temperature for 1 h. To this mixture was added compound 32-4 (120 mg, 0.26 mmol) and Et₃N (0.483 mL, 3.35 mmol). The mixture was stirred at room temperature for 12 h and purified by prep-HPLC (eluted with 60% acetonitrile in water, HCl condition) to afford 32 (55 mg, 28% yield) as a white solid. ¹H NMR (400 MHz, DMSO-d₆) δ 8.60 (d, J=7.8 Hz, 1H), 8.01-7.90 (m, 1H), 7.79 (d, J=7.8 Hz, 2H), 7.67-7.58 (m, 2H), 7.31 (d, J=7.8 Hz, 2H), 7.01 (d, J=7.8 Hz, 2H), 6.34 (s, 1H), 4.45 (dd, J=9.3, 5.5 Hz, 1H), 4.03 (q, J=5.7 Hz, 2H), 3.28 (t, J=5.6 Hz, 2H), 3.00 (t, J=7.7 Hz, 2H), 2.88 (t, J=7.5 Hz, 2H), 2.46 (dd, J=19.7, 7.7 Hz, 4H), 2.22-2.11 (m, 1H), 2.07 (t, J=7.4 Hz, 2H), 2.04-1.94 (m, 1H), 1.77 (q, J=7.4 Hz, 2H). LCMS: m/z calculated for C₃₂H₃₅IN₆O₇: 742.57; found: 743.3. [M+H]⁺.

### Example 33

### (S)-2-(4-(2-(2-amino-4-oxo-4,7-dihydro-3H-pyrrolo[2,3-d]pyrimidin-5-yl)ethyl) benzamido)-5-(4-((S)-2-(4-isobutylphenyl)propanoyl)piperazin-1-yl)-5-oxopentanoic acid (33)

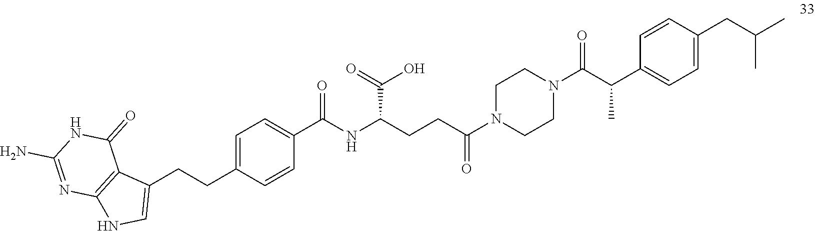 [SMILES: CC(C)Cc1ccc([C@H](C)C(=O)N2CCN(C(=O)CC[C@H](NC(=O)c3ccc(CCc4c[nH]c5nc(N)[nH]c(=O)c45)cc3)C(=O)O)CC2)cc1]

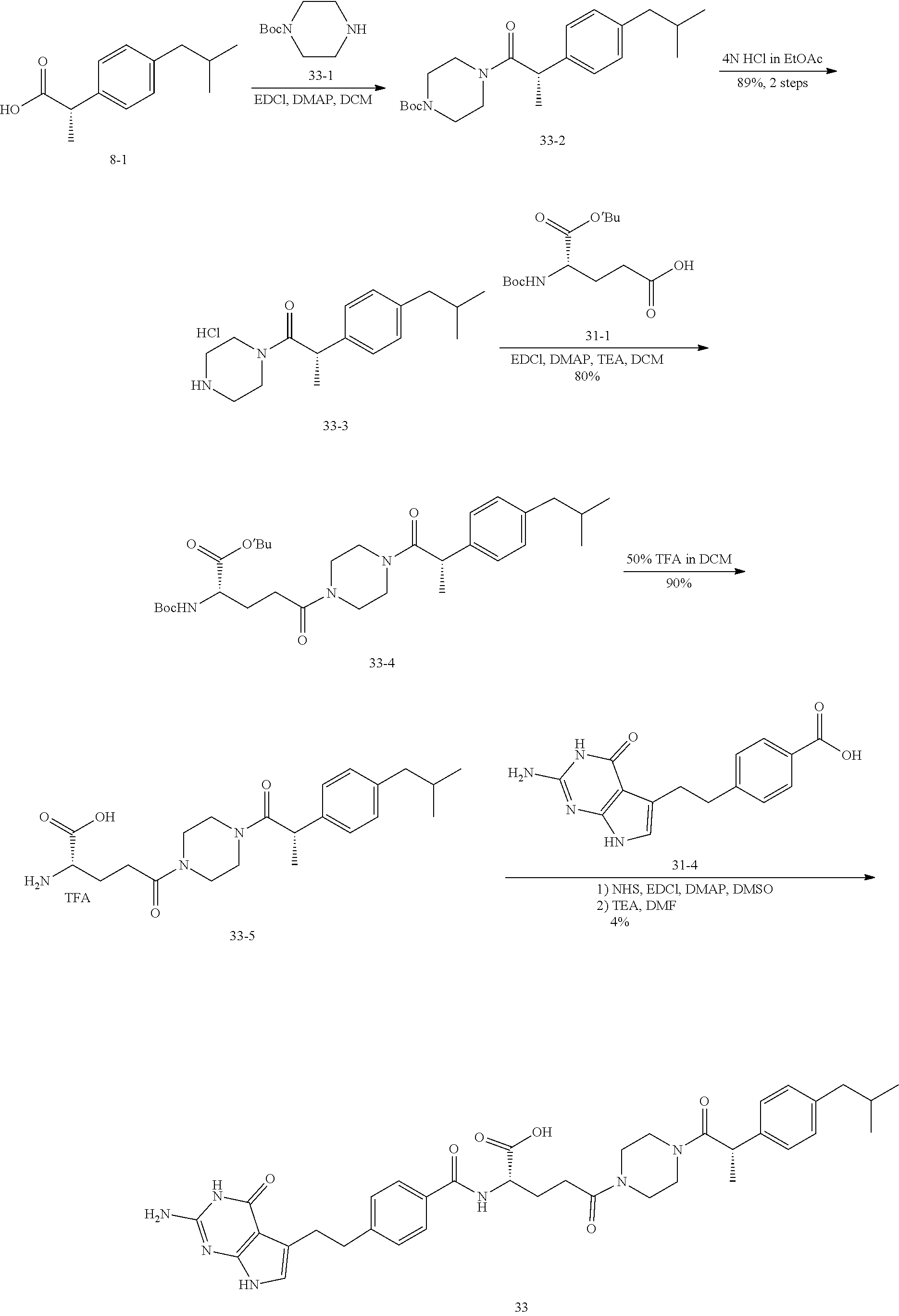

Step 1: EDCI (3.7 g, 19.37 mmol) was added to a mixture of acid 8-1 (2.00 g, 9.69 mmol), amine 33-1 (2.70 g, 14.49 mmol) and DMAP (118 mg, 0.967 mmol) in DCM (20 mL), the mixture was stirred at room temperature for 4 h. The reaction mixture was diluted with DCM (50 mL), washed with 1 N HCl (50 mL×2) and brine (50 mL×2). The organic layer was dried over anhydrous Na₂SO₄, filtered and concentrated to give the title compound 33-2 (3.62 g, crude) as a white solid, which was used at the next step without further purification. LCMS: m/z calculated for C₂₂H₃₄N₂O₃: 374.53; found: 397.47. [M+Na]⁺.

Step 2: To a solution of compound 33-2 (3.62 g, crude) in EtOAc (30 mL) was added 4 N HCl in EtOAc (30 mL). The mixture was stirred at room temperature for 1 h. The mixture was diluted with EtOAc (30 mL) and filtered. The precipitation was washed with EtOAc and dried to afford the title compound 33-3 (2.7 g, yield 89% over 2 steps) as a white solid. LCMS: m/z calculated for C₁₇H₂₆N₂O: 274.41; found: 275.90. [M+H]⁺.

Step 3: EDCI (3.12 g, 16.33 mmol) was added to a solution of DMAP (200 mg, 1.64 mmol), TEA (1.75 g, 17.32 mmol), compound 33-3 (2.70 g, 8.68 mmol) and compound 31-1 (2.48 g, 8.17 mmol) in DCM (40 mL) at 0° C. After stirring at room temperature for 2 h, the mixture was diluted with DCM (100 mL), washed with 1N HCl (50 mL) and brine (50 mL). The organic layer was dried over Na₂SO₄, filtered and concentrated. The crude product was purified by silica gel column (eluted with 50% EtOAc in petroleum ether) to afford the title compound 33-4 (3.66 g, 80% yield) as a white solid. LCMS: m/z calculated for C₃₁H₄₉N₃O₆: 559.75; found: 582.98. [M+Na]⁺.

Step 4: Compound 33-4 (2.88 g, 5.00 mmol) was dissolved in DCM (10 mL) and TFA (10 mL), the reaction was stirred at room temperature for 2 h. The mixture was concentrated to afford the title compound 33-5 (2.6 g, 90% yield) as a pale-yellow oil. LCMS: m/z calculated for C₂₂H₃₃N₃O₄: 403.52; found: 404.95. [M+H]⁺.

Step 5: A mixture of compound 31-4 (300 mg, 1.00 mmol), DMAP (12 mg, 0.098 mmol) and NHS (174 mg, 1.51 mmol) and EDCI (383 mg, 2.00 mmol) in anhydrous DMSO (3 mL) was stirred at room temperature for 2 h. After the activated NHS ester was formed completely, compound 33-5 (560 mg, 1.08 mmol) and Et₃N (508 mg, 5.02 mmol) was added. The mixture was stirred at 30° C. for 12 h and purified by prep-HPLC (eluted with 60% acetonitrile in water, TFA condition) to afford 33 (43 mg, 4% yield) as a white solid. ¹H NMR (400 MHz, DMSO-d₆ and D₂O) δ 7.75 (dd, J=8.0, 3.9 Hz, 2H), 7.28 (d, J=8.1 Hz, 2H), 7.13 (d, J=6.6 Hz, 2H), 7.08 (d, J=7.6 Hz, 2H), 6.37 (s, 1H), 4.34 (s, 1H), 4.05 (q, J=6.7 Hz, 1H), 3.58-3.48 (m, 4H), 3.48-3.34 (m, 4H), 2.97 (dd, J=9.3, 6.2 Hz, 2H), 2.86 (dd, J=9.6, 5.9 Hz, 2H), 2.38 (d, J=6.5 Hz, 4H), 1.99 (dt, J=40.0, 6.8 Hz, 2H), 1.84-1.71 (m, 1H), 1.26 (d, J=6.6 Hz, 3H), 0.81 (t, J=6.1 Hz, 6H). LCMS: m/z calculated for C₃₇H₄₅N₇O₆: 683.81; found: 684.65. [M+H]⁺.

### Example 34

### N2-(4-(2-(2-amino-4-oxo-4,7-dihydro-3H-pyrrolo[2,3-d]pyrimidin-5-yl)ethyl) benzoyl)-N₅-(2-((S)-2-(4-isobutylphenyl)propanamido)ethyl)-L-glutamine (34)

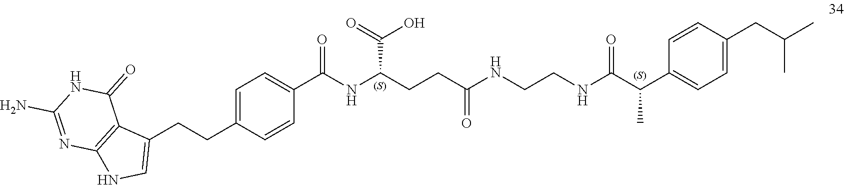

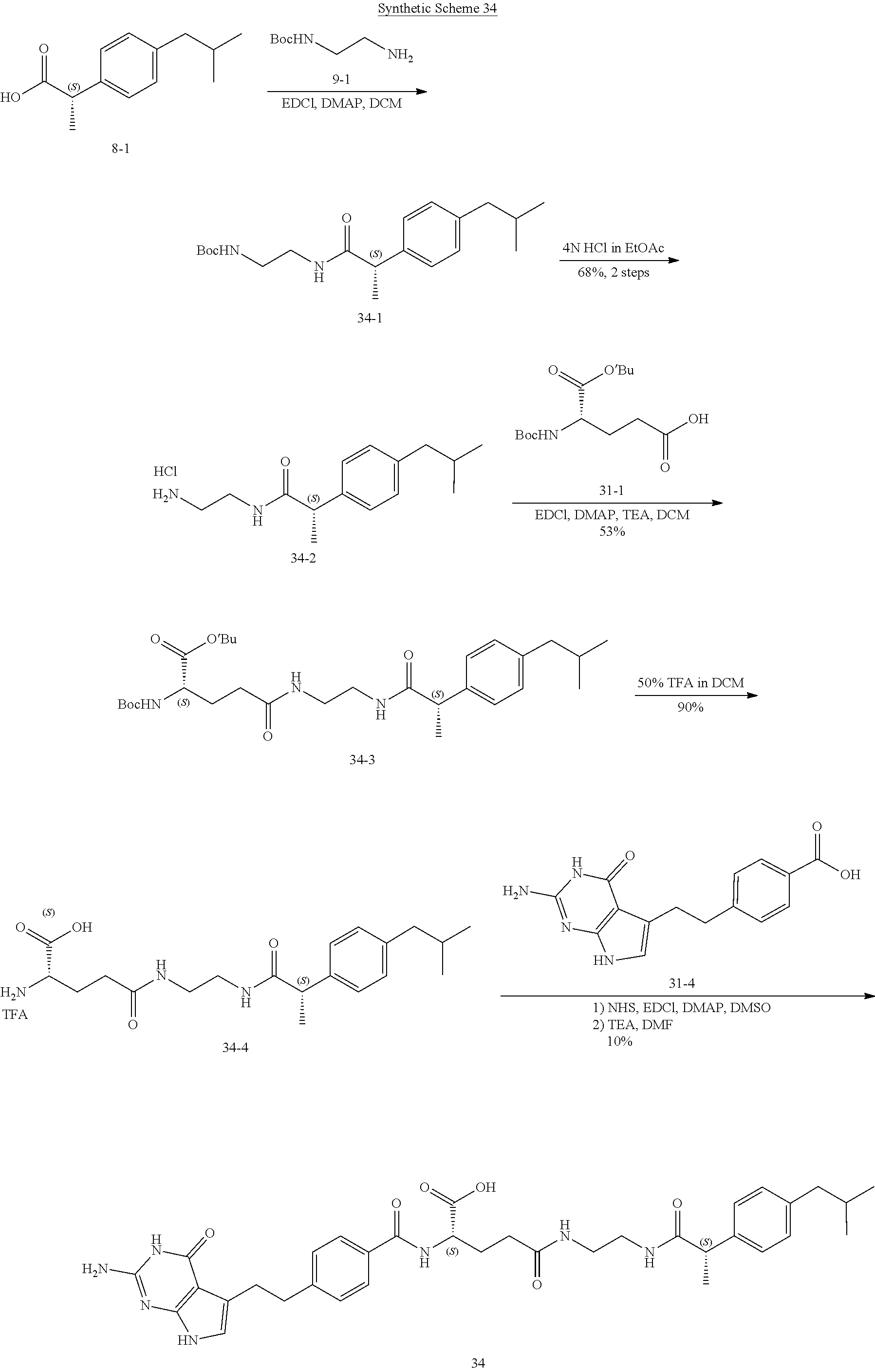

Step 1: EDCI (3.71 g, 19.42 mmol) was added to a mixture of acid 8-1 (2.00 g, 9.69 mmol), amine 9-1 (2.33 g, 14.54 mmol) and DMAP (238 mg, 1.95 mmol) in DCM (20 mL), the mixture was stirred at room temperature for 4 h. The reaction mixture was diluted with DCM (50 mL), washed with 1 N HCl (50 mL×2) and brine (50 mL×2). The organic layer was dried over anhydrous Na₂SO₄, filtered and concentrated to give the title compound 34-1 (3.2 g, crude) as a white solid, which was used at the next step without further purification. LCMS: m/z calculated for C₂₀H₃₂N₂O₃: 348.49; found: 397.47. [M+Na]⁺.

Step 2: To a solution of compound 34-1 (3.2 g, crude) in EtOAc (30 mL) was added 4 N HCl in EtOAc (30 mL), the mixture was stirred at room temperature for 1 h to form a white precipitation completely. The mixture was diluted with EtOAc (30 mL) and filtered. The precipitation was washed with EtOAc and dried to afford the title compound 34-2 (1.9 g, yield 68% over 2 steps) as a white solid. LCMS: m/z calculated for C₁₅H₂₄N₂O: 248.37; found: 249.90. [M+H]⁺.

Step 3: EDCI (1.28 g, 6.70 mmol) was added to a solution of DMAP (82 mg, 0.672 mmol), TEA (680 mg, 6.73 mmol), compound 34-2 (1.0 g, 3.51 mmol) and compound 31-1 (1.01 g, 3.32 mmol) in DCM (20 mL) at 0° C. The reaction was stirred at room temperature for 2 h, the mixture was diluted with DCM (50 mL), washed with 1N HCl (50 mL) and brine (50 mL). The organic layer was dried over Na₂SO₄, filtered and concentrated. The crude product was purified by silica gel column (eluted with 50% EtOAc in petroleum ether) to afford the title compound 34-3 (953 mg, 53% yield) as a white solid. LCMS: m/z calculated for C₂₉H₄₇N₃O₆: 533.71; found: 556.98. [M+Na]⁺.

Step 4: Compound 34-3 (700 mg, 1.31 mmol) was dissolved in DCM (7 mL) and TFA (7 mL), the reaction was stirred at room temperature for 2 h. The mixture was concentrated to afford the title compound 34-4 (700 mg, 90% yield) as a pale-yellow oil. LCMS: m/z calculated for C₂₀H₃₁N₃O₄: 377.49; found: 378.88. [M+H]⁺.

Step 5: A mixture of compound 31-4 (135 mg, 0.452 mmol), DMAP (5 mg, 0.0409 mmol) and NHS (78 mg, 0.678 mmol) and EDCI (172 mg, 0.900 mmol) in anhydrous DMSO (3 mL) was stirred at room temperature for 2 h. After the NHS activated ester was formed completely, compound 34-4 (310 mg, 0.630 mmol) and Et₃N (230 mg, 2.27 mmol) was added. The mixture was stirred at 30° C. for 12 h and purified by prep-HPLC (eluted with 60% acetonitrile in water, TFA condition) to afford 34 (26 mg, 6% yield) as a white solid. ¹H NMR (400 MHz, DMSO-d₆ and D₂O) δ 7.81 (dd, J=18.1, 7.9 Hz, 2H), 7.30 (dd, J=11.5, 7.9 Hz, 2H), 7.19-7.15 (m, 2H), 7.06-7.02 (m, 2H), 6.36 (s, 1H), 4.33 (dd, J=10.2, 4.6 Hz, 1H), 3.14-3.00 (m, 4H), 2.99-2.94 (m, 2H), 2.86 (dd, J=9.3, 5.8 Hz, 2H), 2.37 (d, J=7.1 Hz, 2H), 2.18 (t, J=7.5 Hz, 2H), 2.14-1.86 (m, 2H), 1.82-1.70 (m, 1H), 1.28 (d, J=6.9 Hz, 3H), 0.84-0.79 (m, 6H). LCMS: m/z calculated for C₃₅H₄₃N₇O₆: 657.77; found: 658.86. [M+H]⁺.

### Example 35

### trans-[Pt(DACH)(ox)(OH)(IPBA)](35, IPBA=4-(4-iodophenyl)butanoic acid) (35)

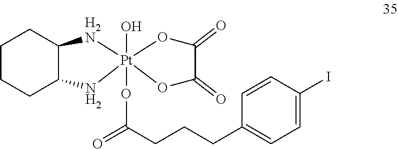

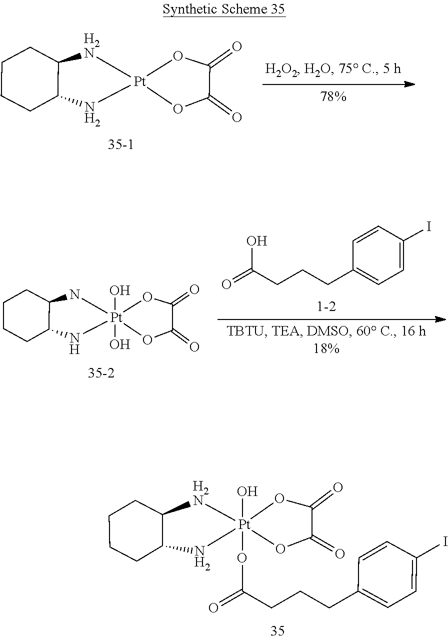

Step 1: Hydrogen peroxide (30%, 15 mL) was added dropwise to a suspension of oxaliplatin 35-1 (200 mg, 0.50 mmol) in water (5 ml). After addition, the reaction mixture was heated to 75° C. and stirred for 5 hours. A clear solution was formed and cooled to room temperature. The resulting solution was concentrated. The residue was washed with EtOH and MTBE to give the title compound 35-2 (170 mg, yield 78%) as a yellow solid.

Step 2: To a solution of acid 1-2 (114 mg, 0.39 mmol) and TBTU (127 mg, 0.39 mmol) in anhydrous DMSO (5 mL) was added TEA (55 uL, 0.39 mmol). The mixture was intensively stirred at room temperature for 15 min. Compound 35-2 (170 mg, 0.39 mmol) was added and the reaction mixture was stirred at 60° C. for 16 h. The resulting reaction mixture was filtered to remove un-reacted solid. The clear solution was purified by reverse phase flash chromatography (C18 column, eluted with 50% acetonitrile in water, neutral condition). The desired components were lyophilized overnight to afford 35 (50.8 mg, yield 18%) as a white solid. ¹H NMR (400 MHz, DMSO-d6) δ 7.59 (d, J=7.8 Hz, 2H), 6.97 (d, J=7.8 Hz, 2H), 2.44 (d, J=7.6 Hz, 2H), 2.15 (d, J=7.5 Hz, 2H), 2.06 (t, J=17.4 Hz, 2H), 1.68 (t, J=7.5 Hz, 2H), 1.56-0.99 (m, 8H).

### Example 36

### trans-[Pt(DACH)(ox)(IPBA)₂](36, IPBA=4-(4-iodophenyl)butanoic acid) (36)

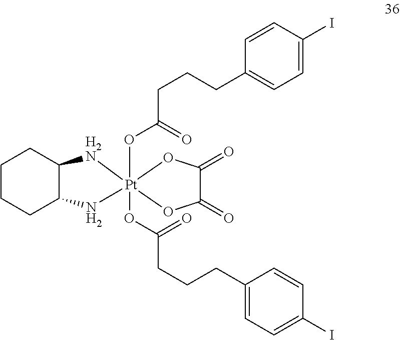

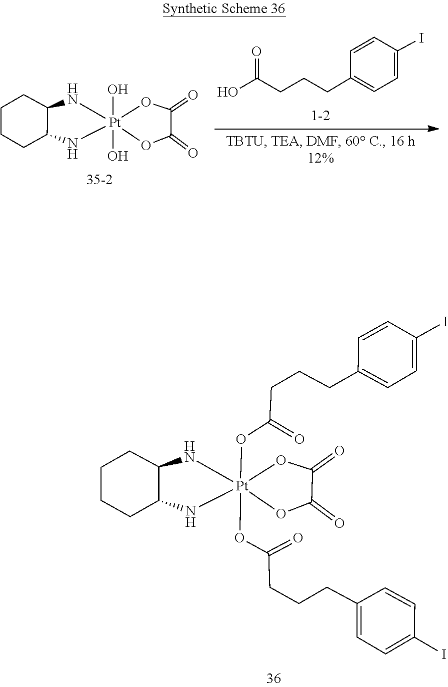

Step 1: A mixture of compound 1-2 (587 mg, 2.03 mmol), TBTU (650 mg, 2.02 mmol) and TEA (205 mg, 2.02 mmol) in DMF (5 mL) was stirred at room temperature under nitrogen for 15 min. To this mixture was added compound 35-2 (218 mg, 0.506 mmol) in one portion. The resulting reaction was stirred at 60° C. for 16 h, the mixture was filtered to remove un-reacted solid. The clear solution was directly purified by reverse phase flash chromatography (C18 column, acetonitrile and water, neutral condition) to afford 36 (60 mg, yield 12%) as a white solid. ¹H NMR (400 MHz, DMSO-d₆) δ 7.60 (dd, J=8.0, 3.8 Hz, 4H), 6.97 (dd, J=8.2, 3.8 Hz, 4H), 2.47 (s, 4H), 2.23 (q, J=6.6 Hz, 4H), 2.10 (d, J=12.9 Hz, 2H), 1.77-1.62 (m, 4H), 1.53-1.03 (m, 8H). LCMS: m/z calculated for C₂₈H₃₄I2N₂O₈Pt: 975.48; found: 976.57 [M+H]⁺.

### Example 37

### N-((S)-1-(((R)-1-(6-(2-(2-(2-((S)-2-(4-isobutylphenyl)propanamido)ethoxy)ethoxy)ethyl)-4,8-dioxo-1,3,6,2-dioxazaborocan-2-yl)-3-methylbutyl)amino)-1-oxo-3-phenylpropan-2-yl)pyra zine-2-carboxamide (37)

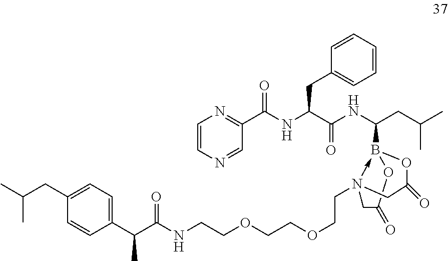 [SMILES: CC(C)Cc1ccc([C@H](C)C(=O)NCCOCCOCC[N+]23CC(=O)O[B-]2([C@H](CC(C)C)NC(=O)[C@H](Cc2ccccc2)NC(=O)c2cnccn2)OC(=O)C3)cc1]

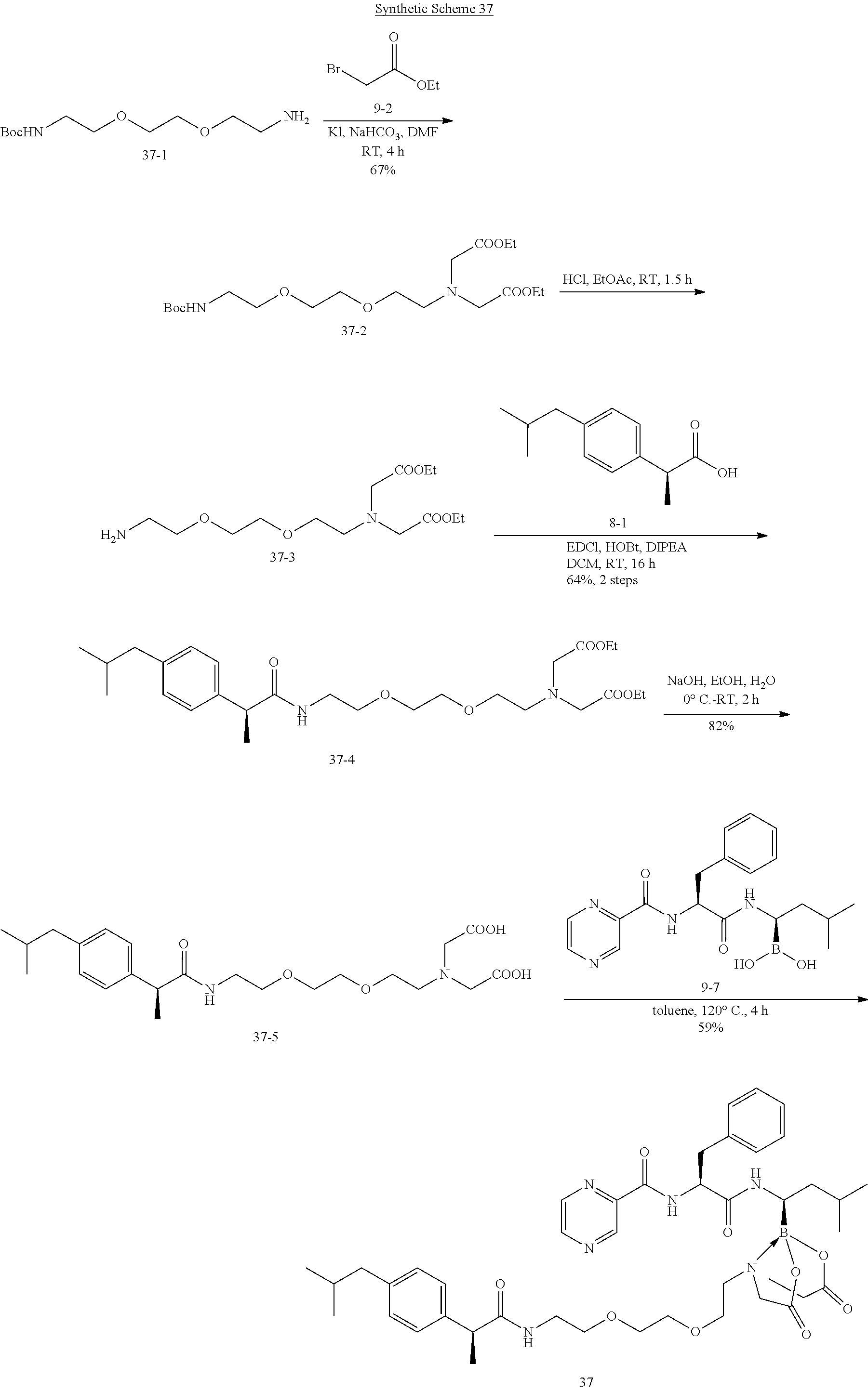

Step 1: To a solution of compound 37-1 (1.0 g, 4.02 mmol) in DMF (10 mL) was added NaHCO₃ (3.38 g, 40.23 mmol), KI (669 mg, 4.03 mmol) and compound 9-2 (3.36 g, 20.11 mmol). The mixture was stirred at room temperature for 4 h. After completion of the reaction, the mixture was partitioned between EtOAc (100 mL) and water (100 mL). The organic layer was washed with brine, dried over anhydrous Na₂SO₄ and concentrated. The residue was purified by silica gel column (eluted with 50% EtOAc in petroleum ether) to afford the title compound 37-2 (1.14 g, yield 67%). LCMS: m/z calculated for C₁₉H₃₆N₂O₈: 420.50; found: 443.85 [M+Na]⁺.

Step 2: A mixture of compound 37-2 (1.14 g, 2.71 mmol) in 4 N HCl (EtOAc solution, 10 mL) was stirred at room temperature for 1.5 h. After completion of the reaction, the mixture was concentrated to afford the title compound 37-3 (1.36 g, crude), which was used at the next step without further purification.

Step 3: A solution of compound 37-3 (1.36 g, crude), compound 8-1 (670 mg, 3.24 mmol), DIPEA (525 mg, 4.06 mmol), HOBt (360 mg, 2.66 mmol) and EDCI (520 mg, 2.71 mmol) in DCM (10 mL) was stirred at room temperature for 16 h. After completion of the reaction, the mixture was diluted with DCM (100 mL), washed with water (100 mL×2) and brine (100 mL). The organic layer was dried over anhydrous Na₂SO₄, filtered and concentrated. The crude product was purified by reverse phase flash chromatography (C18 column, eluted with 65% acetonitrile in water, HCl condition). The desired components were lyophilized to afford the title compound 37-4 (948 mg, TFA salt, yield 64% over 2 steps) as a white solid. LCMS: m/z calculated for C₂₇H₄₄N₂O₇: 508.66; found: 531.73 [M+Na]⁺.

Step 4: To a solution of compound 37-4 (945 mg, 1.73 mmol) in EtOH (9.5 mL) and H₂O (3 mL) was added NaOH (297 mg, 7.45 mmol) at 0° C. The reaction was stirred at room temperature for 2 h. The mixture was concentrated and adjusted to pH=2 with 1 N HCl at 0° C. The aqueous mixture was purified by reverse phase flash chromatography (C18 column, eluted with 45% acetonitrile in water, HCl condition). The desired components were lyophilized to afford the title compound 37-5 (694 mg, yield 82%) as a white solid. LCMS: m/z calculated for C₂₃H₃₆N₂O₇: 452.55; found: 453.52 [M+H]⁺.

Step 5: A mixture of compound 37-5 (100 mg, 0.204 mmol) and compound 9-7 (85 mg, 0.22 mmol) in toluene (3 mL) was stirred at 120° C. for 4 h. After completion of the reaction, the mixture was concentrated. The crude product was purified by pre-TLC (acetonitrile:dichloromethane=1:3) to afford 37 (97 mg, yield 59%) as a white solid. ¹H NMR (400 MHz, DMSO-d₆) δ 9.05 (s, 1H), 8.92 (d, J=7.8 Hz, 1H), 8.84 (s, 1H), 8.71 (s, 1H), 7.95 (s, 1H), 7.37 (d, J=9.3 Hz, 1H), 7.29-7.12 (m, 7H), 7.03 (d, J=6.9 Hz, 2H), 4.66 (d, J=6.2 Hz, 1H), 4.28 (d, J=17.6 Hz, 1H), 4.02 (dd, J=32.9, 17.2 Hz, 2H), 3.68 (t, J=18.8 Hz, 3H), 3.46 (s, 3H), 3.39-3.25 (m, 4H), 3.16 (s, 2H), 3.09-3.00 (m, 2H), 2.37 (s, 2H), 1.76 (s, 1H), 1.55 (s, 1H), 1.41 (t, J=12.4 Hz, 1H), 1.27 (d, J=6.3 Hz, 3H), 1.23-1.16 (m, 1H), 0.82 (d, J=6.5 Hz, 12H). LCMS: m/z calculated for C₄₂H₅₇BN₆O₉: 800.76; found: 801.76 [M+H]⁺.

### Example 38

### (S)-4,11-diethyl-4-hydroxy-3,14-dioxo-3,4,12,14-tetrahydro-1H-pyrano[3′,4′:6,7]indolizino[1,2-b]quinolin-9-yl(S)-2-(4-(2-(methyl-d3)propyl-3,3,3-d3)phenyl)propanoate (38)

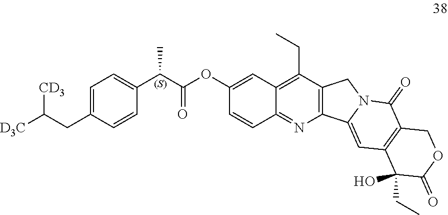

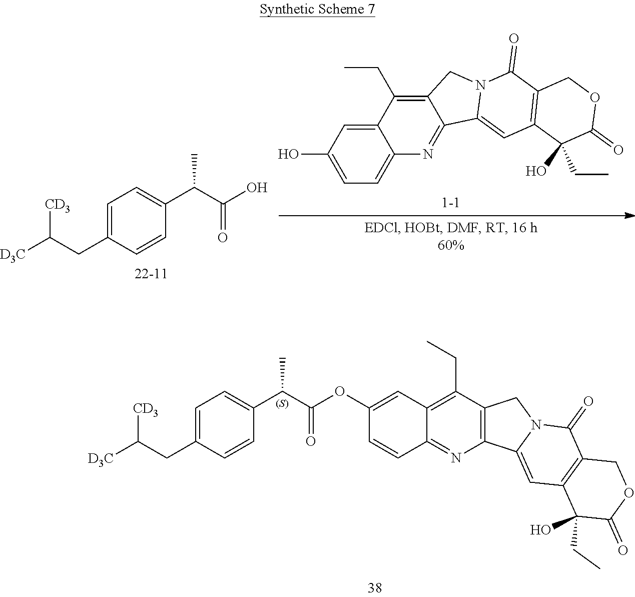

Step 1: A mixture of compound 22-11 (30.0 mg, 0.141 mmol), EDCI (80.0 mg, 0.419 mmol) and HOBt (20.0 mg, 0.148 mmol) in anhydrous DMF (2 mL) was stirred at room temperature for 5 min. Compound 1-1 (85 mg, 0.216 mmol) was added and the mixture was stirred at room temperature for 16 h. The reaction mixture was partition between EtOAc (30 mL) and water (20 mL). The organic layer was washed with brine, dried over anhydrous Na₂SO₄, filtered and concentrated. The crude product was purified by pre-TLC (5% MeOH in DCM) to afford the title compound 38 (50 mg, yield 60%) as an off-white solid. ¹H NMR (500 MHz, DMSO-d₆) δ 8.18 (d, J=9.1 Hz, 1H), 7.90 (d, J=2.5 Hz, 1H), 7.51 (dd, J=9.1, 2.5 Hz, 1H), 7.37 (d, J=7.9 Hz, 2H), 7.32 (s, 1H), 7.20 (d, J=7.8 Hz, 2H), 5.43 (s, 2H), 5.32 (s, 2H), 4.15 (q, J=7.1 Hz, 1H), 3.16 (q, J=7.6 Hz, 2H), 2.45 (d, J=7.2 Hz, 2H), 1.85 (ddt, J=28.9, 14.6, 7.2 Hz, 3H), 1.57 (d, J=7.1 Hz, 3H), 1.27 (t, J=7.6 Hz, 3H), 0.88 (t, J=7.3 Hz, 3H). LCMS: m/z calculated for C₃₅H₃₀D₆N₂O₆: 586.72; found: 588.13 [M+H]⁺.

### Example 39. Cellular IC50 in Three Tumor Cell Lines

The cellular inhibition was determined in three assays: U87MG, A549 and MC38. Cells were recovered and cultured in appropriate medium supplemented with 10% fetal bovine serum and 100 U/mL penicillin G sodium, and maintained in cell incubators (37° C., 5% CO2). Before testing, cells in culture dishes were rinsed with Phosphate Buffered Solution, detached with Trypsin. Dilute and adjust the cell number with the culture medium, add the cell suspension to the 96 well cell plate. Cells were maintained in incubators overnight. In T0 control plate, cells were added with 100 μL CellTiter-Glo reagent, balance at room temperature for 10 minutes, and read the chemiluminescence value with envision.

In the testing plate, compounds were dissolved with corresponding solvent and diluted gradiently. Diluted compound solution was added to a 96 well plate with cells. After incubation of 72 h, cells were added with 100 μL CellTiter-Glo reagent, balanced at room temperature for 10 minutes, and read the chemiluminescence value with envision. IC50 was calculated using the GraphPad Prism software package (Prism 6 for Windows, Version 6.0, GraphPad Software Inc., San Diego, CA). The IC₅₀ for the three cellular assays is shown in Table 1.

TABLE 1
IC50

| | U87MG | A549 | MC38 |
|---|---|---|---|
| Compound | (IC50, nM) | (IC50, nM) | (IC50, nM) |
| 8 | 13.37693 | 207.81632 | 22.74152 |
| 16 | 7.266483 | 10.68657 | 15.94872 |
| 9-7 (Valcade ) | 6.890381 | 9.36019 | 16.29071 |
| 1-1 (SN-38) | 13.58112 | 99.519661 | 21.24506 |
| 5-1 | NA | NA | NA |
| 8-1 | #Intersect | #Intersect | >10000 |
| 16-2 | #Intersect | #Intersect | #Intersect |

A: <1 μM;
B: 1-10 μM.
C: >10 μM

### Example 40. Chemical Stability in PBS (pH 7.4)

The chemical stability assay was performed according to the following procedures.
1. Test compounds spiking solution: 1 mM test compounds spiking solution A: Add 10 μL of 10 mM test compounds stock solution to 90 μL DMSO.
2. Add 396 μL of buffer into the tubes designated for different time points. Pre-warm the samples at 37° C. for 10 min.
3. Add 4 μL of spiking solution A into the wells designated as 0 min (or 15, 45, 90, 120 min) containing 396 μL of buffer, then start timing count down.
4. At each time point, add 1200 μL ACN containing IS into the tubes.
5. Samples are centrifuged at 10,000 rpm for 5 min, then 100 μL of supernatant are ready for LC-MS/MS analysis.

TABLE 2
Stability

| | Stability (PBS, pH |
|---|---|
| Compound # | 7.4) T½ (min) |
| 16 | ∞ |
| 8 | ∞ |
| 4 | 215 |
| 30 | 806 |
| 31 | ∞ |
| 1 | 1182 |
| 2 | 1387 |
| 3 | 51 |
| 28 | 504 |

### Example 41. Pharmacokinetic

Pharmacokinetic parameters were determined in male CD-1 mice (Shanghai Jihui Laboratory Animal Care Co., Ltd., 6-8 weeks old). Animals were maintained under a 12 hr light/dark cycle. Animals had free access to food and water during the study.

For intravenous, compounds were formulated as a solution (in 5% DMSO+10% Solutol HS15+85% (20% HP-β-CD in water)) in 5 mL/kg dosing volume and administered via tail vein. For subcutaneous, compounds were formulated as a solution (in 5% DMSO+10% Solutol HS15+85% (20% HP-β-CD in water)) in 10 mL/kg dosing volume or (in 0.5% MC+1% Pluronic F68 in water) in 50 μL/mouse dosing volume and administered via subcutaneous puncture.

Semi-serial blood samples (about 110 μL) were taken from animal at 0.083, 0.25, 0.5, 1, 2, 4, 8 and 24 hr for IV group and 0.5, 2, 4, 8, 24, 48 and 96 hr for SC group. Samples were held on ice for no longer than 15 minutes before centrifugation (2000 g, 5 min, 4° C.) within 15 minutes post sampling. Plasma was snap frozen in dry ice and then transferred into −70° C. freezer for long term storage until LC-MS/MS analysis.

Tissue collection for SC group at 0.5, 2, 4, 8, 24, 48 and 96 hr. After animals were anesthetized and exsanguinated, tissue samples (including muscle and skin) were collected and weighted, and then snap frozen in liquid nitrogen and further stored at −70° C. for long term storage until LC-MS/MS analysis. Tissue samples were homogenized under freezing conditions.

PK parameters were generated from LC-MS/MS data using Phoenix WinNonlin 8.2 software.

TABLE 3
Pharmacokinetic Data

| Dosing | Dose | Compound | | AUClast | AUClast | Tissue/Plasma |
|---|---|---|---|---|---|---|
| compound | (mg/kg) | measured | T½ (h) | (hr*ng/mL) | (hr*ng/mL) | ratio |
| 1 | 3 | 1 active drug | 10.3 | 24402 | 142 | 171.8450704 |
| | | 1-1 | | | | |
| 8 | 5 | 8 | 44.1 | 11834033 | NA | #VALUE! |
| 8 | 5 | 8 active drug | >96 | 255830 | 201 | 1272.78607 |
| | | 1-1 | | | | |
| 28 | 10 | 28 | 11.3 | 37582 | NA | #VALUE! |
| 28 | 10 | active drug | >48 | 4094 | NA | #VALUE! |
| | | 28-1 | | | | |
| 30 | 10 | active drug | 25.4 | 144212 | 5274 | 27.34395146 |
| | | 30-1 | | | | |
| 31 | 10 | active drug | 34.2 | 979 | 203 | 4.822660099 |
| | | 31-4 | | | | |
| 33 | 10 | active drug | NA | 9139 | 504 | 18.13293651 |
| | | 33-1 | | | | |
| 16 | 1 | 16 | 93.2 | 60283 | 165 | 365.3515152 |
| 16 | 1 | active drug | >96 | 5776 | 13.5 | 427.8518519 |
| | | 9-7 | | | | |
| 10 | 1 | active drug | 22.4 | 5919 | 10.2 | 580.2941176 |
| | | 9-7 | | | | |
| 13 | 1 | active drug | 22 | 11243 | 284 | 39.58802817 |
| | | 9-7 | | | | |

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A compound having the structural formula of (I) or (II): 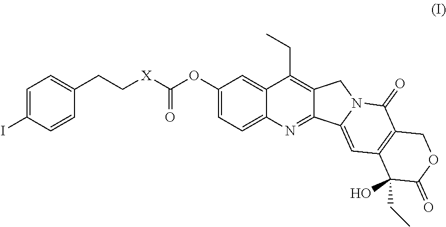
wherein
X is CHR or NR, and
R is H or alkyl, 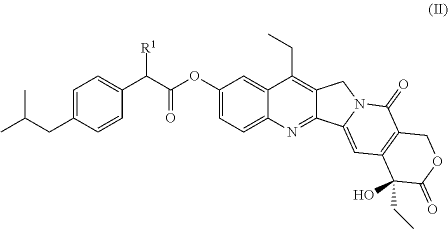
wherein R¹ is methyl,
or a pharmaceutically acceptable form or an isotope derivative thereof.

2. The compound of claim 1, selected from:

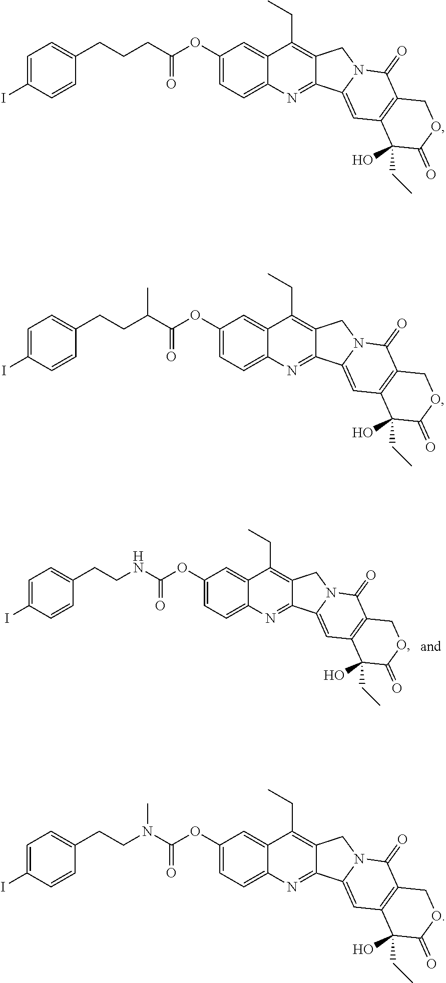 [SMILES: CCc1c2c(nc3ccc(OC(=O)CCCc4ccc(I)cc4)cc13)-c1cc3c(c(=O)n1C2)COC(=O)[C@]3(O)CC ; CCc1c2c(nc3ccc(OC(=O)C(C)CCc4ccc(I)cc4)cc13)-c1cc3c(c(=O)n1C2)COC(=O)[C@]3(O)CC ; CCc1c2c(nc3ccc(OC(=O)NCCc4ccc(I)cc4)cc13)-c1cc3c(c(=O)n1C2)COC(=O)[C@]3(O)CC ; CCc1c2c(nc3ccc(OC(=O)N(C)CCc4ccc(I)cc4)cc13)-c1cc3c(c(=O)n1C2)COC(=O)[C@]3(O)CC]

3. (canceled)

4. The compound of claim 1, selected from:

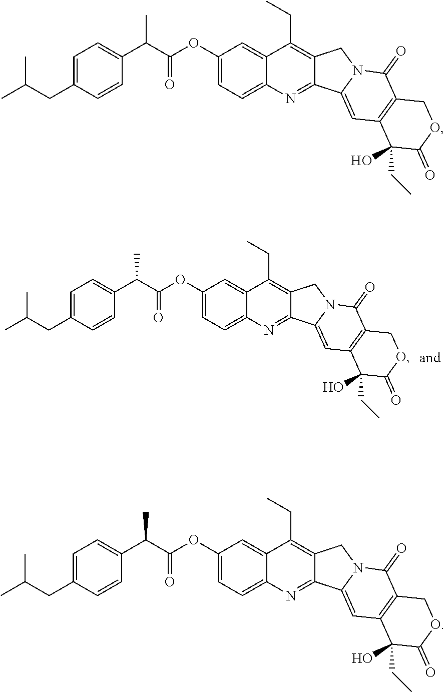 [SMILES: CCc1c2c(nc3ccc(OC(=O)C(C)c4ccc(CC(C)C)cc4)cc13)-c1cc3c(c(=O)n1C2)COC(=O)[C@]3(O)CC ; CCc1c2c(nc3ccc(OC(=O)[C@@H](C)c4ccc(CC(C)C)cc4)cc13)-c1cc3c(c(=O)n1C2)COC(=O)[C@]3(O)CC ; CCc1c2c(nc3ccc(OC(=O)[C@H](C)c4ccc(CC(C)C)cc4)cc13)-c1cc3c(c(=O)n1C2)COC(=O)[C@]3(O)CC]

5. (canceled)

6. A compound having the structural formula (III) or (IV): 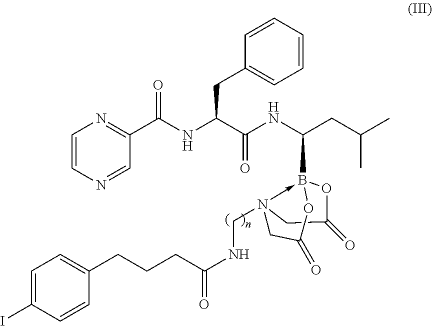
wherein n is an integer selected from 2-8, 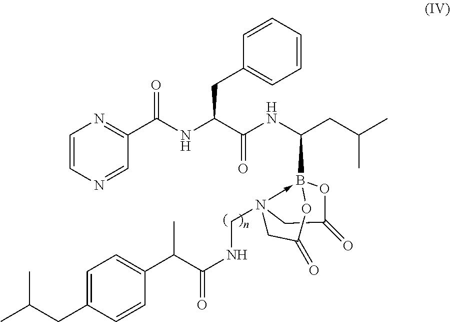
wherein n is an integer selected from 2-10,
or a pharmaceutically acceptable form or an isotope derivative thereof.

7. The compound of claim 6, selected from:

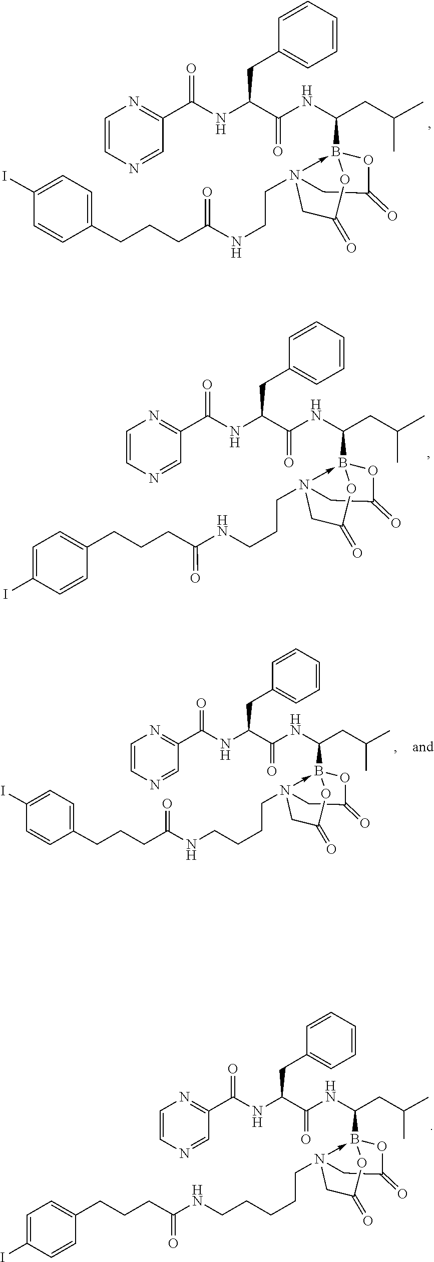 [SMILES: CC(C)C[C@H](NC(=O)[C@H](Cc1ccccc1)NC(=O)c1cnccn1)[B-]12OC(=O)C[N+]1(CCNC(=O)CCCc1ccc(I)cc1)CC(=O)O2 ; CC(C)C[C@H](NC(=O)[C@H](Cc1ccccc1)NC(=O)c1cnccn1)[B-]12OC(=O)C[N+]1(CCCNC(=O)CCCc1ccc(I)cc1)CC(=O)O2 ; CC(C)C[C@H](NC(=O)[C@H](Cc1ccccc1)NC(=O)c1cnccn1)[B-]12OC(=O)C[N+]1(CCCCNC(=O)CCCc1ccc(I)cc1)CC(=O)O2 ; CC(C)C[C@H](NC(=O)[C@H](Cc1ccccc1)NC(=O)c1cnccn1)[B-]12OC(=O)C[N+]1(CCCCCNC(=O)CCCc1ccc(I)cc1)CC(=O)O2]

8. (canceled)

9. The compound of claim 7, selected from:

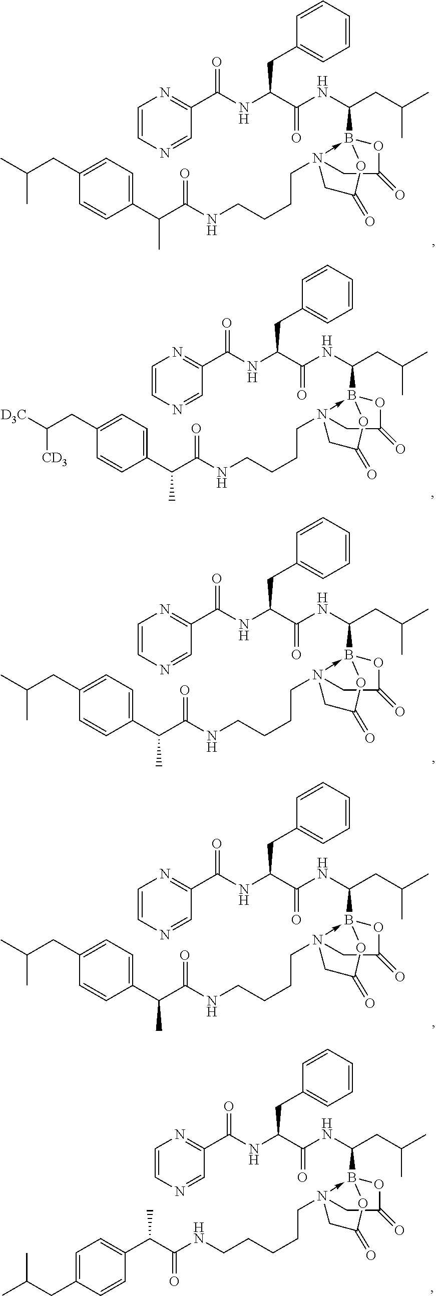

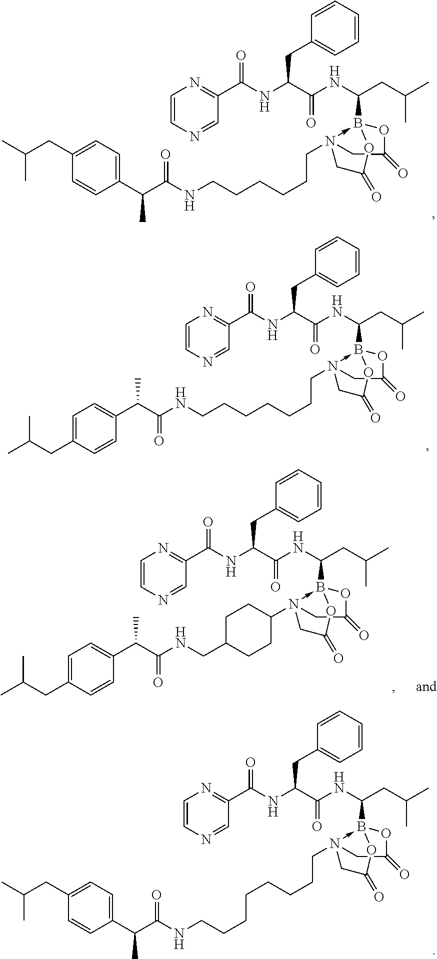

10. A compound having the structural formula (V): 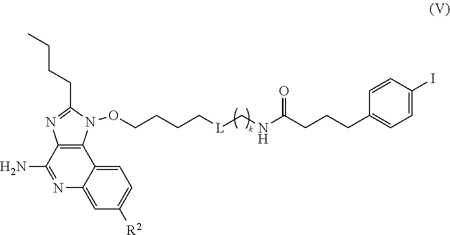
wherein
R² is H or P(═O)R₂, and R is alkyl,
L is a single bond or a group selected from: 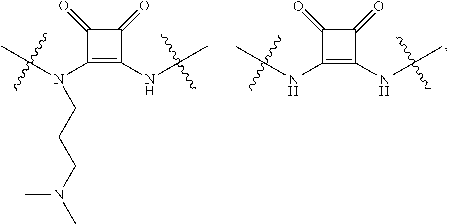 [SMILES: *Nc1c(N(*)CCCN(C)C)c(=O)c1=O ; *Nc1c(N*)c(=O)c1=O]
and
k is an integer selected from 0-4, 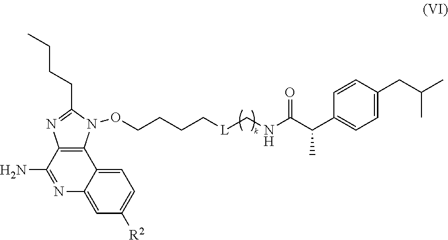
wherein
R² is H or P(═O)R₂, and R is alkyl,
L is a single bond or a group selected from: 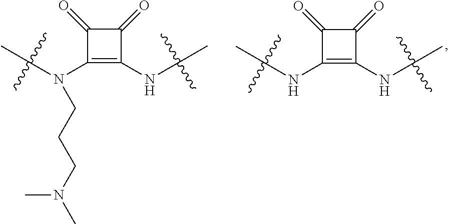 [SMILES: *Nc1c(N(*)CCCN(C)C)c(=O)c1=O ; *Nc1c(N*)c(=O)c1=O]
and
k is an integer selected from 0-4, 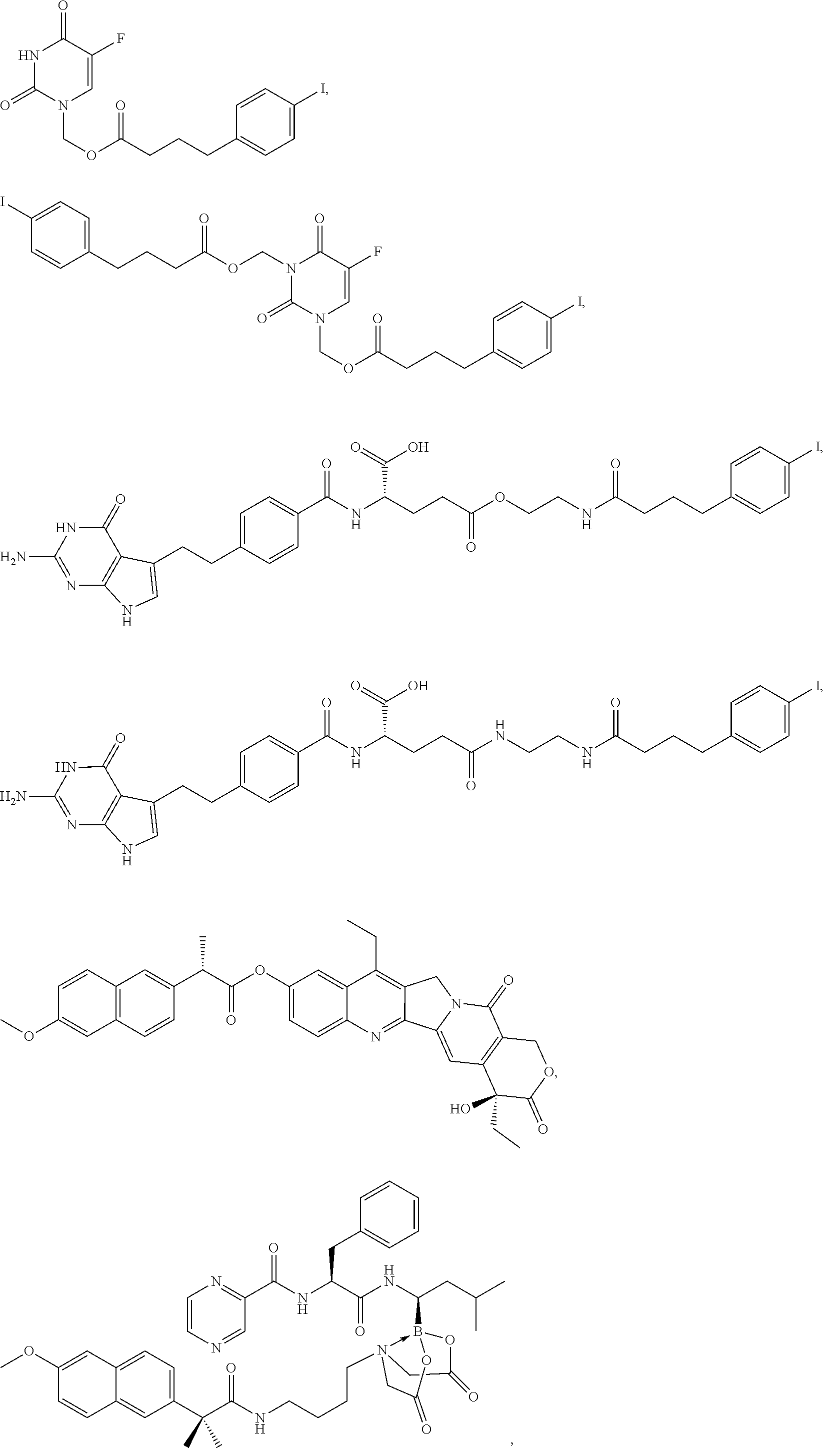 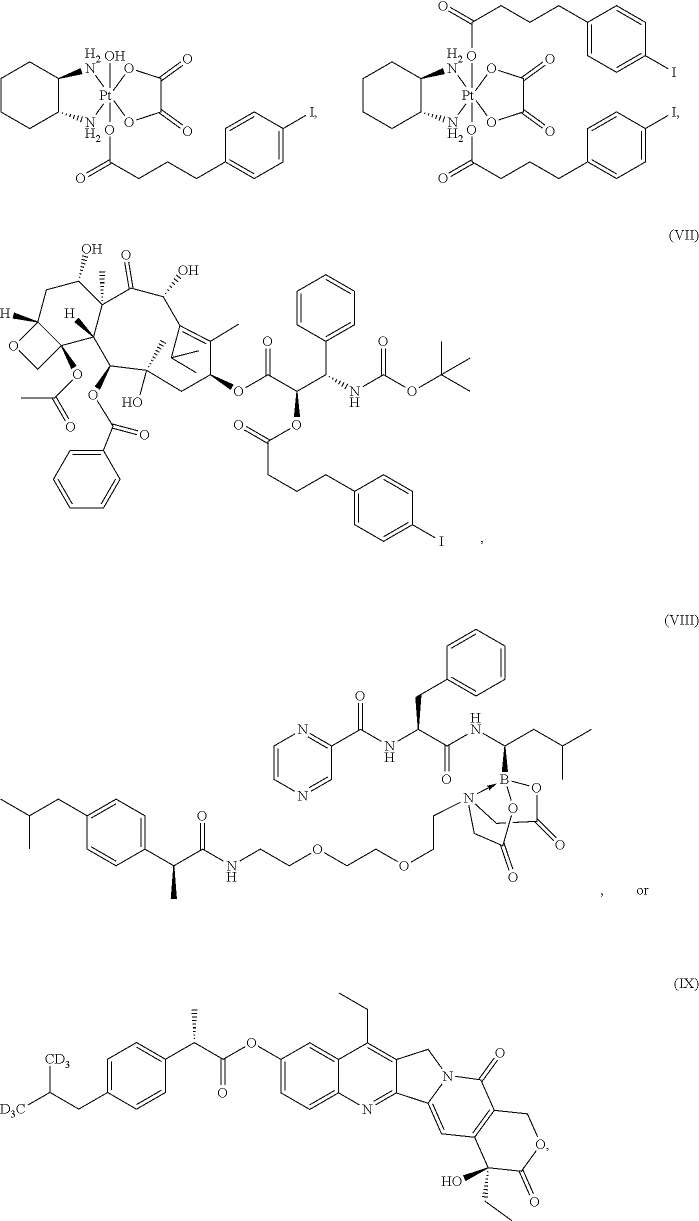
or a pharmaceutically acceptable form or an isotope derivative thereof.

11. The compound of claim 10, selected from:

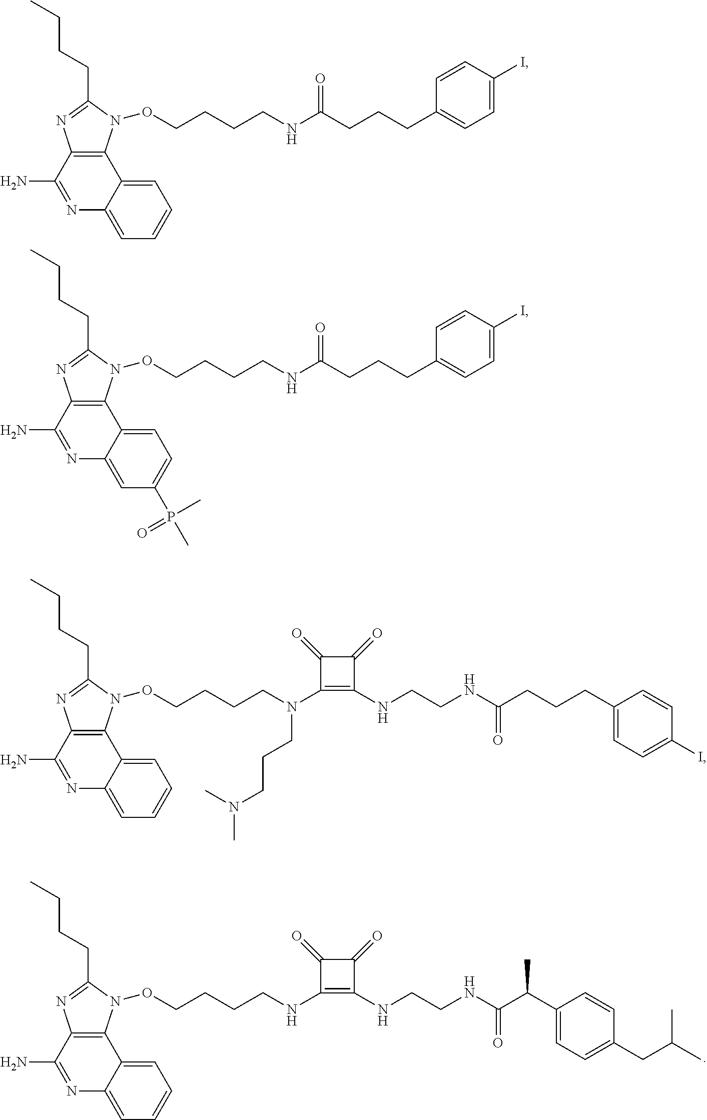 [SMILES: CCCCc1nc2c(N)nc3ccccc3c2n1OCCCCNC(=O)CCCc1ccc(I)cc1 ; CCCCc1nc2c(N)nc3cc(P(C)(C)=O)ccc3c2n1OCCCCNC(=O)CCCc1ccc(I)cc1 ; CCCCc1nc2c(N)nc3ccccc3c2n1OCCCCN(CCCN(C)C)c1c(NCCNC(=O)CCCc2ccc(I)cc2)c(=O)c1=O ; CCCCc1nc2c(N)nc3ccccc3c2n1OCCCCNc1c(NCCNC(=O)[C@@H](C)c2ccc(CC(C)C)cc2)c(=O)c1=O]

12. (canceled)

13. The compound of claim 10, having the structure:

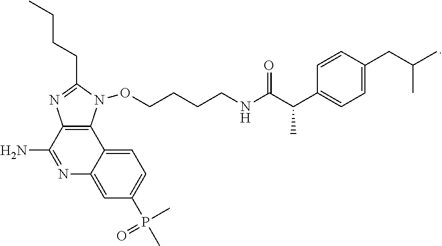 [SMILES: CCCCc1nc2c(N)nc3cc(P(C)(C)=O)ccc3c2n1OCCCCNC(=O)[C@@H](C)c1ccc(CC(C)C)cc1]

14-19. (canceled)

20. A pharmaceutical composition comprising a compound according to claim 1 and a pharmaceutically acceptable excipient, carrier, or diluent.

21. The pharmaceutical composition of claim 20, effective to treat or reduce cancer, or a related disease or condition.

22. A unit dosage form comprising a pharmaceutical composition according to claim 20.

23. A method for treating or reducing a disease or condition, comprising administering to a subject in need thereof a pharmaceutical composition comprising a compound according to claim 1 and a pharmaceutically acceptable excipient, carrier, or diluent.

24. The method of claim 23, wherein the disease or condition is cancer, or a related disease or condition thereof.

25-27. (canceled)
